(19) United States
(12) Patent Application Publication
(10) Pub. No.: US 2017/0266211 A1
(43) Pub. Date: Sep. 21, 2017

(54) USE OF A HETEROCYCLIC BCL-2 INHIBITOR FOR REMOVING SENESCENT CELLS AND TREATING SENESCENCE-ASSOCIATED CONDITIONS

(71) Applicants: Unity Biotechnology, Inc., Brisbane, CA (US); Buck Institute for Research on Aging, Novato, CA (US)

(72) Inventors: Nathaniel David, San Francisco, CA (US); Remi-Martin Laberge, S. San Francisco, CA (US)

(21) Appl. No.: 15/611,589

(22) Filed: Jun. 1, 2017

Related U.S. Application Data
(63) Continuation of application No. PCT/US16/16894, filed on Feb. 5, 2016.
(60) Provisional application No. 62/113,227, filed on Feb. 6, 2015.

(51) Int. Cl. A61K 31/675

(52) U.S. Cl. CPC A61K 31/675

(57) ABSTRACT

Disclosed herein are compounds that are effective for treatment of various disease states. Dosing includes both single administration and regimens of cycling dosages.

### CROSS-REFERENCE TO RELATED APPLICATIONS

This application is a continuation of PCT Application No. PCT/US2016/16894, filed Feb. 5, 2016, which claims the benefit of U.S. provisional patent application No. 62/113,227, filed Feb. 6, 2015, incorporated herein by reference herein in its entirety.

### BACKGROUND

Senescent cells accumulate in tissues and organs of individuals as they age and are found at sites of age-related pathologies. Senescent cells are believed important to inhibiting proliferation of dysfunctional or damaged cells and particularly to constraining development of malignancy (see, e.g., Campisi, Curr. Opin. Genet. Dev. 21: 107-12 (2011); Campisi, Trends Cell Biol. 11:S27-31 (2001); Prieur et al, Curr. Opin. Cell Biol. 20: 150-55 (2008)); nevertheless, the presence of senescent cells in an individual may contribute to aging and aging-related dysfunction (see, e.g., Campisi, Cell 120:513-22 (2005)). Given that senescent cells have been causally implicated in certain aspects of age-related decline in health and may contribute to certain diseases, and are also induced as a result of necessary life-preserving chemotherapeutic and radiation treatments, the presence of senescent cells may have deleterious effects to millions of patients worldwide. However, identifying and developing treatments of such diseases and conditions by selective elimination of senescent cells has been an arduous undertaking. The present disclosure addresses these needs and offers related advantages.

### SUMMARY

The disclosure provides methods of treating senescence-associated conditions comprising administered a compound or salt described herein. In certain embodiments, the disclosure provides method for treating a senescence-associated condition comprising administering to a subject in need thereof a compound represented by Formula (III), (IV) or (V):

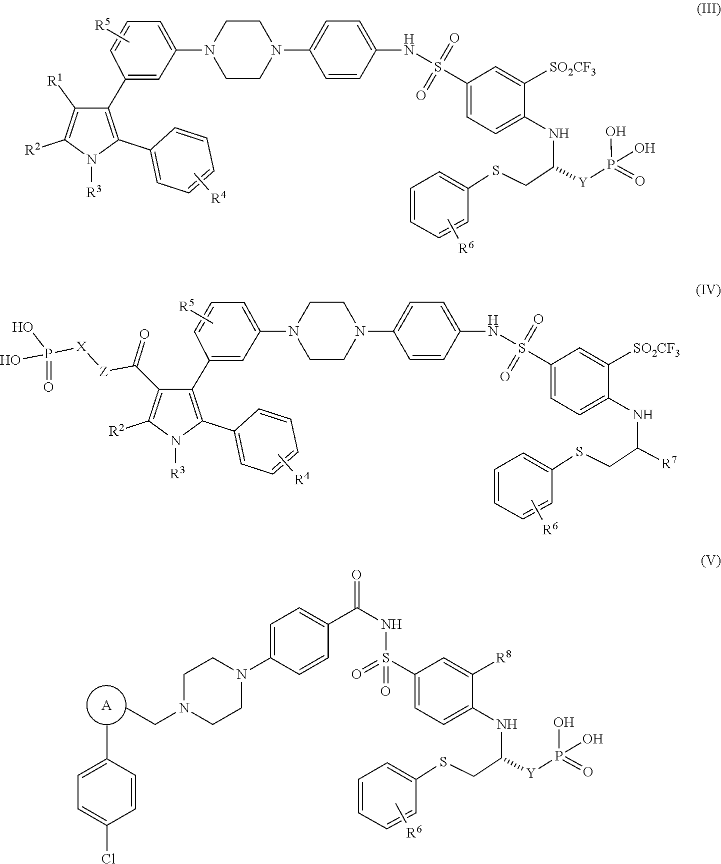

or a salt thereof, wherein the senescence-associated condition is not cancer and the compound or salt is administered at an amount less than a dose effective for cancer treatment, wherein:

the A ring is

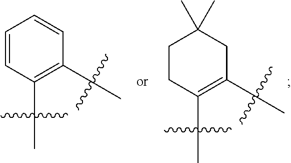

X, substituted or unsubstituted, is selected from the group consisting of alkylene, alkenylene, cycloalkylene, cycloalkenylene, and heterocycloalkylene;

Y is selected from the group consisting of (CH₂)ₙ—N(R^{a}) and

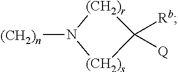

Q is selected from the group consisting of O, O(CH₂)₁₋₃, NR^{c}, NR^{c}(C₁₋₃alkylene), OC(═O)(C₁₋₃alkylene), C(═O)O, C(═O)O(C₁₋₃alkylene), NHC(═O)(C₁₋₃alkylene), C(═O)NH, and C(═O)NH(C₁₋₃alkylene);

Z is O or NR^{c};

R₁ and R₂, independently, are selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, COR′, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;

R₃ is selected from a group consisting of H, alkyl, cycloalkyl, alkenyl, cycloalkenyl, alkynyl, aryl, heteroaryl, heterocycloalkyl, OR′, NR′R″, OCOR′, CO₂R′, COR′, CONR′R″, CONR′SO₂R″, C₁₋₃alkyleneCH(OH)CH₂OH, SO₂R′, and SO₂NR′R″;

R′, R″, and R′″, independently, are H, alkyl, cycloalkyl, alkenyl, cycloalkenyl, alkynyl, aryl, heteroaryl, C₁₋₃alkyleneheterocycloalkyl, or heterocycloalkyl;

R′ and R″, or R″ and R′″, can be taken together with the atom to which they are bound to form a 3 to 7 membered ring;

R₄ is hydrogen, halo, C₁₋₃alkyl, CF₃, or CN;

R₅ is hydrogen, halo, C₁₋₃alkyl, substituted C₁₋₃alkyl, hydroxyalkyl, alkoxy, or substituted alkoxy;

R₆ is selected from the group consisting of H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;

R₇, substituted or unsubstituted, is selected from the group consisting of hydrogen, alkyl, alkenyl, (CH₂)₀₋₃cycloalkyl, (CH₂)₀₋₃cycloalkenyl, (CH₂)₀₋₃heterocycloalkyl, (CH₂)₀₋₃aryl, and (CH₂)₀₋₃heteroaryl;

R₈ is selected from the group consisting of hydrogen, halo, NO₂, CN, SO₂CF₃, and CF₃;

Rₐ is selected from the group consisting of hydrogen, alkyl, heteroalkyl, alkenyl, hydroxyalkyl, alkoxy, substituted alkoxy, cycloalkyl, cycloalkenyl, and heterocycloalkyl;

R_{b} is hydrogen or alkyl;

R_{c} is selected from the group consisting of hydrogen, alkyl, substituted alkyl, hydroxyalkyl, alkoxy, and substituted alkoxy; and

n, r, and s, independently, are 1, 2, 3, 4, 5, or 6.

In certain embodiments, the disclosure provides a method for treating a senescence-associated condition comprising administering to a subject in need thereof a compound represented by Formula (III), (IV) or (V), as described herein, or a salt thereof, wherein the senescence-associated condition is not cancer and the compound or salt is administered in at least two treatment cycles, wherein each treatment cycle independently comprises a treatment course of from 1 day to 3 months followed by a non-treatment interval of at least 2 weeks.

In certain embodiments, the disclosure provides a method for treating a senescence-associated condition comprising administering to a subject in need thereof a compound represented by Formula (III), (IV) or (V), as described herein, or a salt thereof, wherein the senescence-associated condition is not cancer and the compound or salt is administered in a single dose.

A method for treating a senescence-associated condition comprising administering to a subject in need thereof a compound represented by Formula (I):

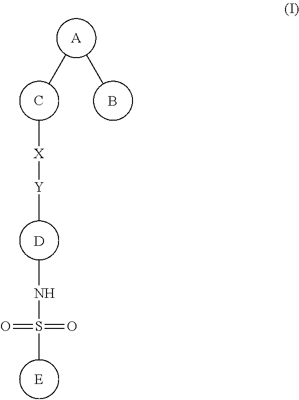

or a salt thereof, wherein the senescence-associated disease or disorder is not cancer and the compound or salt is administered at an amount less than a dose effective for cancer treatment, wherein:

A is absent, optionally substituted phenyl, or an optionally substituted five or six-membered aromatic ring in which 1 to 4 carbon atoms individually are replaced by nitrogen, oxygen, or sulfur;

B, C, D, and E individually are optionally substituted phenyl or an optionally substituted five or six-membered aromatic ring in which 1 to 4 carbon atoms individually are replaced by nitrogen, oxygen, or sulfur;

X and Y, independently, are absent, O, S, CO, SO₂, SO, PO₃H, NR′, BR′, PR′, POR′, alkylene, cycloalkylene, alkenylene, cycloalkenylene, alkynylene, or arylene; or X and Y can be taken together to form a 5-7 membered ring, or X and Y can be Z—(CH₂)₁₋₃—Z′, wherein Z and Z′, independently, are O, S, NR, CO, SO, SO₂, PO₃H, PR′, or POR′; and

R′ is H, alkyl, cycloalkyl, alkenyl, cycloalkenyl, alkynyl, aryl, heteroaryl, or heterocycloalkyl.

In certain embodiments, the compound of Formula (I) is represented by Formula (II):

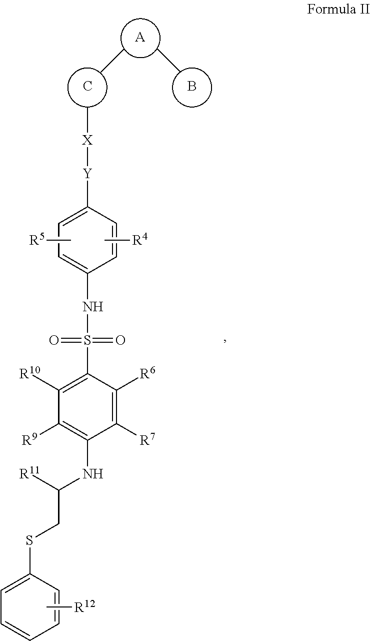

wherein, R₁₁ and R₁₂, independently, are selected from H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R″, CONSO₂R′R″, NR′COR″, NR′CONR″R′″, NR′C═SNR″R′″, NR′SO₂R″, SO₂R′, and SO₂NR′R″.

In certain embodiments, the disclosure provides a method for treating a senescence-associated condition comprising administering to a subject in need thereof a compound represented by Formula (I) or (II), as described herein, or a salt thereof, wherein the senescence-associated condition is not cancer and the compound or salt is administered in at least two treatment cycles, wherein each treatment cycle independently comprises a treatment course of from 1 day to 3 months followed by a non-treatment interval of at least 2 weeks.

In certain embodiments, the disclosure provides a method for treating a senescence-associated condition comprising administering to a subject in need thereof a compound represented by Formula (I), or (II) as described herein, or a salt thereof, wherein the senescence-associated condition is not cancer and the compound or salt is administered in a single dose.

### DETAILED DESCRIPTION

Aging is a risk factor for most chronic diseases, disabilities, and declining health. Senescent cells, which are cells in replicative arrest, accumulate as an individual ages and may contribute, partially or significantly to cell and tissue, deterioration that underlies aging and age related diseases. Cells may also become senescent after exposure to an environmental, chemical, or biological insult or as a result of a disease. Provided herein are methods and agents for selective killing of senescent cells that are associated with numerous pathologies and diseases, including age-related pathologies and diseases. As disclosed herein, senescent cell associated diseases and disorders may be treated or prevented (i.e., likelihood of occurrence or development is reduced) by administering at least one seno lytic agent. The senescent cell-associated disease or disorder treated or prevented by the agents and methods described herein include a cardiovascular disease or disorder, inflammatory or autoimmune disease or disorder, a pulmonary disease or disorder, a neurological disease or disorder, a dermatological disease or disorder, a chemotherapeutic side effect, a radiotherapy side effect, or metastasis, or a metabolic disease, all of which are described in greater detail herein. In certain specific embodiments, the senescent cell-associated diseases or disorders treated or prevented by the senolytic agents and methods described herein include, by way of example, idiopathic pulmonary fibrosis (WE), chronic obstructive pulmonary disease (COPD), osteoarthritis, and cardiovascular diseases and disorders associated with arteriosclerosis, such as atherosclerosis. In certain embodiments, the senescence associated disease or disorder is not a cancer. As described in greater detail herein, senolytic agents include, for example, MDM2 inhibitors (e.g., nutlin 3a, RG-7112); inhibitors of one or more BCL-2 anti-apoptotic protein family members, which inhibitors inhibit a function of at least the anti-apoptotic protein, BCL-xL (e.g., ABT-263, ABT-737, WEHI-539, A-1155463); and Akt specific inhibitors (e.g., MK-2206).

Senolytic agents described herein are sufficient to kill significant numbers of senescent cells. Even though cells continue to become senescent in a treated subject, establishment of senescence, such as shown by the presence of a senescence-associated secretory phenotype (SASP), occurs over several days (see, e.g., Laberge et al, Aging Cell 11:569-78 (2012); Coppe et al, PLoS Biol. 6: 2853-68 (2008); Coppe et al. PLoS One 5:39188 (2010); Rodier et al, Nat. Cell Biol. 11:973-9′19; Freund et al, EMBO J. 30: 15364548 (2011)). Use of the senolytic agents described herein, therefore, offers the advantage that these agents can be administered less frequently, intermittently, and/or at a lower dose than many therapeutic agents commonly used for treating these diseases and disorders. The methods described herein describe use of such agents as senolytic agents that may be administered less frequently, intermittently, and/or at a lower dose than when the agents are used for treating cancer other diseases.

### Indications.

The present disclosure provides methods for treating a disease or disorder comprising administering a compound described herein to a subject in need thereof. The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In some embodiments, the disease or disorder is a cardiovascular disease or disorder, inflammatory disease or disorder, autoimmune disease or disorder, pulmonary disease or disorder, eye disease or disorder, metabolic disease or disorder, neurological disease or disorder, neurodegenerative disease or disorder, age-related disease or disorder, skin condition, dermatological disease or disorder, transplant related disease or disorder, a premature aging disease or disorder, or a sleep disorder.

In some embodiments, an age-related disease or disorder can be associated with a gradual loss of function, and degeneration that can occur at the molecular, cellular, tissue, or organismal level. Age-related degeneration can give rise to pathologies including, for example, sarcopenia, atherosclerosis and heart failure, osteoporosis, pulmonary insufficiency, renal failure, neurodegeneration, macular degeneration, Alzheimer's disease, and Parkinson's disease. Different mammalian species can vary in their susceptibilities to specific age-related pathologies.

In some embodiments, the disease or disorder is a cognitive disease, for example, mild cognitive impairment (MCI), Alzheimer's disease, Huntington's disease, dementia; a cardiovascular disease, for example, atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, or cardiac fibrosis; a metabolic disease or disorder, for example, obesity, diabetes, or metabolic syndrome; a motor function disease or disorder, for example, Parkinson's disease, motor neuron dysfunction (MND), or Huntington's disease; a cerebrovascular disease, emphysema, osteoarthritis, benign prostatic hypertrophy; a pulmonary disease or disorder, for example, idiopathic pulmonary fibrosis, chronic obstructive pulmonary disease (COPD), emphysema, obstructive bronchiolitis, or asthma; an inflammatory/autoimmune disease or disorder, for example, osteoarthritis, eczema, psoriasis, osteoporosis, mucositis, transplantation related diseases and disorders; an ophthalmic disease or disorder, for example, age-related macular degeneration, cataracts, glaucoma, vision loss, presbyopia; diabetic ulcer, metastasis; a chemotherapeutic side effect, a radiotherapy side effect; aging-related disease or disorder, for example, kyphosis, renal dysfunction, frailty, hair loss, hearing loss, muscle fatigue, skin conditions, sarcopenia, and herniated intervertebral disc, and a disease/disorder resulting from irradiation, chemotherapy, smoking tobacco, eating a high fat/high sugar diet, and environmental factors; wound healing; skin nevi; a fibrotic disease and disorder, for example, cystic fibrosis, renal fibrosis, liver fibrosis, pulmonary fibrosis, oral submucous fibrosis, cardiac fibrosis, and pancreatic fibrosis.

### Cardiovascular Diseases and Disorders

Methods for treating a disease or disorder comprising administering a compound described herein to a subject in need thereof wherein the disease or disorder is a cardiovascular disease or disorder are disclosed. Cardiovascular diseases or disorders, include, but are not limited to angina, arrhythmia, atherosclerosis, cardiomyopathy, congestive heart failure, coronary artery disease (CAD), carotid artery disease, endocarditis, heart attack, coronary thrombosis, myocardial infarction NIL high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease, peripheral artery disease (PAD), cardiac stress resistance, and stroke.

In some embodiments, the disease or disorder is associated with or caused by arteriosclerosis. The cardiovascular disease can be any one or more of atherosclerosis, for example, coronary artery disease (CAD) and carotid artery disease; angina, congestive heart failure, and peripheral vascular disease, for example, peripheral artery disease (PAD). The methods for treating a cardiovascular disease that is associated with or caused by arteriosclerosis can reduce the likelihood of occurrence of high blood pressure/hypertension, angina, stroke, and heart attack, coronary thrombosis, and myocardial infarction (MI). In certain embodiments, methods are provided for stabilizing atherosclerotic plaque(s) in a blood vessel, for example, an artery, of a subject, thereby reducing the likelihood of occurrence or delaying the occurrence of a thrombotic event, such as stroke or MI. In certain embodiments, these methods comprise administration of a compound described herein, to reduce the lipid content of an atherosclerotic plaque in a blood vessel, for example, an artery, of the subject, or increase the fibrous cap thickness by, for example, causing an increase, enhancing or promoting thickening of the fibrous cap.

Atherosclerosis is characterized by patchy intimal plaques, atheromas, that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and branches thereof, and major arteries of the extremities.

In one embodiment, methods are provided for inhibiting, reducing, or causing a decrease in, the formation of atherosclerotic plaques by administering a compound described herein to a subject in need thereof. In some embodiments, methods are provided for reducing, decreasing, or diminishing the amount, or level, of plaque. Reduction in the amount of plaque in a blood vessel, for example, an artery, can be determined, for example, by a decrease in surface area of the plaque, or by a decrease in the extent, degree, or percent of occlusion of a blood vessel, for example, an artery, which can be determined by angiography or other visualizing methods.

Also provided herein are methods for increasing, improving, promoting, or enhancing the stability of atherosclerotic plaques that are present in one or more blood vessels, for example, one or more arteries of a subject, which methods comprise administering to the subject any one of the compounds described herein.

Atherosclerosis can be a hardening or furring of the arteries and is caused by the formation of multiple atheromatous plaques within the arteries. Atherosclerosis (also called arteriosclerotic vascular disease or ASVD) is a form of arteriosclerosis in which an artery wall thickens. Symptoms develop when growth or rupture of the plaque reduces or obstructs blood flow; and the symptoms can vary depending on which artery is affected. Atherosclerotic plaques can be stable or unstable. Stable plaques regress, remain static, or grow slowly, sometimes over several decades, until they can cause stenosis or occlusion. Unstable plaques are vulnerable to spontaneous erosion, fissure, or rupture, causing acute thrombosis, occlusion, and infarction long before they cause hemodynamically significant stenosis. Clinical events can result from unstable plaques, which do not appear severe on angiography; thus, plaque stabilization can be a way to reduce morbidity and mortality. Plaque rupture or erosion can lead to major cardiovascular events such as acute coronary syndrome and stroke. Disrupted plaques can have a greater content of lipid, macrophages, and have a thinner fibrous cap than intact plaques.

Atherosclerosis is a syndrome affecting arterial blood vessels due in significant part to a chronic inflammatory response of white blood cells in the walls of arteries. This is promoted by low-density lipoproteins (LDL), plasma proteins that carry cholesterol and triglycerides, in the absence of adequate removal of fats and cholesterol from macrophages by functional high-density lipoproteins (HDL). The earliest visible lesion of atherosclerosis is the “fatty streak,” which is an accumulation of lipid-laden foam cells in the intimal layer of the artery. The hallmark of atherosclerosis is atherosclerotic plaque, which is an evolution of the fatty streak and has three major components: lipids, for example, cholesterol and triglycerides; inflammatory cells and smooth muscle cells; and a connective tissue matrix that can contain thrombi in various stages of organization and calcium deposits. Within the outer-most and oldest plaque, calcium and other crystallized components, for example, microcalcification from dead cells can be found. Microcalcification and properties related thereto are also thought to contribute to plaque instability by increasing plaque stress. Fatty streaks reduce the elasticity of the artery walls, but cannot affect blood flow for years because the artery muscular wall accommodates by enlarging at the locations of plaque. Lipid-rich atheromas are at increased risk for plaque rupture and thrombosis. The lipid core can exhibit the highest thrombogenic activity. Within major arteries in advanced disease, the wall stiffening can also eventually increase pulse pressure.

A vulnerable plaque that can lead to a thrombotic event, for example, stroke or MI, and is sometimes described as a large, soft lipid pool covered by a thin fibrous cap. An advanced characteristic feature of advance atherosclerotic plaque is irregular thickening of the arterial intima by inflammatory cells, extracellular lipid (atheroma) and fibrous tissue (sclerosis). Fibrous cap formation can occur from the migration and proliferation of vascular smooth muscle cells and from matrix deposition. A thin fibrous cap contributes instability of the plaque and to increased risk for rupture.

Both proinflammatory macrophages (M1) and anti-inflammatory macrophages (M2) can be found in arteriosclerotic plaque. The contribution of both types to plaque instability is a subject of active investigation, with results suggesting that an increased level of the M1 type versus the M2 type correlates with increased instability of plaque.

Diagnosis of atherosclerosis and other cardiovascular disease can be based on symptoms, for example, angina, chest pressure, numbness or weakness in arms or legs, difficulty speaking or slurred speech, drooping muscles in face, leg pain, high blood pressure, kidney failure and/or erectile dysfunction, medical history, and/or physical examination of a patient. Diagnosis can be confirmed by angiography, ultrasonography, or other imaging tests. Subjects at risk of developing cardiovascular disease include those having any one or more of predisposing factors, such as a family history of cardiovascular disease and those having other risk factors, for example, predisposing factors including high blood pressure, dyslipidemia, high cholesterol, diabetes, obesity and cigarette smoking, sedentary lifestyle, and hypertension. In a certain embodiment, the disease or disorder is atherosclerosis.

One or any combination of diagnostic methods, including physical examination, assessment and monitoring of clinical symptoms, and performance of analytical tests and methods described herein, for example, angiography, electrocardiography, stress test, or non-stress test, can be used for monitoring the health status of the subject. The effects of the treatment of a compound described herein or pharmaceutical composition comprising same can be analyzed by, for example, comparing symptoms of patients suffering from or at risk of cardiovascular disease that have received the treatment with those of patients without such a treatment or with placebo treatment.

### Inflammatory and Autoimmune Diseases and Disorders

In some embodiments, methods for treating a disease or disorder comprising administering a compound described herein to a subject in need thereof wherein the disease or disorder is an inflammatory or autoimmune disease or disorder are disclosed. Inflammatory or autoimmune diseases or disorders, include, but are not limited to, osteoarthritis, osteoporosis, psoriasis, oral mucositis, rheumatoid arthritis, inflammatory bowel disease, eczema, kyphosis, herniated intervertebral disc, pulmonary diseases, COPD, and idiopathic pulmonary fibrosis.

Osteoarthritis degenerative joint disease is characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. Symptoms of osteoarthritis include sore or stiff joints, particularly the hips, knees, and lower back, after inactivity or overuse; stiffness after resting that goes away after movement; and pain that is worse after activity or toward the end of the day. Osteoarthritis can also affect the neck, small finger joints, the base of the thumb, ankle, and big toe.

Chronic inflammation can be an age-related factor that contributes to osteoarthritis. In combination with aging, joint overuse and obesity can promote osteoarthritis.

In some embodiments, a compound described herein, reduces or inhibits loss or erosion of proteoglycan layers in a joint, reduces inflammation in the affected joint, and promotes, stimulates, enhances, or induces production of collagen, for example, type 2 collagen. In some embodiments, the administration of a compound described herein to a subject in need thereof causes a reduction in the amount, or level, of inflammatory cytokines, such as IL-6, produced in a joint and inflammation is reduced. Methods are provided herein for treating osteoarthritis and/or inducing collagen, for example, Type 2 collagen, production in the joint of a subject in need thereof by administering at least one compound described herein, which can be combined with at least one pharmaceutically acceptable excipient to form a pharmaceutical composition, to the subject. A compound also can be used for decreasing, inhibiting, or reducing production of metalloproteinase 13 (MMP-13), which degrades collagen in a joint, and for restoring proteoglycan layer or inhibiting loss and/or degradation of the proteoglycan layer. Treatment with a compound described herein thereby also reduces the likelihood of, inhibits, or decreases erosion, or slows erosion of the bone. As described in detail herein, in certain embodiments, a compound described herein is administered directly to an osteoarthritic joint, for example, intra-articularly, topically, transdermally, intradermally, or subcutaneously. Treatment with a compound described herein can also restore, improve, or inhibit deterioration of strength of a joint. In addition, the methods comprising administering a compound described herein can reduce joint pain and are therefore useful for pain management of osteoarthritic joints.

The compounds described herein can be used for treatment of osteoarthritis in a subject and monitoring of a subject who receives one or more compounds described herein. One or any combination of diagnostic methods, including physical examination, assessment and monitoring of clinical symptoms, and performance of analytical tests can be used for monitoring the health status of a subject. Physical examination can include, for example, determining tenderness, swelling or redness of the affected joint, assessment and monitoring of clinical symptoms. Performance of analytical tests and methods can include, for example, determining the level of inflammatory cytokines or chemokines, X-ray images to determine loss of cartilage as shown by a narrowing of space between the bones in a joint, magnetic resonance imaging (MRI), and providing detailed images of bone and soft tissues. The effects of the treatment of one or more compounds described herein can be analyzed by comparing symptoms of patients suffering from or at risk of an inflammatory disease or disorder, such as osteoarthritis, who have received the treatment with those of patients who have not received such a treatment or who have received a placebo treatment.

In certain embodiments, a compound described herein can be used for treating rheumatoid arthritis (RA). Dysregulation of innate and adaptive immune responses characterize rheumatoid arthritis (RA), and incidence of RA can increase with age. Rheumatoid arthritis is a chronic inflammatory disorder that typically affects the small joints in the hands and feet. Whereas osteoarthritis can result from wear and tear of a joint, rheumatoid arthritis affects the lining of joints, resulting in a painful swelling that can lead to bone erosion and joint deformity. RA can sometimes also affect other organs of the body, such as the skin, eyes, lungs and blood vessels. RA can occur in a subject at any age; however, RA usually begins to develop after age 40. RA can be more prevalent in women.

Chronic inflammation can also contribute to other age-related or aging-related diseases and disorders, such as kyphosis and osteoporosis. Kyphosis is a severe curvature in the spinal column, can be observed with normal and premature aging. Age-related kyphosis often occurs after osteoporosis weakens spinal bones to the point that they crack and compress. A few types of kyphosis target infants or teens. Severe kyphosis can affect lungs, nerves, and other tissues and organs, causing pain and other problems. Characterizing the capability of a compound described herein for treating kyphosis can be determined in pre-clinical animal models. Kyphosis formation is visually measured over time.

In some embodiments, a compound described herein can be used for treating or reducing the likelihood of developing irritable bowel syndrome (IBS) and inflammatory bowel diseases, such as ulcerative colitis and Crohn's disease. Inflammatory bowel disease (IBD) involves chronic inflammation of all or part of the digestive tract. Ulcerative colitis is an inflammatory bowel disease that causes long-lasting inflammation in part of the digestive tract. Symptoms can develop over time, rather than suddenly. Ulcerative colitis usually affects only the innermost lining of the large intestine, colon, and rectum. Crohn's disease is an inflammatory bowel disease that causes inflammation anywhere along the lining of the digestive tract, and often extends deep into affected tissues. The inflammation can lead to abdominal pain, severe diarrhea, and malnutrition. The inflammation caused by Crohn's disease can involve different areas of the digestive tract. Diagnosis and monitoring of the diseases can be accomplished using, for example, blood tests, colonoscopies, flexible sigmoidoscopies, barium enemas, CT scans, MRIs, endoscopies, and small intestine imaging.

In other embodiments, the methods described herein can be useful for treating a subject who has herniated intervertebral discs. Symptoms of a herniated intervertebral disc can include pain, numbness or tingling, or weakness in an arm or leg. Increased levels of pro-inflammatory molecules and matrix metalloproteases (MMPs) can also be found in aging and degenerating discs tissues. Animal models can be used to characterize the effectiveness of a compound described herein in treating herniated intervertebral discs.

Other inflammatory or autoimmune diseases that can be treated by using a compound described herein include eczema, psoriasis, osteoporosis, pulmonary diseases, chronic obstructive pulmonary disease (COPD), idiopathic pulmonary fibrosis (IPF), asthma, inflammatory bowel disease, and mucositis, and oral mucositis, Certain fibrosis or fibrotic conditions of organs such as renal fibrosis, liver fibrosis, pancreatic fibrosis, cardiac fibrosis, skin wound healing, and oral submucous fibrosis can be treated with using a compound described herein.

In certain embodiments, a disease or disorder is an inflammatory disorder of the skin including, for example, psoriasis and eczema that can be treated, or the likelihood reduced, according to the methods described herein that comprise administration of a compound described herein.

Psoriasis is characterized by abnormally excessive and rapid growth of the epidermal layer of the skin. A diagnosis of psoriasis is usually based on the appearance of the skin. Skin characteristics typical for psoriasis are scaly red plaques, papules, or patches of skin that can be painful and itchy. In psoriasis, cutaneous and systemic overexpression of various pro-inflammatory cytokines can be observed. Eczema is an inflammation of the skin that is characterized by redness, skin swelling, itching, dryness, crusting, flaking, blistering, cracking, oozing, or bleeding. One or any combination of diagnostic methods, including physical assessment, monitoring of clinical symptoms, and performance of analytical tests and methods can be used to monitor the effectiveness of compounds described herein. The tests used for monitoring can include, for example, observing the skin's appearance, determining levels of itching, swelling, and pain, and determining the level of pro-inflammatory cytokines.

Other immune disorders or conditions that can be treated or likelihood reduced with a compound described herein include conditions resulting from a host immune response to an organ transplant, for example, kidney, bone marrow, liver, lung, or heart transplant, such as rejection of the transplanted organ. A compound described herein can be used for treating or reducing the likelihood of occurrence of graft-vs-host disease.

### Pulmonary Diseases and Disorders

In some embodiments, methods for treating a disease or disorder comprising administering a compound described herein to a subject in need thereof wherein the disease or disorder is a pulmonary disease or disorder are disclosed. Pulmonary diseases or disorders include, but are not limited to idiopathic pulmonary fibrosis (IPF), chronic obstructive pulmonary disease (COPD), asthma, cystic fibrosis, bronchiectasis, and emphysema.

COPD is a lung disease defined by persistently poor airflow resulting from the breakdown of lung tissue, empysema, and the dysfunction of the small airways, obstructive bronchiolitis. Primary symptoms of COPD include shortness of breath, wheezing, chest tightness, chronic cough, and excess sputum production. Elastase from cigarette smoke-activated neutrophils and macrophages can disintegrate the extracellular matrix of alveolar structures, resulting in enlarged air spaces and loss of respiratory capacity. COPD can be caused by, for example, tobacco smoke, cigarette smoke, cigar smoke, secondhand smoke, pipe smoke, occupational exposure, exposure to dust, smoke, fumes, and pollution, occurring over decades thereby implicating aging as a risk factor for developing COPD.

The processes involved in causing lung damage can include, for example, oxidative stress produced by the high concentrations of free radicals in tobacco smoke, cytokine release due to the inflammatory response to irritants in the airway, and impairment of anti-protease enzymes by tobacco smoke and free radicals, allowing proteases to damage the lungs. Genetic susceptibility can also contribute to the disease. In about 1% percent of people with COPD, the disease results from a genetic disorder that causes low level production of alpha-1-antitrypsin in the liver. Alpha-1-antitrypsin is normally secreted into the bloodstream to help protect the lungs.

Pulmonary fibrosis is a chronic and progressive lung disease characterized by stiffening and scarring of the lung, which can lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis.

Subjects at risk of developing pulmonary fibrosis include, for example, those exposed to environmental or occupational pollutants, such as asbestosis and silicosis; those who smoke cigarettes; those who have a connective tissue diseases such as RA, SLE, scleroderma, sarcoidosis, or Wegener's granulomatosis; those who have infections; those who take certain medications, including, for example, amiodarone, bleomycin, busufan, methotrexate, and nitrofurantoin; those subject to radiation therapy to the chest; and those whose family member have pulmonary fibrosis.

Symptoms of COPD can include any one of shortness of breath, wheezing, chest tightness, having to clear one's throat first thing in the morning because of excess mucus in the lungs, a chronic cough that produces sputum that can be clear, white, yellow or greenish, cyanosis, frequent respiratory infections, lack of energy, and unintended weight loss. Subjects with COPD can also experience exacerbations, during which symptoms worsen and persist for days or longer. Symptoms of pulmonary fibrosis include, for example, shortness of breath, particularly during exercise; dry, hacking cough; fast, shallow breathing; gradual, unintended weight loss; fatigue; aching joints and muscles; and clubbing of the fingers or toes.

The following non-limiting examples of exams or tests can be performed top monitor and assess a subject for a pulmonary disorder: physical exam, determination of patient's medical history, determination of patient's family's medical history, chest X-ray, lung function test, spirometry test, blood test, arterial blood gas analysis, bronchoal veolar lavage, lung biopsy, CT scan, and exercise testing.

Other pulmonary diseases or disorders that can be treated by using a compound described herein include, for example, emphysema, asthma, bronchiectasis, and cystic fibrosis. These diseases can also be exacerbated by tobacco smoke, occupational exposure to dust, smoke, or fumes, infection, or pollutants that contribute to inflammation.

Bronchiectasis can result from damage to the airways that causes them to widen and become flabby and scarred. Bronchiectasis can be caused by a medical condition that injures the airway walls or inhibits the airways from clearing mucus. Examples of such conditions include cystic fibrosis and primary ciliary dyskinesia (PCD). When only one part of the lung is affected, the disorder can be caused by a blockage rather than a medical condition.

The methods described herein for treating or reducing the likelihood of a pulmonary disease or disorder can also be used for treating a subject who is aging and has loss of pulmonary function, or degeneration of pulmonary tissue. The respiratory system can undergo various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract can predispose older adults to increased susceptibility to toxic environmental exposure and accelerated lung function decline. Oxidative stress exacerbates inflammation during aging. Alterations in redox balance and increased oxidative stress during aging precipitate the expression of cytokines, chemokines, and adhesion molecules, and enzymes. Constitutive activation and recruitment of macrophages, T cells, and mast cells foster release of proteases leading to extracellular matrix degradation, cell death, remodeling, and other events that can cause tissue and organ damage during chronic inflammation.

One or any combination of diagnostic methods, including physical examination, assessment and monitoring of clinical symptoms, and performance of analytical tests and methods described herein, can be used for monitoring the health status of the subject. The effects of the treatment of a compound described herein or pharmaceutical composition comprising the agent can be analyzed by, for example, comparing symptoms of patients suffering from or at risk of the pulmonary disease that have received the treatment with those of patients without such a treatment or with placebo treatment. In addition, methods and techniques that evaluate mechanical functioning of the lung, for example, techniques that measure lung capacitance, elastance, and airway hypersensitivity can be performed. To determine lung function and to monitor lung function throughout treatment, any one of numerous measurements can be obtained, for example, expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO₂) can also be measured; normal oxygen levels are typically between 95% and 100%. An SpO₂ level below 90% suggests the subject has hypoxemia. Values below 80% are considered critical and require intervention to maintain brain and cardiac function and avoid cardiac or respiratory arrest.

### Neurological Diseases and Disorders

In some embodiments, methods for treating a disease or disorder comprising administering a compound described herein to a subject in need thereof wherein the disease or disorder is a neurological disease or disorder are disclosed. Neurological diseases or disorders include, but are not limited to Parkinson's disease, Alzheimer's disease, dementia, motor neuron dysfunction (MND), mild cognitive impairment (MCI), Huntington's disease, ocular diseases, age-related macular degeneration, glaucoma, vision loss, presbyopia, and cataracts.

Parkinson's disease (PD) is a disabling condition of the brain characterized by bradykinesia, shaking, stiffness, and loss of balance. Many of these symptoms are due to the loss of certain nerves in the brain, which results in the lack of dopamine. This disease is characterized by neurodegeneration of the dopaminergic neurons in the substantia nigra pars compacta, a profound loss of dopamine in the striatum, and/or the presence of intracytoplasmic inclusions (Lewy bodies), which are composed mainly of alpha-synuclein and ubiquitin. Parkinson's disease also features locomotor deficits, such as tremor, rigidity, bradykinesia, and/or postural instability. Subjects at risk of developing Parkinson's disease include those having a family history of Parkinson's disease and those exposed to pesticides, for example, rotenone or paraquat), herbicides, for example, agent orange, or heavy metals.

Methods for detecting, monitoring or quantifying neurodegenerative deficiencies or motor deficits associated with Parkinson's diseases include, for example, histological studies, biochemical studies, and behavioral assessments. Symptoms of Parkinson's disease include, but are not limited to, difficulty starting or finishing voluntary movements, jerky, stiff movements, muscle atrophy, tremors, changes in heart rate, normal reflexes, bradykinesia, and postural instability. There is a growing recognition that people diagnosed with Parkinson's disease can have cognitive impairment, including mild cognitive impairment, in addition to their physical symptoms.

Alzheimer's disease (AD) is a neurodegenerative disease that shows progressive mental deterioration with failure of memory, disorientation, and confusion, leading to profound dementia. Age is an important predisposing risk factor for developing AD, which is a major cause of dementia in the elderly. Early clinical symptoms show remarkable similarity to mild cognitive impairment. As the disease progresses, impaired judgment, confusion, behavioral changes, disorientation, difficulty walking, and difficulty swallowing occur.

AD is characterized by the presence of neurofibrillary tangles and amyloid plaques in histological specimens. AD involves the limbic and cortical regions of the brain. The argyrophilic plaques containing the amyloidogenic Aβ fragment of amyloid precursor protein (APP) are scattered throughout the cerebral cortex and hippocampus. Neurofibrillary tangles are found in pyramidal neurons predominantly located in the neocortex, hippocampus, and nucleus basalis of Meynert. Other changes, such as granulovacuolar degeneration in the pyramidal cells of the hippocampus, and neuron loss and gliosis in the cortex and hippocampus, are observed. Subjects at risk of developing AD include those of advanced age, those with a family history of AD, those with genetic risk genes, for example, ApoE4, or deterministic gene mutations, for example, APP, PS1, or PS2, and those with a history of head trauma or heart/vascular conditions, for example, high blood pressure, heart disease, stroke, diabetes, high cholesterol.

A number of behavioral and histopathological assays can be used for evaluating the Alzheimer's disease phenotype, for characterizing therapeutic agents, and assessing treatment for AD. Histological analyses are typically performed postmortem. Histological analysis of Aβ levels can be performed using Thioflavin-S, Congo red, or anti-Aβ staining. In vivo methods of visualizing Aβ deposition in transgenic mice can also be performed. BSB ((trans, trans)-1-bromo-2,5-bis-(3-hydroxycarbonyl-4-hydroxy)styrylbenzene) and PET tracer ¹¹C-labelled Pittsburgh Compound-B (PIB) bind to Aβ plaques. ¹⁹F-containing amyloidophilic, Congo red-type compound FSB ((E,E)-1-fluoro-2,5-bis-(3-hydroxycarbonyl-4-hydroxy)styrylbenzene) allows visualization of Aβ plaques by MRI. Radiolabeled, putrescine-modified amyloid-beta peptide labels amyloid can deposit in vivo in a mouse model of Alzheimer's disease.

Increased glial fibrillary acidic protein (GFAP) by astrocytes is a marker for astroglial activation and gliosis during neurodegeneration. Aβ plaques are associated with GFAP-positive activated astrocytes, and can be visualized via GFAP staining. Neurofibrillary tangles can be identified by immunohistochemistry using thioflavin-S fluorescent microscopy and Gallyas silver stains. Axon staining with electron microscopy and axonal transport studies can be used to neuronal degeneration.

Diagnosis of Alzheimer's disease can be based on, for example, progressive decline in memory function, gradual retreat from and frustration with normal activities, apathy, agitation or irritability, aggression, anxiety, sleep disturbance, dysphoria, aberrant motor behavior, disinhibition, social withdrawal, decreased appetite, hallucinations, dementia, medical history, neuropsychological tests, and neurological or physical examination of a patient. Cerebrospinal fluid can also be for testing for various proteins that are associated with Alzheimer pathology, including tau, amyloid beta peptide, and AD7C-NTP. Genetic testing can also be used for early-onset familial Alzheimer disease (eFAD), an autosomal-dominant genetic disease.

One or any combination of diagnostic methods, including physical examination, assessment and monitoring of clinical symptoms, and performance of analytical tests and methods described herein, can be used for monitoring the health status of the subject. The effects of administering one or more compounds described herein can be analyzed by, for example, comparing symptoms of patients suffering from or at risk of AD that have received the treatment with those of patients without such a treatment or with placebo treatment.

Mild Cognitive Impairment (MCI) is a brain-function syndrome involving the onset and evolution of cognitive impairments beyond those expected based on age and education of the individual, but which are not significant enough to interfere with this individual's daily activities. MCI is an aspect of cognitive aging that is considered to be a transitional state between normal aging and the dementia. MCI that primarily affects memory is known as “amnestic MCI.” A person with amnestic MCI can start to forget important information that he or she would previously have recalled easily, such as recent events. Amnestic MCI is frequently seen as prodromal stage of Alzheimer's disease. MCI that affects thinking skills other than memory is known as “non-amnestic MCI.” Non-amnestic MCI can affect thinking skills such as the ability to make sound decisions, judge the time or sequence of steps needed to complete a complex task, or visual perception. Individuals with non-amnestic MCI are believed to be more likely to convert to other types of dementias, for example, dementia with Lewy bodies.

People diagnosed with Parkinson's disease can have MCI in addition to their physical symptoms. Parkinson's disease patients with MCI sometimes go on to develop dementia.

Methods for detecting, monitoring, quantifying or assessing neuropathological deficiencies associated with MCI can include, for example, astrocyte morphological analyses, release of acetylcholine, silver staining for assessing neurodegeneration, and PiB PET imaging to detect beta amyloid deposits. Methods for detecting, monitoring, quantifying or assessing behavioral deficiencies associated with MCI can include, for example, eight-arm radial maze paradigm, non-matching-to-sample task, allocentric place determination task in a water maze, Morris maze test, visuospatial tasks, and delayed response spatial memory task, and olfactory novelty test.

Motor Neuron Dysfunction (MND) is a group of progressive neurological disorders that destroy motor neurons, the cells that control essential voluntary muscle activity such as speaking, walking, breathing and swallowing. MNDs are classified according to whether degeneration affects upper motor neurons, lower motor neurons, or both. Examples of MNDs include, but are not limited to, Amyotrophic Lateral Sclerosis (ALS), Lou Gehrig's Disease, progressive bulbar palsy, pseudobulbar palsy, primary lateral sclerosis, progressive muscular atrophy, lower motor neuron disease, and spinal muscular atrophy (SMA), Werdnig-Hoffmann Disease (SMA1), SMA2, Kugelberg-Welander Disease (SM3), Kennedy's disease, post-polio syndrome, and hereditary spastic paraplegia.

ALS can affect the arms, legs, or facial muscles. Primary lateral sclerosis is a disease of the upper motor neurons, while progressive muscular atrophy affects only lower motor neurons in the spinal cord. In progressive bulbar palsy, the lowest motor neurons of the brain stem are most affected, causing slurred speech and difficulty chewing and swallowing. Patients with MND can exhibit, for example, tremors, rigidity, bradykinesia, or postural instability.

Methods for detecting, monitoring, quantifying, or assessing motor deficits and histopathological deficiencies associated with MND can include, for example, histopathological, biochemical, and electrophysiological studies, and motor activity analysis. Histopathologically, MNDs can be characterized by death of motor neurons, progressive accumulation of detergent-resistant aggregates containing SOD1, and ubiquitin and aberrant neurofilament accumulations in degenerating motor neurons. In addition, reactive astroglia and microglia can be detected in diseased tissue. Patients with an MND show one or more motor deficits, including muscle weakness and wasting, uncontrollable twitching, spasticity, slow and effortful movements, and overactive tendon reflexes.

Opthalmic diseases and disorders include, but are not limited to, presbyopia, macular degeneration, or cataracts. In other certain embodiments, the disease or disorder is glaucoma.

Macular degeneration is a neurodegenerative disease that causes the loss of photoreceptor cells in the central part of retina, called the macula. Macular degeneration can be dry or wet. The dry form is more common than the wet, with about 90% of age-related macular degeneration (ARMD or AMD) patients diagnosed with the dry form. The wet form of the disease can lead to more serious vision loss. Age and certain genetic factors and environmental factors can be risk factors for developing ARMD. Environmental factors include, for example, omega-3 fatty acids intake, estrogen exposure, and increased serum levels of vitamin D Genetic risk factors can include, for example, reduced ocular Dicer1 levels, and decreased micro RNAs, and DICER1 ablation.

Dry ARMD is associated with atrophy of the retinal pigment epithelium (RPE) layer, which causes loss of photoreceptor cells. The dry form of ARMD can result from aging and thinning of macular tissues and from deposition of pigment in the macula. With wet ARMD, new blood vessels can grow beneath the retina and leak blood and fluid. Abnormally leaky choroidal neovascularization can cause the retinal cells to die, creating blind spots in central vision. Different forms of macular degeneration can also occur in younger patients. Non-age related etiology can be linked to, for example, heredity, diabetes, nutritional deficits, head injury, or infection.

The formation of exudates, or “drusen,” underneath the Bruch's membrane of the macula is can be a physical sign that macular degeneration can develop. Symptoms of macular degeneration include, for example, perceived distortion of straight lines and, in some cases, the center of vision appears more distorted than the rest of a scene; a dark, blurry area or “white-out” appears in the center of vision; or color perception changes or diminishes.

Presbyopia is an age-related condition where the eye exhibits a progressively diminished ability to focus on near objects as the speed and amplitude of accommodation of a normal eye decreases with advancing age. Loss of elasticity of the crystalline lens and loss of contractility of the ciliary muscles can cause presbyopia. Age-related changes in the mechanical properties of the anterior lens capsule and posterior lens capsule suggest that the mechanical strength of the posterior lens capsule decreases significantly with age.

The laminated structure of the capsule of the eye also changes and can result, at least in part, from a change in the composition of the tissue. The major structural component of the lens capsule is basement membrane type IV collagen that is organized into a three-dimensional molecular network. Type IV collagen is composed of six homologous α chains (α1-6) that associate into heterotrimeric collagen IV protomers with each comprising a specific chain combination of α112, α345, or α556. Protomers share structural similarities of a triple-helical collagenous domain with the triplet peptide sequence of Gly-X-Y, ending in a globular C-terminal region termed the non-collagenous 1 (NC1) domain. The N-termini are composed of a helical domain termed the 7S domain, which is also involved in protomer-protomer interactions.

Collagen IV can influence cellular function and tissue stabilization. Posterior capsule opacification (PCO) develops as a complication in approximately 20-40% of patients in subsequent years after cataract surgery. PCO results from proliferation and activity of residual lens epithelial cells along the posterior capsule in a response akin to wound healing. Growth factors, such as fibroblast growth factor, transforming growth factor (3, epidermal growth factor, hepatocyte growth factor, insulin-like growth factor, and interleukins IL-1 and IL-6, can also promote epithelial cell migration. In vitro, collagen IV can promote adherence of lens epithelial cells. Adhesion of the collagen IV, fibronectin, and laminin to the intraocular lens can inhibit cell migration and can reduce the risk of PCO.

Compounds described herein can slow the disorganization of the type IV collagen network, decrease or inhibit epithelial cell migration and can also delay the onset of presbyopia or decrease or slow the progressive severity of the condition. The compounds described herein can also be useful for post-cataract surgery to reduce the likelihood of occurrence of PCO.

Cataracts are a clouding of the lens of an eye, causing blurred vision, and if left untreated can result in blindness. Surgery is effective and routinely performed to remove cataracts. Administration of one or more of the compounds described herein can result in decreasing the likelihood of occurrence of a cataract.

In some embodiments, a compound is administered to a subject who is at risk of developing presbyopia, cataracts, or macular degeneration. Treatment with a compound described herein can be initiated when a human subject is at least 40 years of age to delay or inhibit onset or development of cataracts, presbyopia, and macular degeneration.

In some embodiments, the disease or disorder is glaucoma. Normally, clear fluid flows into and out of the front part of the eye, known as the anterior chamber. In individuals who have open/wide-angle glaucoma, the clear fluid drains too slowly, leading to increased pressure within the eye. If left untreated, the high pressure in the eye can subsequently damage the optic nerve and can lead to complete blindness. The loss of peripheral vision is caused by the death of ganglion cells in the retina.

Non-limiting examples for monitoring the effect of a therapy on inhibiting progression of glaucoma include automated perimetry, gonioscopy, imaging technology, scanning laser tomography, HRT3, laser polarimetry, GDX, ocular coherence tomography, ophthalmoscopy, and pachymeter measurements that determine central corneal thickness.

### Metabolic Diseases and Disorders

In some embodiments, methods for treating a disease or disorder comprising administering a compound described herein to a subject in need thereof wherein the disease or disorder is a metabolic disease or disorder are disclosed. Metabolic diseases or disorders include, but are not limited to diabetes, metabolic syndrome, diabetic ulcers, and obesity.

Diabetes is characterized by high levels of blood glucose caused by defects in insulin production, insulin action, or both. Type 2 diabetes can be characterized by the gradual loss of insulin production by the pancreas. Diabetes can cause kidney failure, nontraumatic lower-limb amputations, and blindness. Diabetes can cause heart disease and stroke. A compound described herein can be used for treating type 2 diabetes, particularly age-, diet- and obesity-associated type 2 diabetes.

Diagnosis of type 2 diabetes can be based on symptoms, medical history, or physical examination of a patient. Symptoms of a patient with type 2 diabetes can include, for example, increased thirst and frequent urination, increased hunger, weight loss, fatigue, blurred vision, slow-healing sores or frequent infections, or areas of darkened skin. Subjects at risk of developing type 2 diabetes include those who have a family history of type 2 diabetes and those who have other risk factors such as excess weight, fat distribution, inactivity, race, age, prediabetes, or gestational diabetes.

One or any combination of diagnostic methods, including physical examination, assessment and monitoring of clinical symptoms, and performance of analytical tests and methods, such as those described herein, can be used for monitoring the health status of the subject. A subject who is receiving one or more compounds described herein for treatment or reduction in the likelihood of developing diabetes can be monitored, for example, by assaying glucose and insulin tolerance, energy expenditure, body composition, fat tissue, skeletal muscle, and liver inflammation, or lipotoxicity.

Obesity and obesity-related disorders can refer to conditions of subjects who have a body mass that is measurably greater than ideal for their height and frame. Body Mass Index (BMI) is a measurement tool used to determine excess body weight, and is calculated from the height and weight of a subject. A human is considered overweight when the person has a BMI of 25-29; a person is considered obese when the person has a BMI of 30-39, and a person is considered severely obese when the person has a BMI of ≧40. Accordingly, the terms obesity and obesity-related refer to human subjects with body mass index values of greater than 30, greater than 35, or greater than 40. A category of obesity not captured by BMI is called “abdominal obesity”, which relates to the extra fat found around a subject's middle, which is an important factor in health, independent of BMI. Abdominal obesity in women can be a waist size 35 inches or higher, and in men a waist size of 40 inches or higher.

In some embodiments, the disease or disorder is a diabetic ulcer. An ulcer is a breakdown in the skin, which can extend to involve the subcutaneous tissue or even muscle or bone. These lesions occur, particularly, on the lower extremities. Chronic inflammation is observed at sites of chronic wounds, such as diabetic ulcers.

Subjects who have type 2 diabetes or who are at risk of developing type 2 diabetes can have metabolic syndrome. Metabolic syndrome in humans can be associated with obesity and characterized by one or more of cardiovascular disease, liver steatosis, hyperlipidemia, diabetes, and insulin resistance. A subject with metabolic syndrome can present with a cluster of metabolic disorders or abnormalities which can include, for example, one or more of hypertension, type-2 diabetes, hyperlipidemia, dyslipidemia, hypertriglyceridemia, hypercholesterolemia, insulin resistance, liver steatosis, steatohepatitis, hypertension, or atherosclerosis.

In some embodiments, the metabolic disease or disorder comprises renal dysfunction. Nephrological pathologies, such as glomerular disease, can arise in the elderly. Glomerulonephritis is characterized by inflammation of the kidney and by the expression of proteins, for example, IL1α and IL1β. In some embodiments, the metabolic disease or disorder is glomerular disease.

### Dermatological Diseases and Disorders

In some embodiments, methods for treating a disease or disorder comprising administering a compound described herein to a subject in need thereof wherein the disease or disorder is a dermatological disease or disorder are disclosed. Such dermatological diseases or disorders can include psoriasis and eczema. Other dermatological diseases and disorders include rhytides, pruritis, dysesthesia, papulosquamous disorders, erythroderma, lichen planus, lichenoid dermatosis atopic dermatitis, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus. Late onset lupus can be linked to decreased function of T-cells and B-cells and cytokines associated with aging.

### Metastasis

In some embodiments, methods for treating a disease or disorder comprising administering a compound described herein to a subject in need thereof wherein the disease or disorder comprises a metastasis are disclosed. In some embodiments, a compound described herein is administered to a subject who has a cancer. Metastasis of a cancer occurs when the cancer cells spread beyond the anatomical site of origin and initial colonization to other areas throughout the body of the subject. Tumor proliferation can be determined by tumor size, which can be measured in various ways, for example, PET scanning, MRI, CAT scan, or biopsy. The effect of the therapeutic agent on tumor proliferation can also be evaluated by examining differentiation of the tumor cells.

Cancers or tumors can be characterized by cells exhibiting abnormal cellular proliferation. Cancer can describe a malignant tumor or the disease state arising from the tumor. Alternatively, an abnormal growth can be a neoplasm. A tumor, such as in reference to a tissue, can be abnormal tissue growth that can be characterized by excessive and abnormal cellular proliferation. A tumor can be metastatic and capable of spreading beyond the anatomical site of origin and initial colonization to other areas throughout the body of the subject. A cancer can comprise a solid tumor or can comprise a “liquid” tumor, for example, leukemia.

In one embodiment, methods are provided for reducing the likelihood of metastasis in a subject who has a cancer by administering a compound as described herein. In a particular embodiment, the compound is administered on one or more days within a treatment window. In other embodiments, the treatment course is 1, 2, 3, 4, 5, 6, 7, 8, 9, 10, 11, 12, 13, 14, 15, 16, 17, 18, 19, 20, or 21 days. In certain embodiments, the compound is administered on two or more days within a treatment window of no longer than 7 days or 14 days, on 3 or more days within a treatment window of no longer than 7 days or 14 days; on 4 or more days within a treatment window of no longer than 7 days or 14 days; on 5 or more days within a treatment window of no longer than 7 days or 14 days; on 6, 7, 8, 9, 10, 11, 12, 13, or 14 days within treatment window of no longer than 7 days or 14 days. In some embodiments, when the at least one compound is administered to a subject for a treatment window of 3 days or more, the agent can be administered every 2^{nd} day. In some embodiments when at least one compound is administered to a subject for a treatment window of 4 days or more, the agent can be administered every 3^{rd} day.

Many chemotherapy and radiotherapy treatment regimens comprise a finite number of cycles of on-drug therapy followed by off-drug therapy or comprise a finite timeframe in which the chemotherapy or radiotherapy is administered. The protocols are determined by clinical trials, drug labels, and clinical staff in conjunction with the subject to be treated. The number of cycles of a chemotherapy or radiotherapy or the total length of time of a chemotherapy or radiotherapy regimen can vary depending on the patient's response to the cancer therapy. In another embodiment for treating metastasis, a compound can be administered after the treatment regimen of chemotherapy or radiotherapy has been completed.

A cancer that can metastasize can be a solid tumor or can be a liquid tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In certain specific embodiments, the disease or disorder treated or the likelihood reduced by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, and kidney cancer cells.

The methods described herein can also be used for inhibiting progression of metastatic cancer tumors. Non-limiting types of cancers include the following: adrenocortical carcinoma, childhood adrenocortical carcinoma, aids-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, aids-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenström macroglobulinemia.

### Chemotherapy and Radiotherapy Side Effects

In some embodiments, methods for treating a disease or disorder comprising administering a compound described herein to a subject in need thereof wherein the disease or disorder comprises a chemotherapy-induced or radiotherapy-induced side effect are disclosed. Examples of chemotherapeutic agents include, for example, anthracyclines, doxorubicin, daunorubicin, taxols, paclitaxel, gemcitabine, pomalidomide, and lenalidomide. One or more of the compound described herein can be used for treating or reducing the likelihood of a chemotherapeutic side effect or a radiotherapy side effect. Acute toxic side effects include but are not limited to gastrointestinal toxicity, nausea, vomiting, constipation, anorexia, diarrhea, peripheral neuropathy, fatigue, malaise, low physical activity, hematological toxicity, anemia, hepatotoxicity, alopecia, pain, infection, mucositis, fluid retention, dermatological toxicity, rashes, dermatitis, hyperpigmentation, urticaria, photosensitivity, nail changes, mouth, gum or throat problems, or any toxic side effect caused by a chemotherapy or radiotherapy. For example, toxic side effects caused by radiotherapy or chemotherapy can be ameliorated by the methods described herein. Accordingly, in certain embodiments, methods are provided herein for ameliorating acute toxicity or reducing severity of a toxic side effect of a chemotherapy or radiotherapy or both in a subject who receives the therapy, wherein the method comprises administering to the subject a compound described herein. As described for treating or reducing the likelihood of metastasis, the compound is administered during the off-chemotherapy or off-radiotherapy time interval or after the chemotherapy or radiotherapy treatment regimen has been completed.

In some embodiments, the acute toxicity is an acute toxicity comprising energy imbalance and can comprise one or more of weight loss, endocrine changes, hormone imbalance, changes in hormone signaling, and changes in body composition. In certain embodiments, an acute toxicity comprising energy imbalance relates to decreased or reduced ability of the subject to be physically active, as indicated by decreased or diminished expenditure of energy than would be observed in a subject who did not receive the medical therapy. By way of non-limiting example, such an acute toxic effect that comprises energy imbalance includes low physical activity. In some embodiments, energy imbalance comprises fatigue or malaise.

In one embodiment, a chemotherapy side effect to be treated or the likelihood reduced by a compound described herein is cardiotoxicity. A subject who has a cancer that is being treated with an anthracycline can be treated with one or more compounds described herein that reduce the cardiotoxicity of the anthracycline. Administration of one or more of the compounds described herein can reduce the cardiotoxicity such that additional amounts of the anthracycline can be administered to the subject, resulting in an improved prognosis related to cancer disease. In one embodiment, the cardiotoxicity results from administration of an anthracyline, such as doxorubicin. Doxorubicin is an anthracycline topoisomerase that is approved for treating patients who have, for example, ovarian cancer after failure of a platinum based therapy, Kaposi's sarcoma after failure of primary systemic chemotherapy or intolerance to the therapy, or multiple myeloma in combination with bortezomib in patients who have not previously received bortezomib or who have received at least one prior therapy. Doxorubicin can cause myocardial damage that could lead to congestive heart failure if the total lifetime dose to a patient exceeds 550 mg/m². Cardiotoxicity can occur at even lower doses if the patient also receives mediastinal irradiation or another cardiotoxic drug.

In other embodiments, a compound described herein can be used in the methods as provided herein for ameliorating chronic or long term side effects. Chronic toxic side effects typically result from multiple exposures to or administrations of a chemotherapy or radiotherapy over a longer period of time. Certain toxic effects appear long after treatment and result from damage to an organ or system by the therapy. Organ dysfunction, for example, neurological, pulmonary, cardiovascular, and endocrine dysfunction, can be observed in patients who were treated for cancers during childhood. Chronic or late toxic side effects that occur in subjects who received chemotherapy or radiation therapy include by way of non-limiting example, cardiomyopathy, congestive heart disease, inflammation, early menopause, osteoporosis, infertility, impaired cognitive function, peripheral neuropathy, secondary cancers, cataracts and other vision problems, hearing loss, chronic fatigue, reduced lung capacity, and lung disease.

### Age-Related Diseases and Disorders

In some embodiments, methods for extending the lifespan of a subject comprising administering to the subject a compound described herein are disclosed. In some embodiments, a compound described herein is used to treat or reduce a likelihood of developing an age-related disease or disorder that occurs as part of the natural aging process or that occurs when the subject is exposed to an agent or factor such as irradiation, chemotherapy, smoking tobacco, high-fat/high sugar diet, other environmental factors. Age related diseases or conditions include, for example, renal dysfunction, kyphosis, herniated intervertebral disc, frailty, hair loss, hearing loss, vision loss, muscle fatigue, skin conditions, skin nevi, diabetes, metabolic syndrome, and sarcopenia.

Vision loss can be an absence of vision when a subject previously had vision. Various scales can be used to describe the extent of vision and vision loss based on visual acuity. Age-related diseases and conditions also include dermatological conditions, for example without limitation, treating one or more of the following conditions: wrinkles, including superficial fine wrinkles; hyperpigmentation; scars; keloid; dermatitis; psoriasis; eczema, seborrheic eczema; rosacea; vitiligo; ichthyosis vulgaris; dermatomyositis; and actinic keratosis.

Frailty can be an increased vulnerability resulting from aging-associated decline in reserve and function across multiple physiologic systems that compromise a subject's ability to cope with every day or acute stressors. Frailty can be characterized by compromised energy characterized by, for example, low grip strength, low energy, slowed waking speed, low physical activity, or unintentional weight loss. A patient can be diagnosed with frailty when three of five of the foregoing characteristics are observed. In certain embodiments, aging and diseases and disorders related to aging can be treated or the likelihood reduced by administering a compound described herein.

One or any combination of diagnostic methods appropriate for the particular disease or disorder, including physical examination, patient self-assessment, assessment and monitoring of clinical symptoms, performance of analytical tests and methods, including clinical laboratory tests, physical tests, and exploratory surgery, for example, can be used for monitoring the health status of the subject and the effectiveness of a compound disclosed herein. The effects of the methods of treatment described herein can be analyzed by, for example, comparing symptoms of patients suffering from or at risk of a particular disease or disorder that have received the pharmaceutical composition comprising a compound described herein with those of patients who were not treated with the compound or who received a placebo treatment.

### Erdheim-Chester Disease

In some embodiments, methods for treating a disease or disorder comprising administering a compound described herein to a subject in need thereof wherein the disease or disorder is Erdheim-Chester Disease are disclosed.

Erdheim-Chester disease (ECD) (also known as Erdheim-Chester syndrome or polyostotic sclerosing histiocytosis) is a rare disease characterized by the abnormal multiplication of a specific type of white blood cells called histiocytes, or tissue macrophages. Usually, onset of ECD is in middle age. ECD involves an infiltration of lipid-laden macrophages, multinucleated giant cells, an inflammatory infiltrate of lymphocytes and histiocytes in the bone marrow, and a generalized sclerosis of the long bones.

Long bone involvement is almost universal in ECD patients and is bilateral and symmetrical in nature. More than 50% of cases have some sort of extraskeletal involvement. Extraskeletal involvement can include, for example, kidney, skin, brain, lung involvement, retroorbital tissue, pituitary gland, and heart involvement.

Bone pain can be a symptom associated with ECD and can affect the lower limbs, knees and ankles. Exophthalmos can occur in some patients and can be bilateral, symmetric and painless, and can occur several years before the final diagnosis. Recurrent pericardial effusion can be a manifestation, as can morphological changes in adrenal size and infiltration. Symptoms that accompany ECD can include, for example, bone pain, retroperitoneal fibrosis, diabetes insipidus, exophthalmos, xanthomas, neurological signs, ataxia, dyspnea caused by interlobular septal and pleural thickening, kidney failure, hypopituitarism, and liver failure.

Radiologic osteosclerosis and histology can be diagnostic features for ECD. Video-assisted thoracoscopic surgery can be used for diagnostic confirmation and also for therapeutic relief of recurrent pericardial fluid drainage.

### Premature Aging Diseases and Disorders

In some embodiments, methods for treating a disease or disorder comprising administering a compound described herein to a subject in need thereof wherein the disease or disorder comprises a premature aging disease or disorder are disclosed. In some embodiments, the premature aging disease or disorder is Hutchinson-Gilford progeria or Werner's Syndrome.

Progeria (Hutchinson-Gilford progeria syndrome, HGPS, progeria syndrome) is a genetic disorder wherein symptoms resembling aspects of aging are manifested at a very early age. Progeria is one of several progeroid syndromes. Those born with progeria can have shortened lifespans.

Children with progeria can develop the first symptoms during the first few months of life. The earliest symptoms can include, for example, a failure to thrive and a localized scleroderma-like skin condition. As a child ages past infancy, additional conditions can become apparent. Limited growth, full-body alopecia, and a small face with a shallow recessed jaw, and a pinched nose, can be characteristics of progeria. Later, progeria can cause wrinkled skin, atherosclerosis, kidney failure, loss of eyesight, and cardiovascular problems. Scleroderma, a hardening and tightening of the skin on trunk and extremities of the body, can be prevalent. People diagnosed with progeria can have small, fragile bodies. The face of a subject with progeria can be wrinkled, with a larger head in relation to the body, a narrow face and a beak nose. Prominent scalp veins are noticeable, as well as prominent eyes. Musculoskeletal degeneration can cause loss of body fat and muscle, stiff joints, and hip dislocations. Individuals with progeria can retain normal mental and motor development.

In normal conditions, the LMNA gene codes for a structural protein called prelamin A. There is a farnesyl functional group attached to the carboxyl-terminus of the structure of prelamin A. The farnesyl group allows prelamin A to attach temporarily to the nuclear rim. Once prelamin A is attached, the farnesyl group is removed. Failure to remove this farnesyl group permanently affixes prelamin A to the nuclear rim. Without the farnesyl group, prelamin A is referred to as lamin A. Lamin A, along with lamin B and lamin C, makes up the nuclear lamina, which can provide structural support to the nucleus.

The cause of progeria is a point mutation at position 1824 of the LMNA gene, in which a cytosine is replaced with thymine. This point mutation creates a 5′ cryptic splice site within exon 11, resulting in an abnormally short mature mRNA transcript. This mRNA strand, when translated, yields an abnormal variant of the prelamin A protein whose farnesyl group cannot be removed. As the farnesyl group cannot be removed, this abnormal prelamin A, referred to as progerin, is permanently affixed to the nuclear rim, and therefore does not become part of the nuclear lamina Without lamin A, the nuclear lamina is unable to provide the nuclear envelope with adequate structural support, and an abnormal shape results. Since the support that the nuclear lamina normally provides is necessary for the organizing of chromatin during mitosis, weakening of the nuclear lamina limits the ability of the cell to divide.

To date, over 1,400 SNPs of LMNA gene are known. They can manifest in changes on mRNA, splicing or protein, for example mutations such as Arg471Cys, g482Gln, Arg527Leu, Arg527Cys, Ala529Val, level. A genetic test for LMNA mutations can confirm the diagnosis of progeria.

Werner syndrome (WS), also known as “adult progeria”, is an autosomal recessive progeroid syndrome (PS), which can be characterized by the appearance of premature aging. Affected individuals typically grow and develop normally until puberty, and the mean age of diagnosis is twenty-four, often realized when the adolescent growth spurt is not observed. The median and mean ages of death are 47-48 and 54 years, respectively. A major cause of death due to Werner Syndrome is cardiovascular disease or cancer.

Werner Syndrome patients can exhibit, for example, growth retardation, short stature, premature graying of hair, alopecia, wrinkling, prematurely aged faces with beaked noses, skin atrophy, scleroderma-like lesions, lipodystrophy, abnormal fat deposition leading to thin legs and arms, and severe ulcerations around the Achilles tendon and malleoli. Other symptoms can include, for example, change in voice (weak, hoarse, high-pitched), atrophy of gonads leading to reduced fertility, bilateral cataracts, premature arteriosclerosis, calcinosis, atherosclerosis, type 2 diabetes, osteoporosis, telangiectasia, and malignancies. The prevalence of rare cancers, for example, meningiomas, are increased in individuals with Werner syndrome.

The WRN mutation that causes Werner syndrome is autosomal and recessive. Patients with Werner syndrome can display rapid premature aging beginning in young adulthood, usually in their early twenties. Diagnosis can be based on six symptoms: premature graying of the hair or hair loss, presence of bilateral cataracts, atrophied or tight skin, soft tissue calcification, sharp facial features, and an abnormal, high-pitched voice. Werner syndrome patients can also be short-statured due to absence of the adolescent growth spurt that usually occurs during puberty. Patients with Werner syndrome can display decreased fertility.

Werner syndrome patients can have skin that appears shiny and tight, and can also be thin or hardened. The skin phenotype can be due to atrophy of the subcutaneous tissue and dermal fibrosis. Over time, the features of Werner syndrome patients can be more apparent due to skin conditions. Other associated skin conditions include, for example, ulcers, which can be caused by a decreased potential of skin cells for replication.

The cataracts seen in patients with Werner Syndrome are distinctly different from those that can develop with normal aging. The cataracts can be associated with problems in the lens posterior cortex and subcapsular regions. These cataracts are generally treatable with cataract surgery, which should restore normal vision for Werner syndrome patients.

Werner syndrome patients can be at increased risk for several other diseases, many associated with aging including, for example, atherosclerosis, nervous system disorders, osteoporosis, diabetes mellitus, and skin ulcers.

Werner syndrome patients can also be at an increased risk of cancer, especially malignant melanoma. Soft-tissue sarcomas are the most common types of cancer experienced by Werner syndrome patients. Other types of skin cancer, other epithelial cancers such as thyroid and liver cancers, MDS (myelodysplastic syndrome), and MFH (malignant fibrous histiocytoma) are also prevalent among Werner syndrome patients. Mutations in the WRN gene, especially single-nucleotide polymorphisms (SNPs), can be associated with many of the cancers and other associated diseases of Werner syndrome. WRN SNPs can correlate with cancers such as sarcomas and non-Hodgkin lymphomas, as well as diabetes and cardiovascular problems including atherosclerosis.

WRN, which lies on chromosome 8 in humans, encodes the WRNp protein, a 1432 amino acid protein with a central domain resembling members of the RecQ helicases. RecQ helicases are a special type of helicase that function at unique times during DNA repair of doubled stranded breaks, which are a form of DNA damage that results in a break of both strands of DNA. Thus, RecQ helicases are important for maintaining DNA stability, and loss of function of these helicases can have important implications in the development of Werner syndrome. In addition to the central domain, there are three exonucleus domains in the N-terminus region and a ribonucleas helicase D localized in the C-terminus region.

When functioning normally, the WRN gene and associated protein are important for maintaining genome stability. WRNp is active in unwinding DNA, a step necessary in DNA repair and DNA replication. Specifically, the WRN protein can have an important role in responding to replication malfunctions, particularly double-stranded breaks, and stalled replication machinery. WRN can reactivate replication by reducing a likelihood of developing unwanted recombination processes from occurring or by promoting recombination, depending on the type of DNA damage. In addition, the WRN protein physically interacts with or binds to several other proteins that are involved in processing DNA. For example, the WRN protein binds to replication protein A (RPA), which can stimulate WRNp's helicase activity. WRNp also physically interacts with p53, a tumor suppressor gene that stops the formation of tumors and the progression of cancers, which inhibits the exonuclease activity of the WRNp. Since WRNp's function depends on DNA, WRNp is only functional when localized to the nucleus.

Mutations which cause Werner syndrome all occur at the regions of the gene which encode for protein, and not at non-coding regions. There are 35 different known mutations of WRN, which correspond to stop codons, insertions, or deletions that result in a frameshift mutation. These mutations can have a range of effects. For example, the mutations can decrease the stability of the transcribed mRNA, which increases the rate at which they are degraded. With less mRNA, less is available to be translated into the WRNp protein. Mutations can also lead to the truncation (shortening) of the WRNp protein, leading to the loss of the nuclear localization signal sequence, thus the protein is no longer transported into the nucleus to interact with the DNA. This action leads to a reduction in DNA repair. Furthermore, mutated proteins are more likely to be degraded than normal WRNp. Apart from causing defects in DNA repair, the aberrant association with p53 down-regulates the function of p53, leading to a reduction in p53-dependent apoptosis and increasing the survival of these dysfunctional cells. Cells of affected individuals also have reduced lifespan in culture, have more chromosome breaks and translocations and have extensive deletions.

Patients with Werner syndrome lose the RecQ helicase activity in the WRN protein because of the loss of the C-terminus region. The loss of the helicase activity can have far-reaching consequences in terms of cell stability and mutation. One instance of these consequences involves telomeres. The WRN helicase activity is important not only for DNA repair and recombination, but also for maintaining telomere length and stability. Thus, WRN helicase is important for reducing a likelihood of developing catastrophic telomere loss during DNA replication. In a normal cell, the telomeres undergo repeated shortening during the cell cycle, which can reduce the likelihood of the cell from dividing and multiplying. This event can be counteracted by telomerase, an enzyme that extends the ends of the chromosomes by copying the telomeres and synthesizing an identical, but new end that can be added to the existing chromosome. However, patients with Werner syndrome often exhibit accelerated telomere shortening, indicating that there can be a connection between the loss of the WRN helicase activity and telomere and cell instability.

Without the WRN protein, the interwoven pathways of DNA repair and telomere maintenance fail to suppress cancer and the aging symptoms seen in patients with WS. Events such as rapid telomere shortening cause Werner syndrome cells to exhibit low responses to overall cellular stress. In addition to telomere dysfunction, over-expression of oncogenes and oxidation can induce this type of response. High stress causes a synergistic effect, where WS cells become even more sensitive to agents that increase cell stress and agents that damage DNA. As a result, WS cells show a drastic reduction in replicative lifespan and enter into a stage of aging prematurely. The accumulation of these damaged cells due to telomere shortening over many years can be indicative of why Werner syndrome symptoms only appear after an individual is about twenty years old.

### Sleep Disorders

In some embodiments, methods for treating a disorder comprising administering a compound described herein to a subject in need thereof wherein the disorder comprises a sleep disorder are disclosed.

A sleep disorder can be a medical disorder of the sleep patterns of a person or animal. Polysomnography and actigraphy are tests that can be ordered for diagnosis of some sleep disorders.

Sleep disorders can be classified as, for example, dyssomnias, parasomnias, circadian rhythm sleep disorders involving the timing of sleep, and other disorders including ones caused by medical or psychological conditions and sleeping sickness. Sleep disorders can include, for example, sleep apnea, narcolepsy, hypersomnia, cataplexy, and sleeping sickness. Other sleep disorders include, for example, sleepwalking, night terrors, and bed wetting.

In some embodiments, the sleep disorder includes, but is not limited to bruxism, delayed sleep phase disorder (DSPD), hypopnea syndrome, idiopathic hypersomnia, insomnia, Kleine-Levin syndrome, narcolepsy, excessive daytime sleepiness, cataplexy, night terror, nocturia, parasomnias, periodic limb movement disorder, nocturnal myoclonus, hypnic jerk, rapid eye movement sleep behavior disorder, restless leg syndrome, sleep apnea, obstructive sleep apnea, sleep paralysis, sleepwalking, somniphobia, situational circadian rhythm sleep disorder, shift worker sleep disorder, and jet lag.

### Compounds of the Disclosure.

The disclosure provides synolytic agents represented by Formulas (I) to (V) and salts thereof for the treatment of senescence-associated conditions. In certain embodiments, the disclosure provides a compound of Formula I:

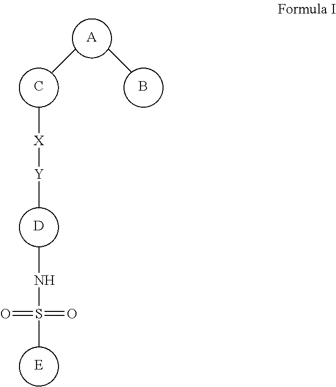

or a pharmaceutically-acceptable salt thereof, wherein: A is absent, alkylene, alkenylene, alkynylene, arylene, cycloalkylene, heteroarylene, heterocycloalkylene, aminocarbonyl, alkoxycarbonyl, an ether linkage, a sulfonyl linkage, a carbamate linkage, a carbonate linkage, an amide linkage, a urea linkage, or an ester linkage, any of which is substituted or unsubstituted: B is alkyl, alkenyl, alkynyl, aryl, cycloalkyl, heteroaryl, heterocycloalkyl, acyl, aminocarbonyl, alkoxycarbonyl, alkoxy, cycloalkyloxy, heterocycloalkyloxy, sulfonyl, carbamate, carbonate, amide, amine, urea, or ester; any of which is substituted or unsubstituted, or H; E is alkyl, alkenyl, alkynyl, aryl, cycloalkyl, heteroaryl, heterocycloalkyl, acyl, aminocarbonyl, alkoxycarbonyl, alkoxy, cycloalkyloxy, heterocycloalkyloxy, sulfonyl, carbamate, carbonate, amide, amine, urea, or ester; any of which is substituted or unsubstituted, or H; C is absent, alkylene, alkenylene, alkynylene, arylene, cycloalkylene, heteroarylene, heterocycloalkylene, aminocarbonyl, alkoxycarbonyl, an ether linkage, a sulfonyl linkage, a carbamate linkage, a carbonate linkage, an amide linkage, a urea linkage, or an ester linkage, any of which is substituted or unsubstituted; D is is absent, alkylene, alkenylene, alkynylene, arylene, cycloalkylene, heteroarylene, heterocycloalkylene, aminocarbonyl, alkoxycarbonyl, an ether linkage, a sulfonyl linkage, a carbamate linkage, a carbonate linkage, an amide linkage, a urea linkage, or an ester linkage, any of which is substituted or unsubstituted; X and Y are independently absent, O, S, CO, SO₂, SO, PO₃H, NR′, BR′, PR′, POR′, alkylene, cycloalkylene, alkenylene, cycloalkenylene, alkynylene, arylene, heteroarylene, or heterocyclylene, any of which is substituted or unsubstituted, or any combination thereof; or X and Y are members of a ring; each R′ is independently alkyl, alkenyl, alkynyl, aryl, aralkyl, cycloalkyl, heteroaryl, heterocycloalkyl, acyl, aminocarbonyl, alkoxycarbonyl, alkoxy, cycloalkyloxy, heterocycloalkyloxy, sulfonyl, cycloalkeny, or H, or a pharmaceutically-acceptable salt thereof.

Non-limiting examples of rings A, B, C, D and E include:

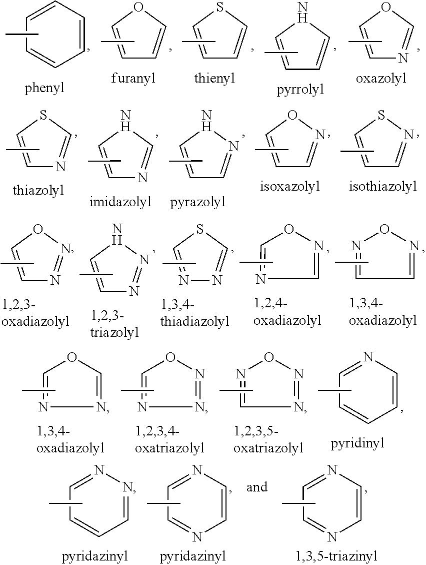

any of which is unsubstituted or substituted at any number of positions.

Non-limiting examples of ring B include:

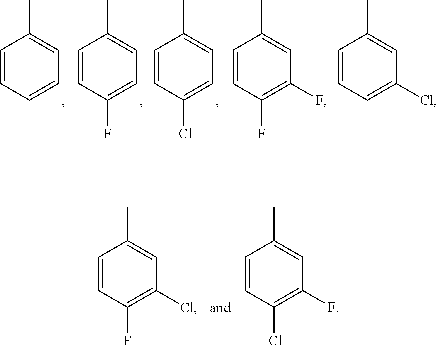

Non-limiting examples of ring A include:

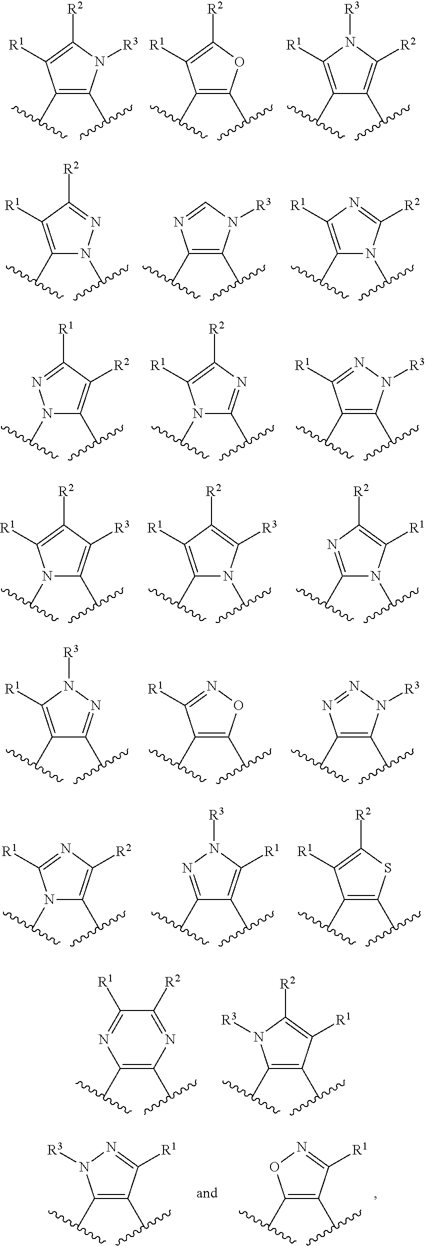

wherein: R¹ and R² are independently hydrogen, CN, NO₂, halo, optionally substituted alkyl, optionally substituted cycloalkyl, optionally substituted alkenyl, optionally substituted cycloalkenyl, optionally substituted alkynyl, optionally substituted aryl, optionally substituted heteroaryl, optionally substituted heterocycloalkyl, OR′, SR′, NR′R″, COR′, CO₂R′, OCOR′, CONR′R″, CONR′SO₂R″, NR′COR″, NR′CONR″R′″, NR′C═SNR″R′″, NR′SO₂R″, SO₂R′, or SO₂NR′R″; R³ is hydrogen, optionally substituted alkyl, optionally substituted cycloalkyl, optionally substituted alkenyl, optionally substituted cycloalkenyl, optionally substituted alkynyl, optionally substituted aryl, optionally substituted heteroaryl, optionally substituted heterocycloalkyl, OR′, NR′R″, OCOR′, COR′, CO₂R′, CONR′R″, CONR′SO₂R″, C₁₋₃alkyleneCH(OH)CH₂OH, SO₂R′ or SO₂NR′R″; R′, R″, and R′″ are independently chosen from hydrogen, optionally substituted alkyl, optionally substituted cycloalkyl, optionally substituted alkenyl, optionally substituted cycloalkenyl, optionally substituted alkynyl, optionally substituted aryl, optionally substituted heteroaryl, C₁₋₃alkyleneheterocycloalkyl and optionally substituted heterocycloalkyl; or R′ and R″, or R″ and R′″, together with the atoms to which they are bound form a ring that is substituted or unsubstituted.

Non-limiting examples of the A ring include:

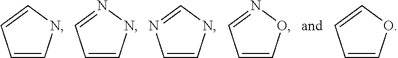

In some embodiments, a non-aromatic nitrogen atom of the A ring is substituted with C₁₋₆alkyl, for example, methyl, ethyl, n-propyl, isopropyl, or n-butyl; cycloalkyl, for example, cyclopropyl; —(CH₂)₁₋₃N(CH₃)₂, or —(CH₂)₁₋₃CH(OH)CH₂OH. In some embodiments, a non-aromatic nitrogen atom of the A ring and an adjacent carbon of the A ring are taken together to form a five- or six-membered ring that is fused to the A ring.

In some embodiments, one, two, or three carbon atoms of the A ring are substituted, independently, with CH₃, C₂H₅, C₃H₇, CF₃, NH₂, Cl, CN, CO₂H, C(═O)CH₃, C(═O)C₂H₅, C(═O)CF₃, SO₂C₂H₅, SO₂C₃H₇, SO₂CF₃, SO₂N(CH₃)₂, C(═O)NHSO₂CH₃, C(═O)NH₂, C(═O)NH₂, C(═O)NHCH₃, C(═O)NH(CH₂)₁₋₃N(CH₃)₂, C(═O)NHSO₂CH₃,

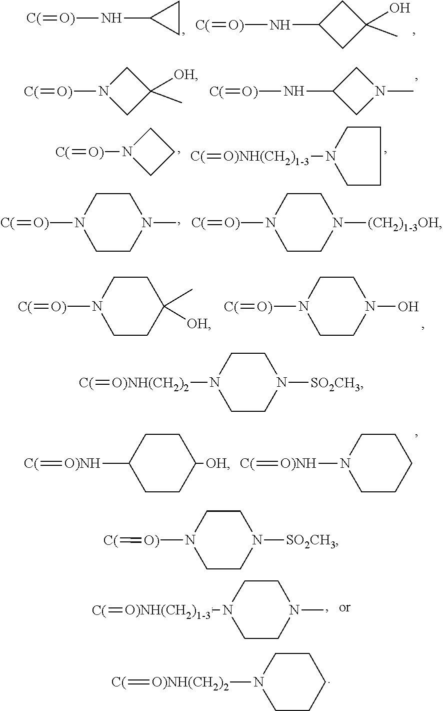

In some embodiments, the C ring is phenylene, optionally substituted with one, two, three, or four substituents, for example, fluoro, chloro, bromo, iodo, methyl, ethyl, propyl, and iso-propyl.

In some embodiments, the moiety —X—Y— is any of —CH₂CH₂—, —NHCH₂CH₂NH—, —OCH₂CH₂—O—,

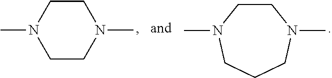

In some embodiments, the D ring is phenylene, either substituted or unsubstituted. For example, a compound herein can have the structure:

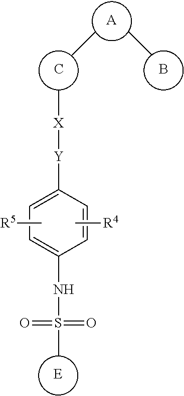

wherein R⁴ and R⁵ are independently H, CN, NO₂, CF₃, fluoro, chloro, bromo, iodo, hydroxyalkyl, alkoxy, alkyl, cycloalkyl, alkenyl, cycloalkenyl, alkynyl, aryl, heteroaryl, heterocycloalkyl, aralkyl, OR′, SR′, NR′R″, OCOR′, CO₂R′, CONR′R″, CONR′SO₂R″, NR′COR″, NR′CONR″R′″, NR′C═SNR″R′″, NR′SO₂R″, SO₂R′ or SO₂NR′R″, any of which is substituted or unsubstituted, or R⁴ and R⁵ together with the atoms to which they are bound form a ring that is fused to the phenylene, wherein the ring is substituted or unsubstituted.

In some embodiments the E ring is phenyl, having zero, one, two, three, four, or five substituents. For example, a compound herein can have the structure:

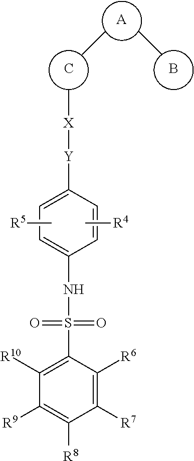

wherein R⁶, R⁷, R⁸, R⁹ and R¹⁰ are independently hydrogen, CN, NO₂, CF₃, SO₂CF₃, halo, alkyl, cycloalkyl, alkenyl, cycloalkenyl, alkynyl, aryl, aralkyl, heteroaryl, heterocycloalkyl, OR′, SR′, NR′R″, OCOR′, CO₂R′, CONR′R″, CONR′SO₂R″, NR′COR″, NR′CONR″R′″, NR′C═SNR″R′″, NR′SO₂R″, SO₂R′, SO₂NR′R″, any of which is unsubstituted or substituted.

In some embodiments, a substituent on a phenyl ring E at a position meta to the SO₂ group of Formula I is NO₂ or SO₂CF₃. In some embodiments, a substituent on a phenyl ring E at a position para to the SO₂ group of Formula I is

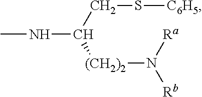

wherein R^{a} and R^{b} are independently hydrogen, methyl, optionally substituted alkyl, optionally substituted cycloalkyl, optionally substituted alkoxy, optionally substituted alkenyl, optionally substituted cycloalkenyl, optionally substituted alkynyl, optionally substituted aryl, optionally substituted heteroaryl, optionally substituted heterocycloalkyl, or

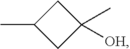

or R^{a} and R^{b} are taken together to form

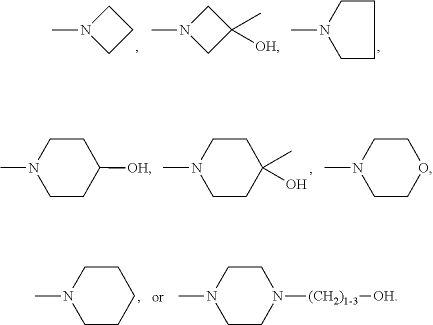

In some embodiments, compounds are of Formula II:

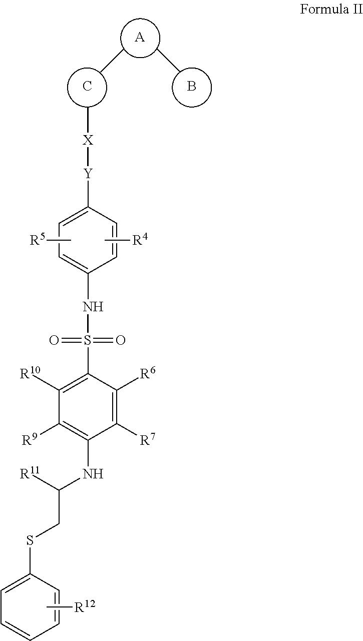

wherein the A ring is as described above; ring B is optionally substituted phenyl; ring C is optionally substituted phenylene; X, Y, R₁, R₂, R₃, R⁴, R⁵, R⁶, R⁷, R⁸, R⁹, R¹⁰, R′, R″, R′″ are defined as above; and R¹¹ and R¹² are independently hydrogen, CN, NO₂, halo, optionally substituted alkyl, optionally substituted cycloalkyl, optionally substituted alkenyl, optionally substituted cycloalkenyl, optionally substituted alkynyl, optionally substituted aryl, optionally substituted heteroaryl, optionally substituted heterocycloalkyl, OR′, SR′, NR′R″, OCOR′, CO₂R′, CONR′R″, CONR′SO₂R″, NR′COR″, NR′CONR″R′″, NR′SO₂R″, SO₂R′, or SO₂NR′R″; or a pharmaceutically acceptable salt, hydrate or solvate thereof.

In some embodiments, a compound herein is of Formula III, IV or V:

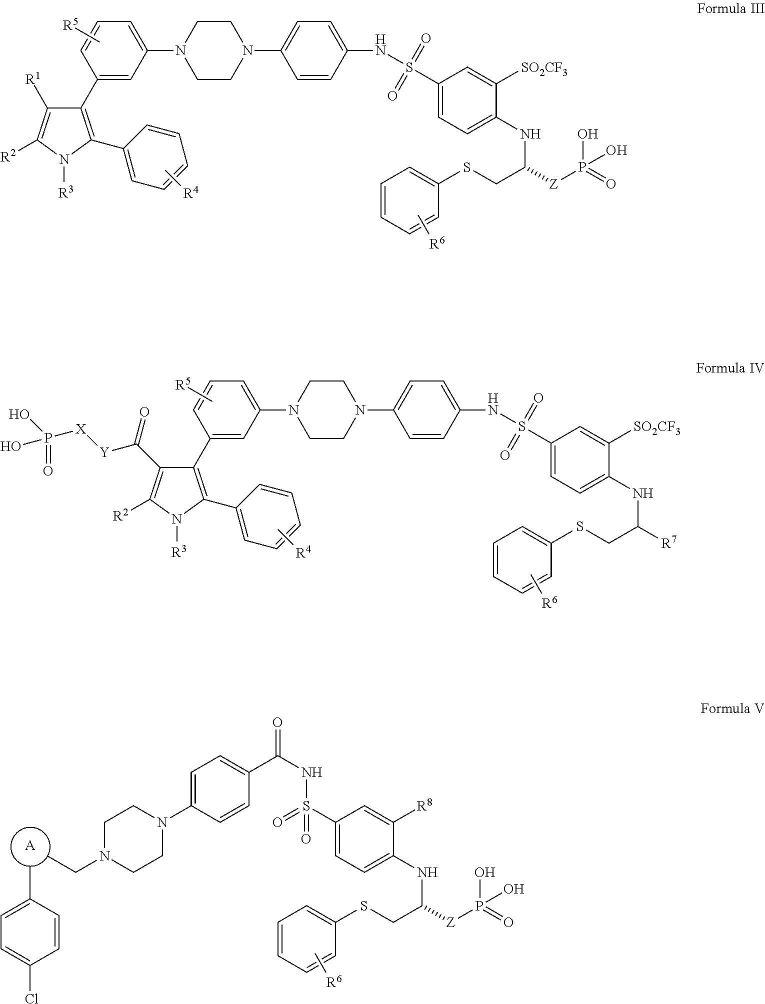

wherein Z is (CH₂)ₙ—N(R^{a})₂ or

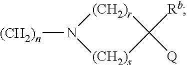

wherein:
Q is OH, alkoxy, O(CH₂)₁₋₃, NHR^{c}, NR^{c}(C₁₋₃alkylene), NR^{c}(alkylene), OC(═O)(C₁₋₃alkylene), OC(═O)(alkylene), C(═O)O(alkylene), C(═O)O(C₁₋₃alkylene), NHC(═O)(alkylene), NHC(═O)(C₁₋₃alkylene), C(═O)NHR^{c}, C(═O)NH(alkylene), C(═O)NH(C₁₋₃alkylene); R^{c} is hydrogen, CN, NO₂, CF₃, halo, hydroxyalkyl, optionally substituted alkoxy, optionally substituted alkyl, optionally substituted cycloalkyl, optionally substituted alkenyl, optionally substituted cycloalkenyl, optionally substituted alkynyl, optionally substituted aryl, optionally substituted heteroaryl, or optionally substituted heterocycloalkyl; n, r, and s are independently 1, 2, 3, 4, 5, or 6; and X, Z, the A ring, R₁, R₂, R₃, R⁴, R⁵, R⁶, R⁷, R⁸, R′, R″, R′″, R^{a} and R^{b} are defined as above.

Compounds herein include all stereoisomers, enantiomers, diastereomers, mixtures, racemates, and tautomers thereof.

Illustrative compounds of the disclosure include compounds of the following formula:

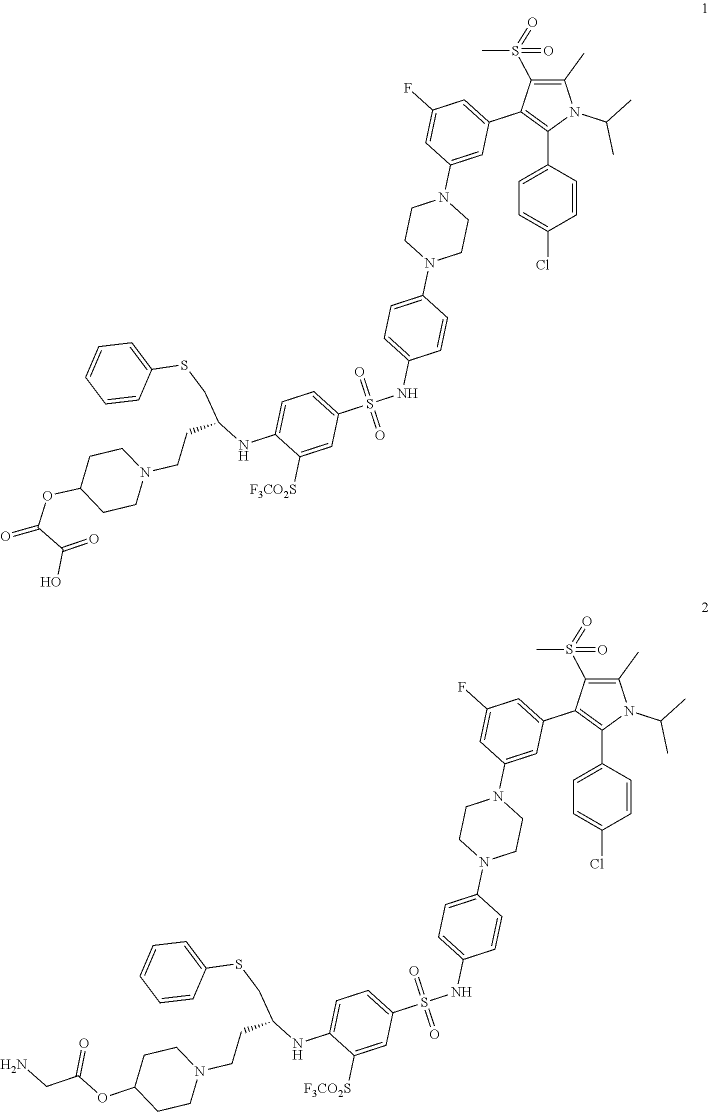

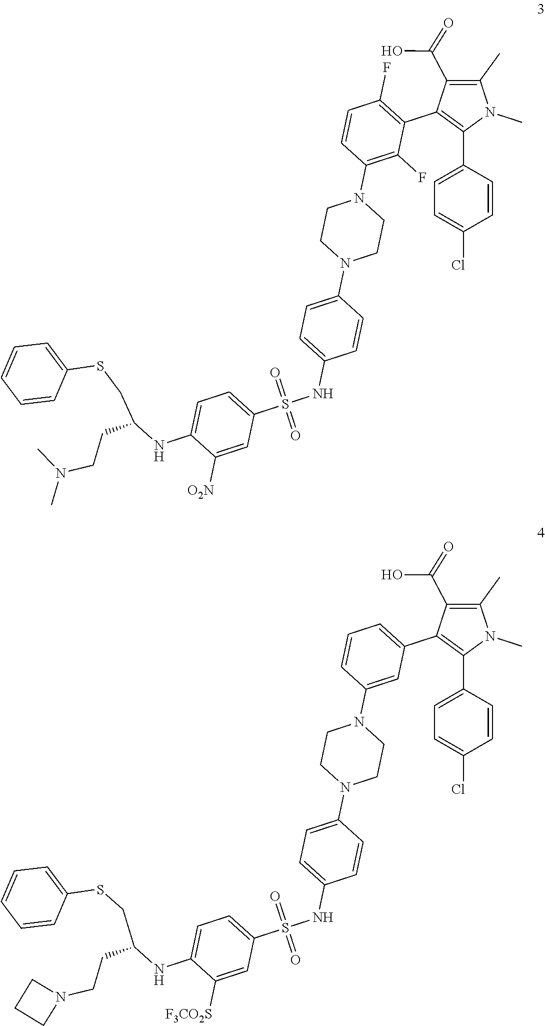

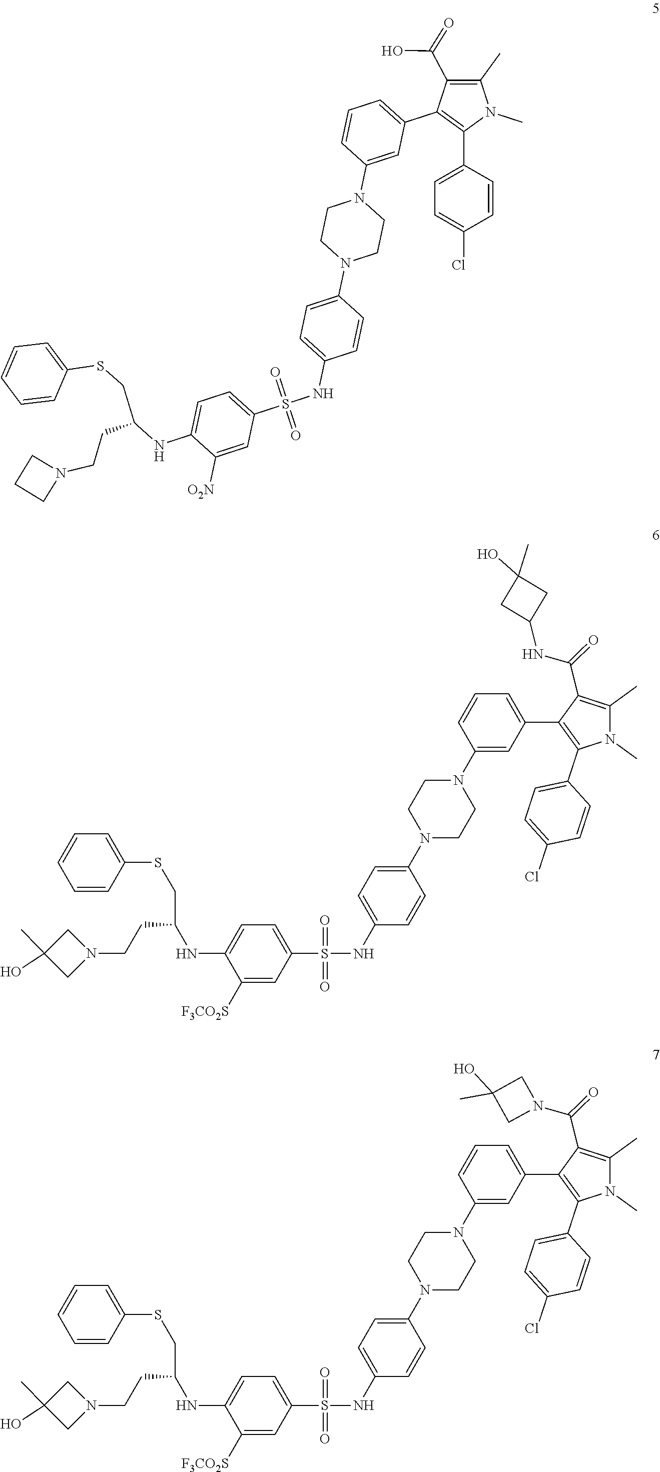

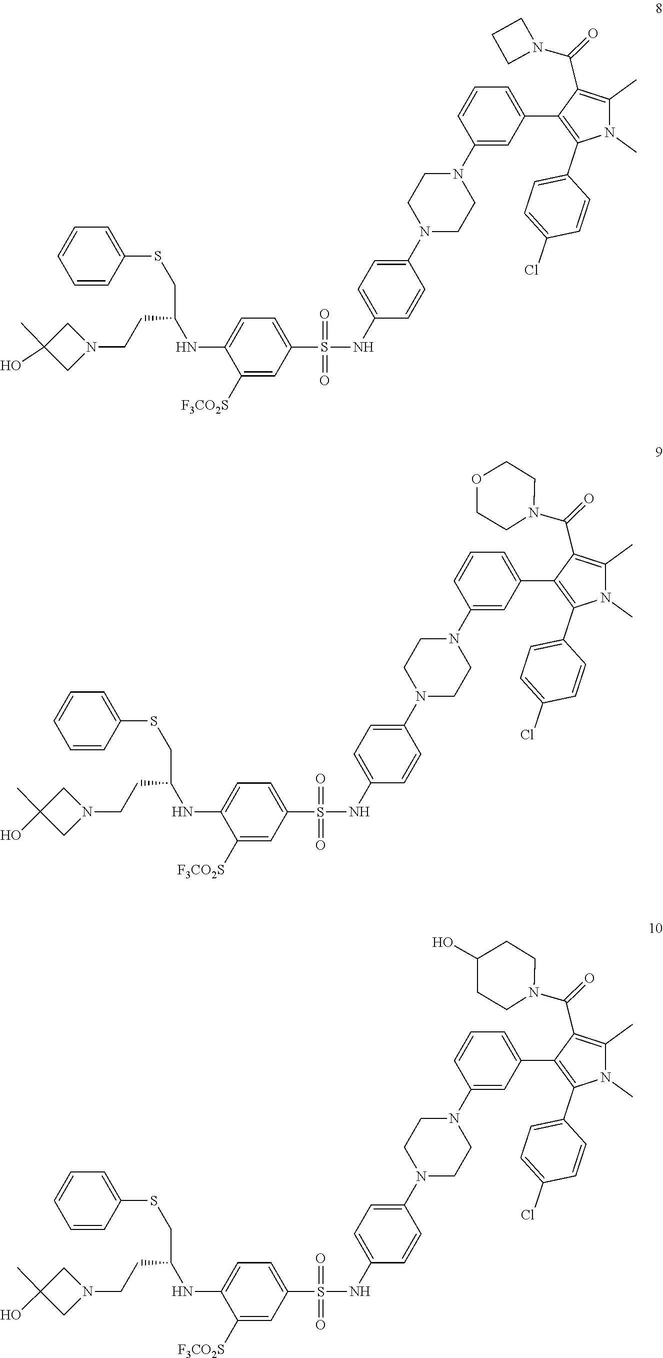

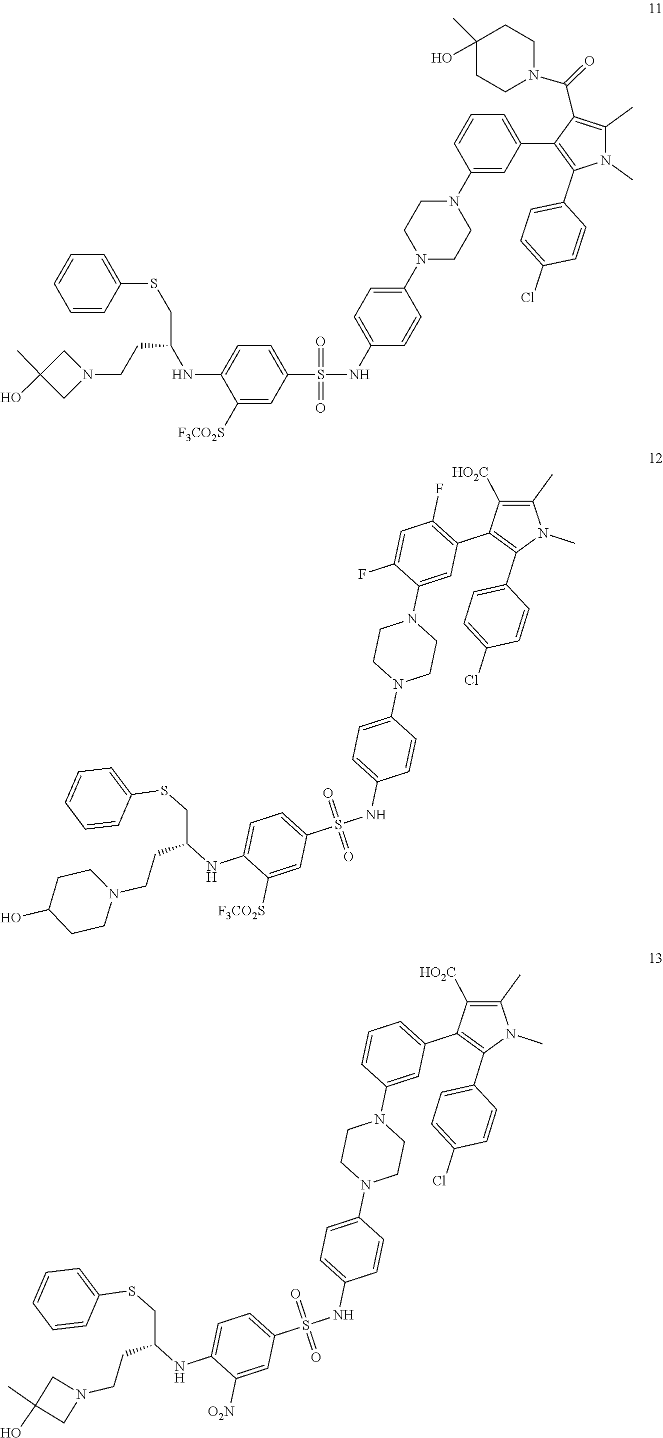

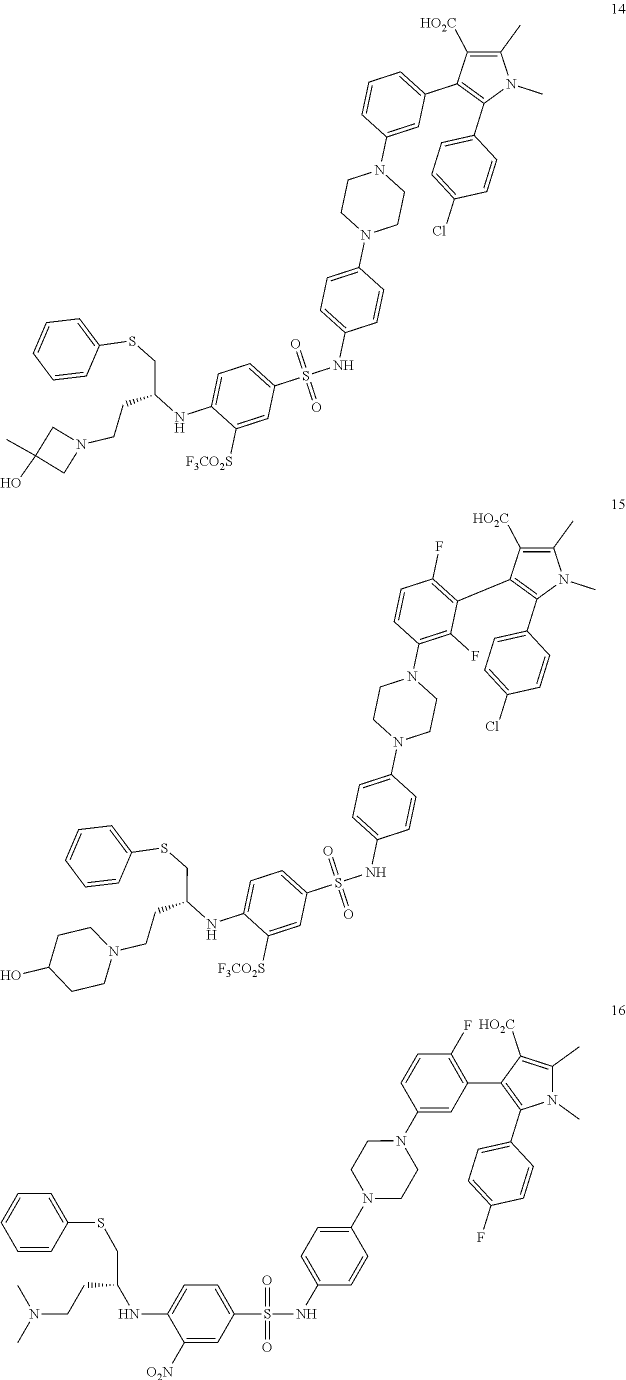

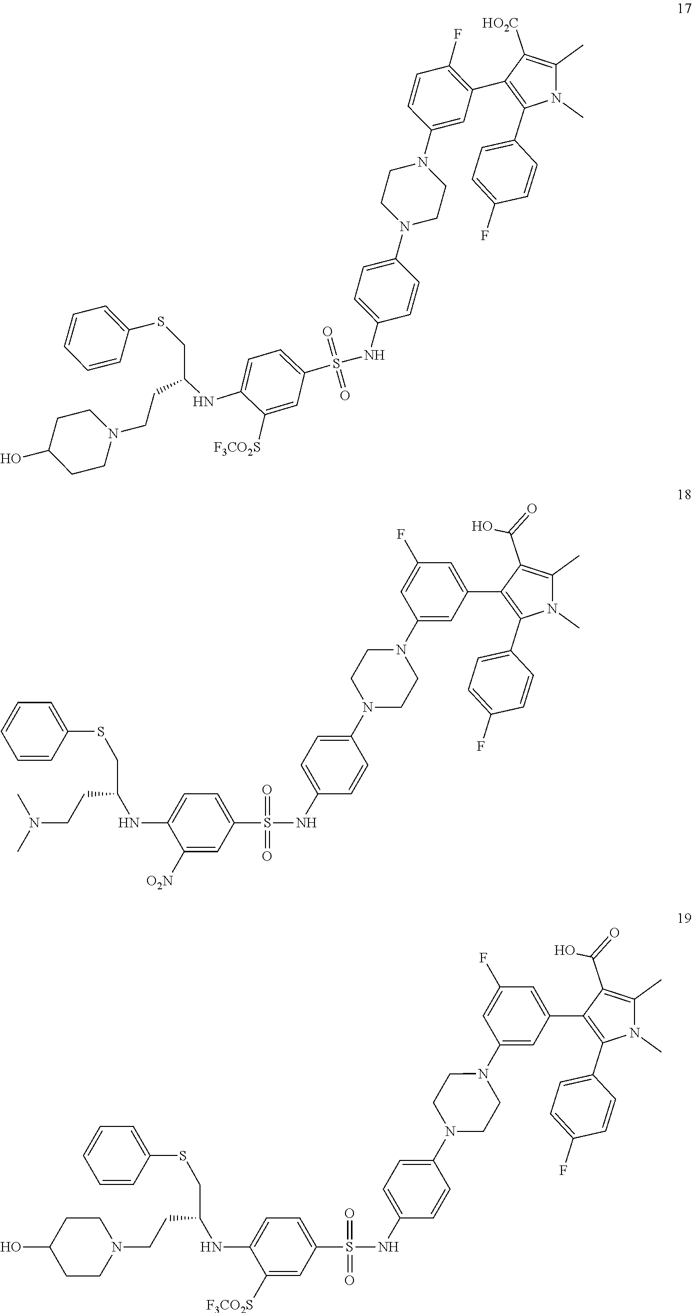

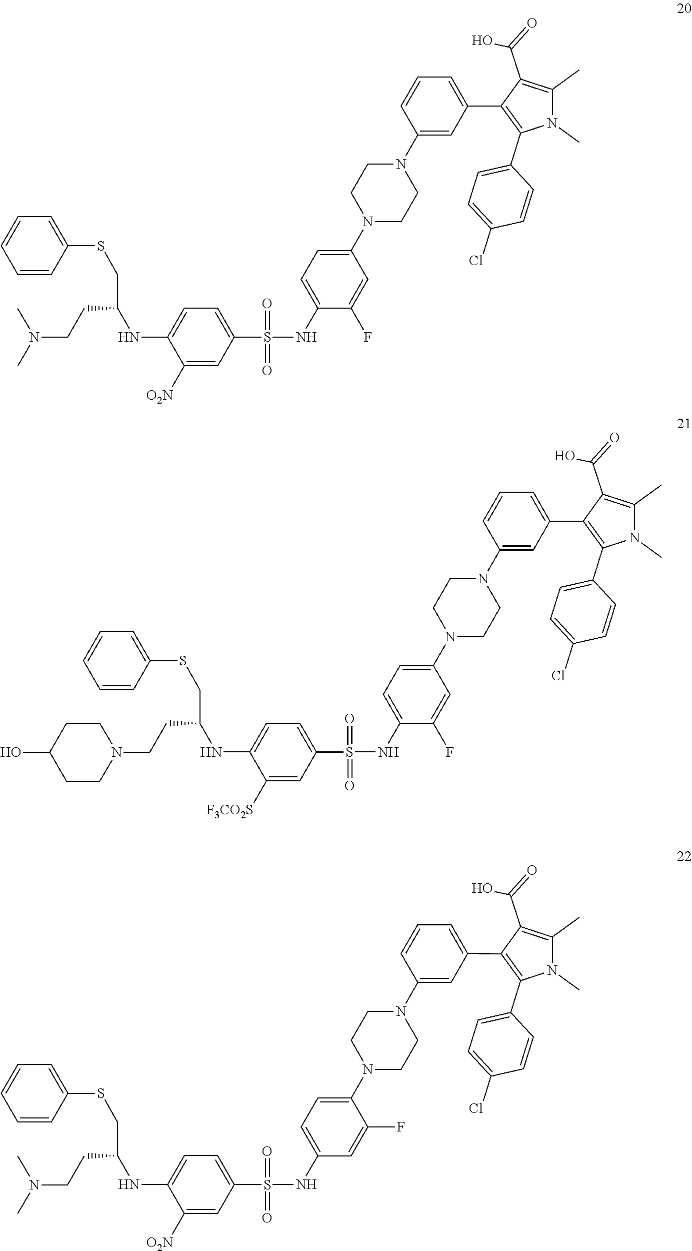

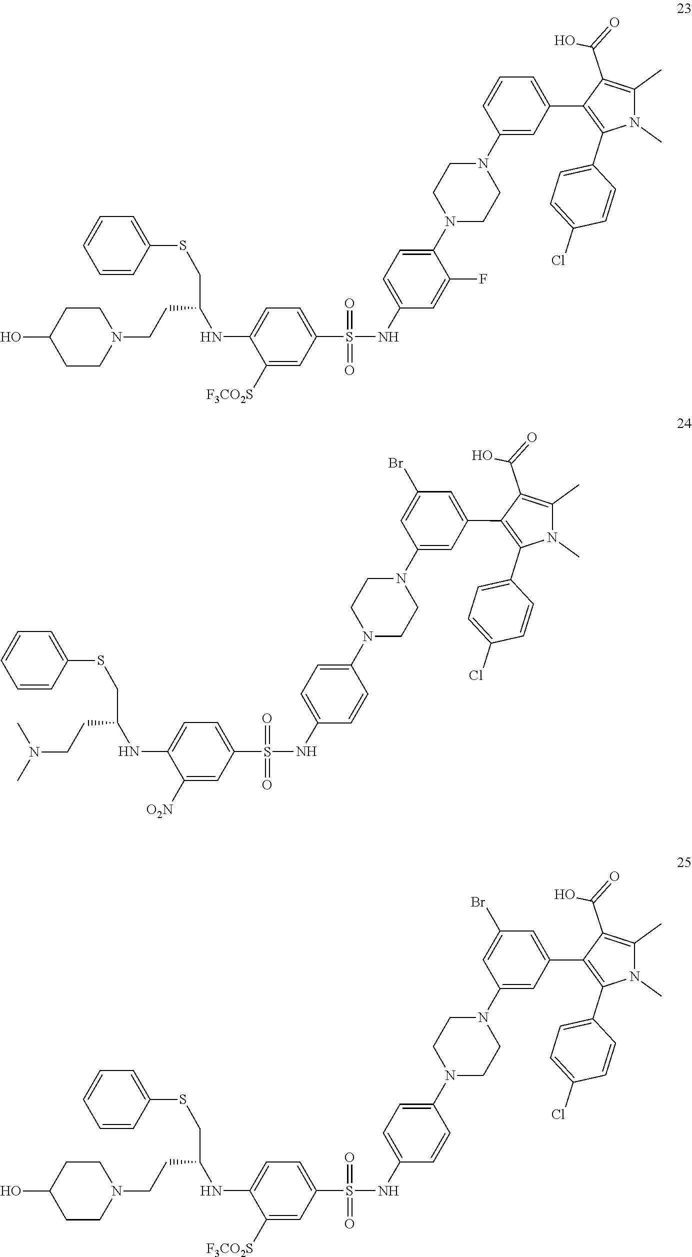

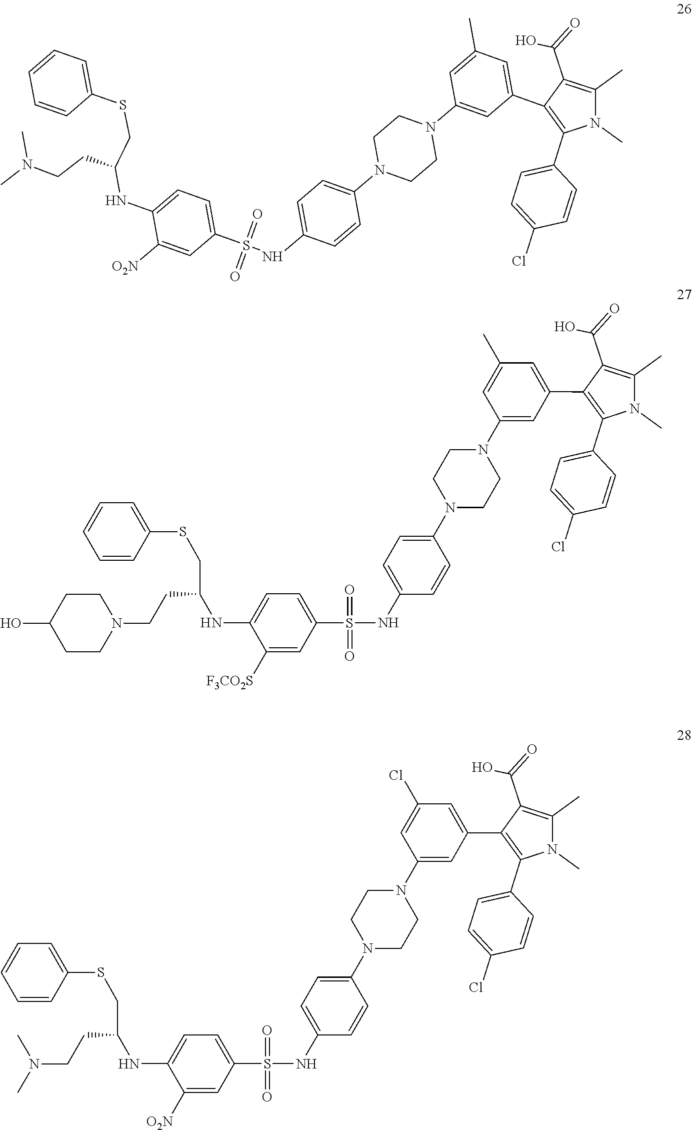

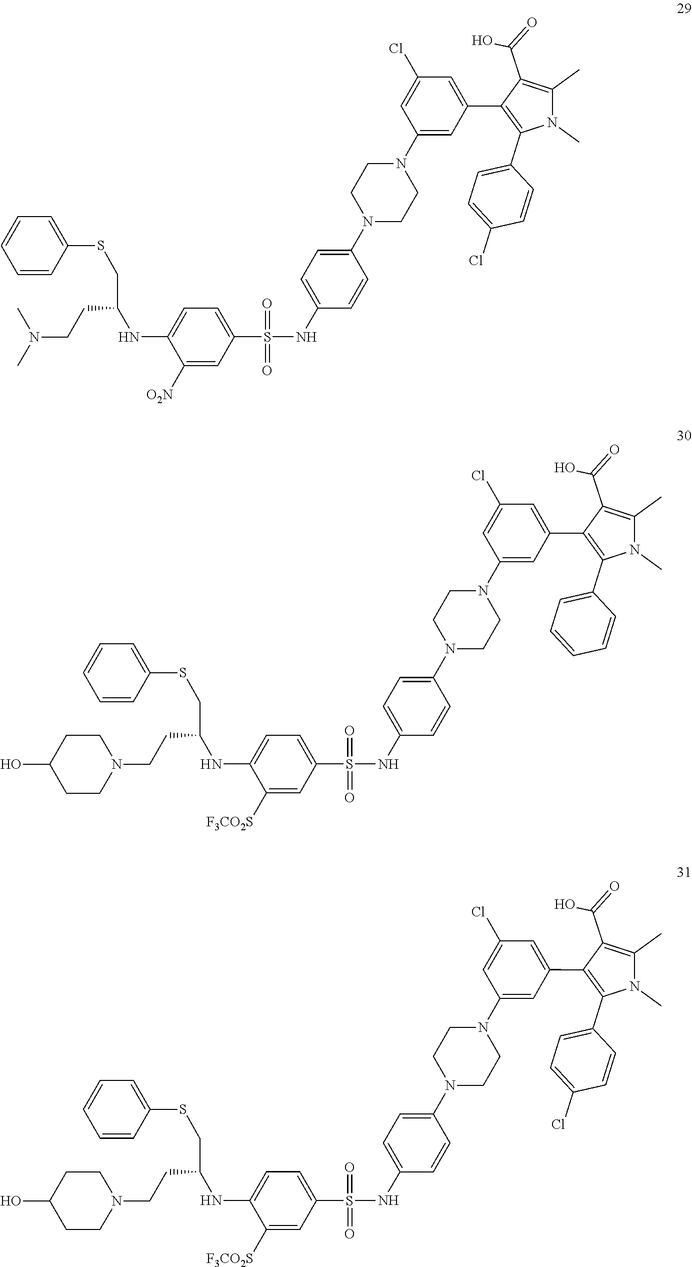

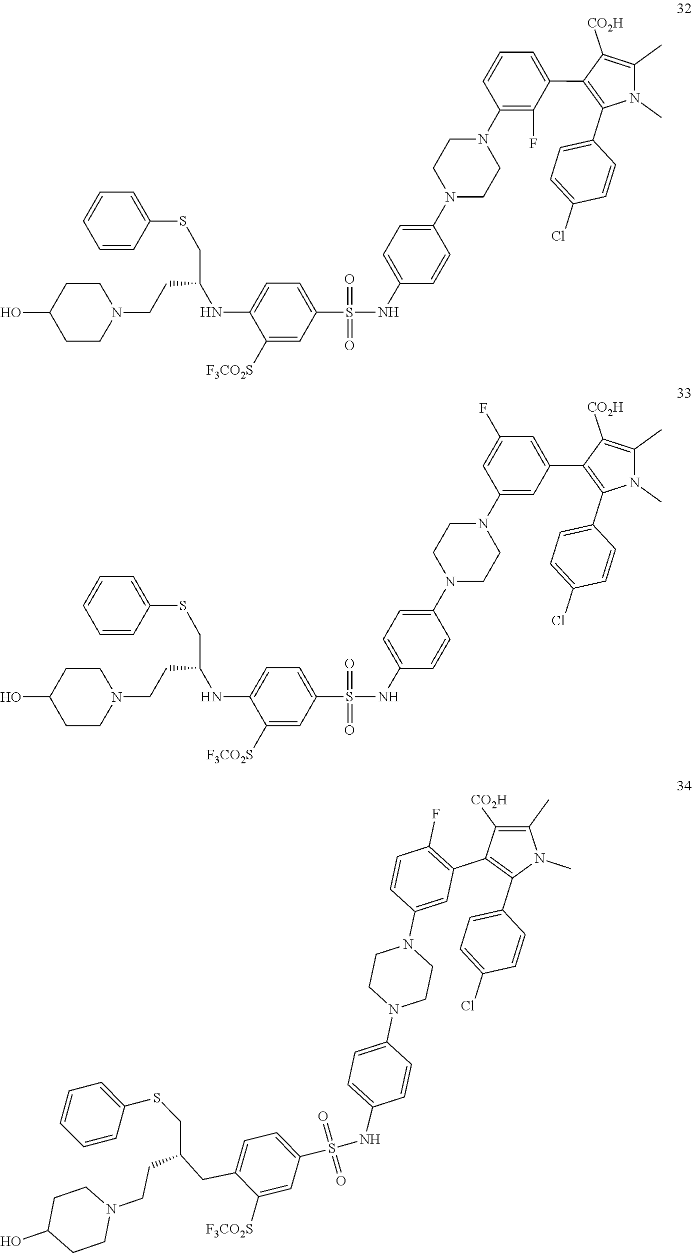

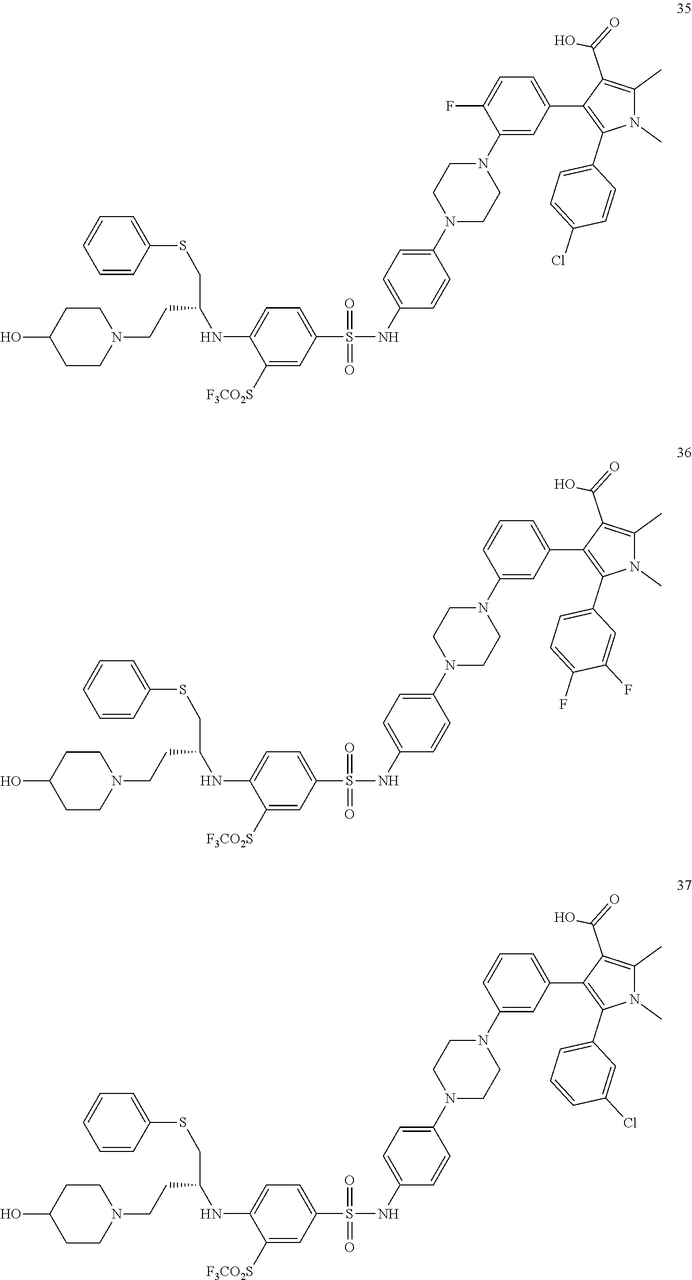

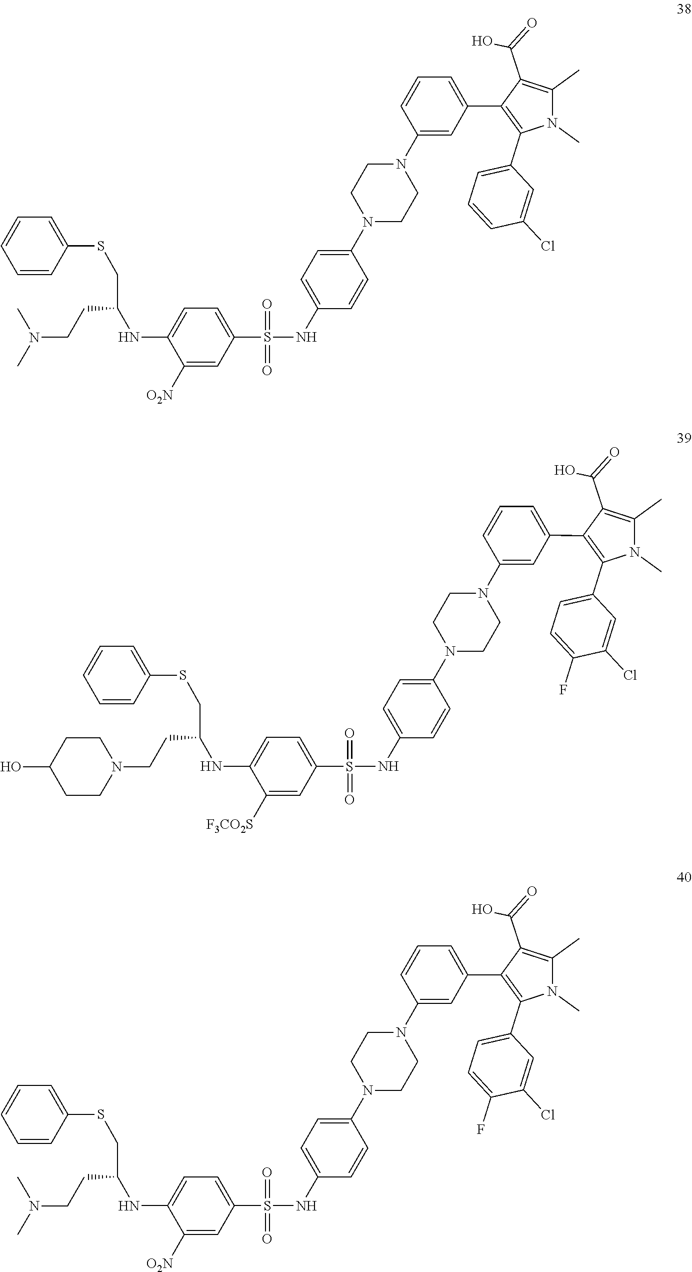

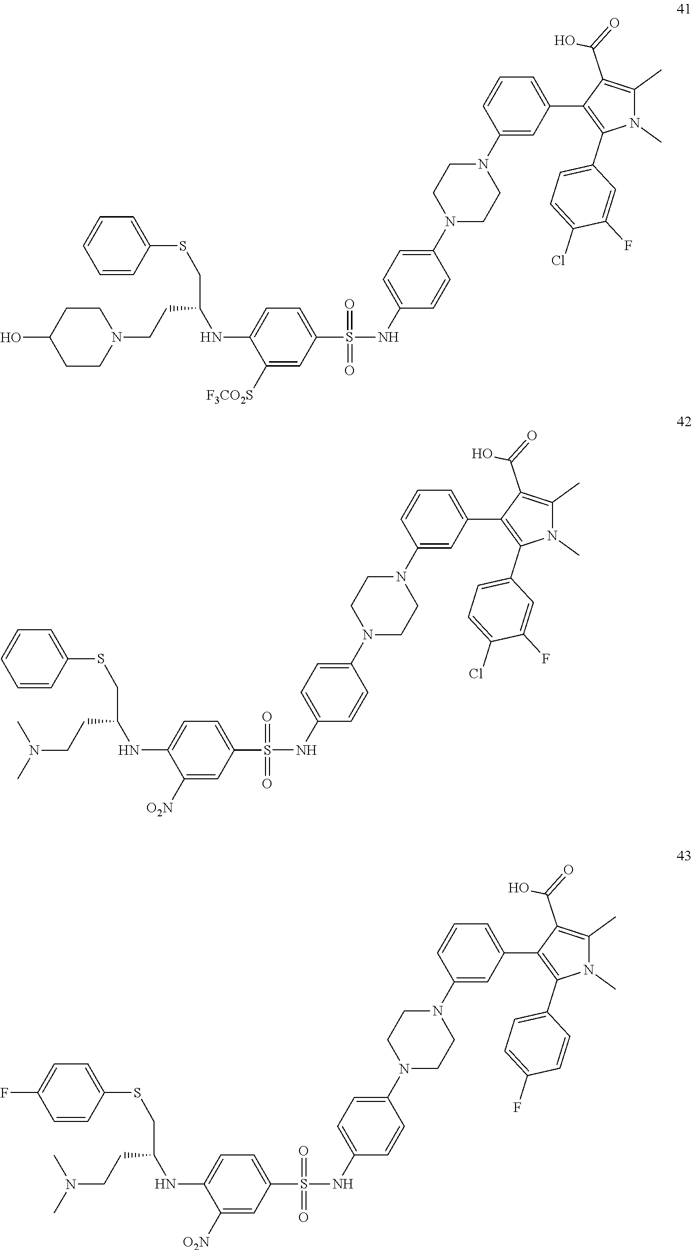

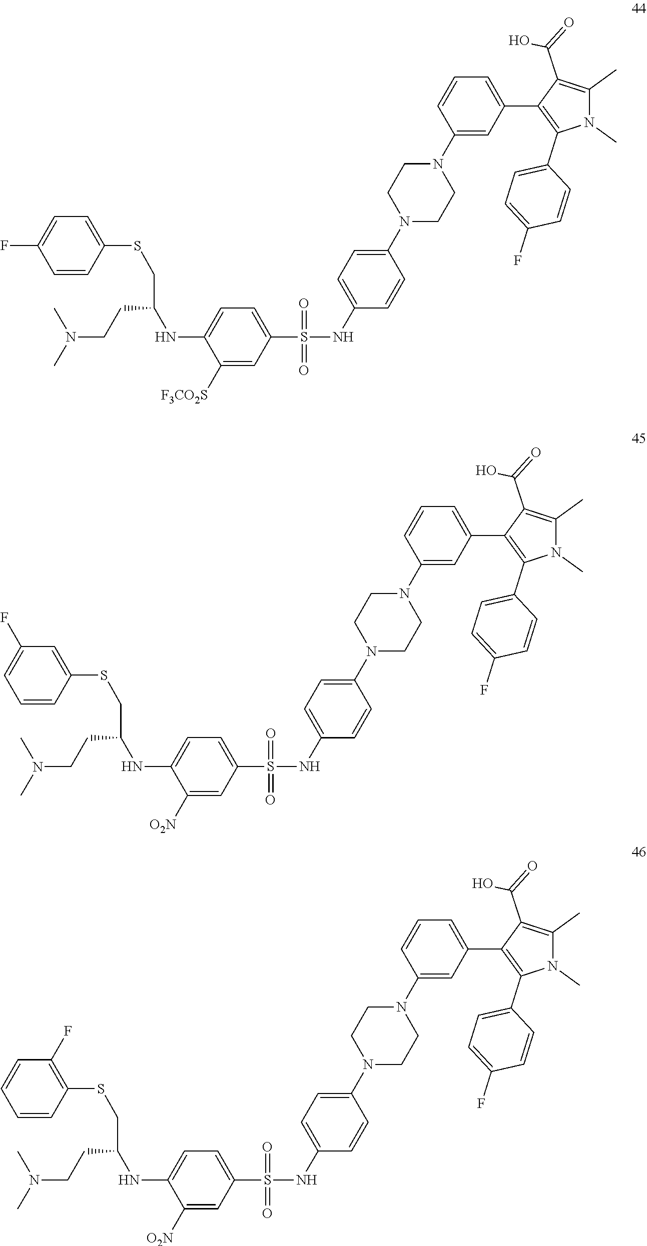

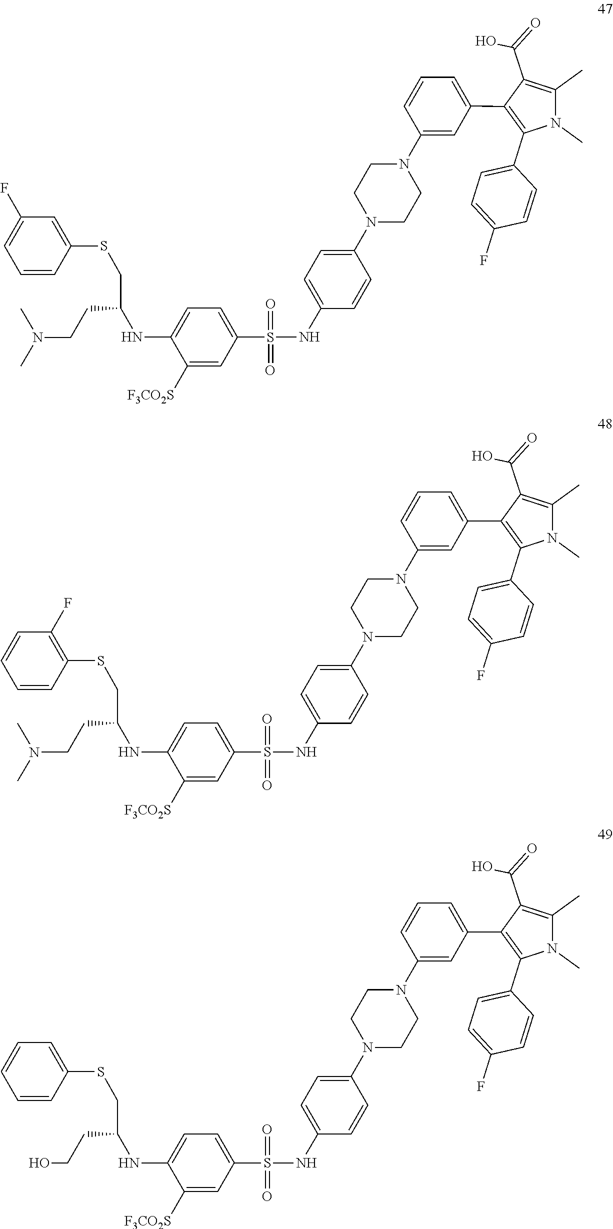

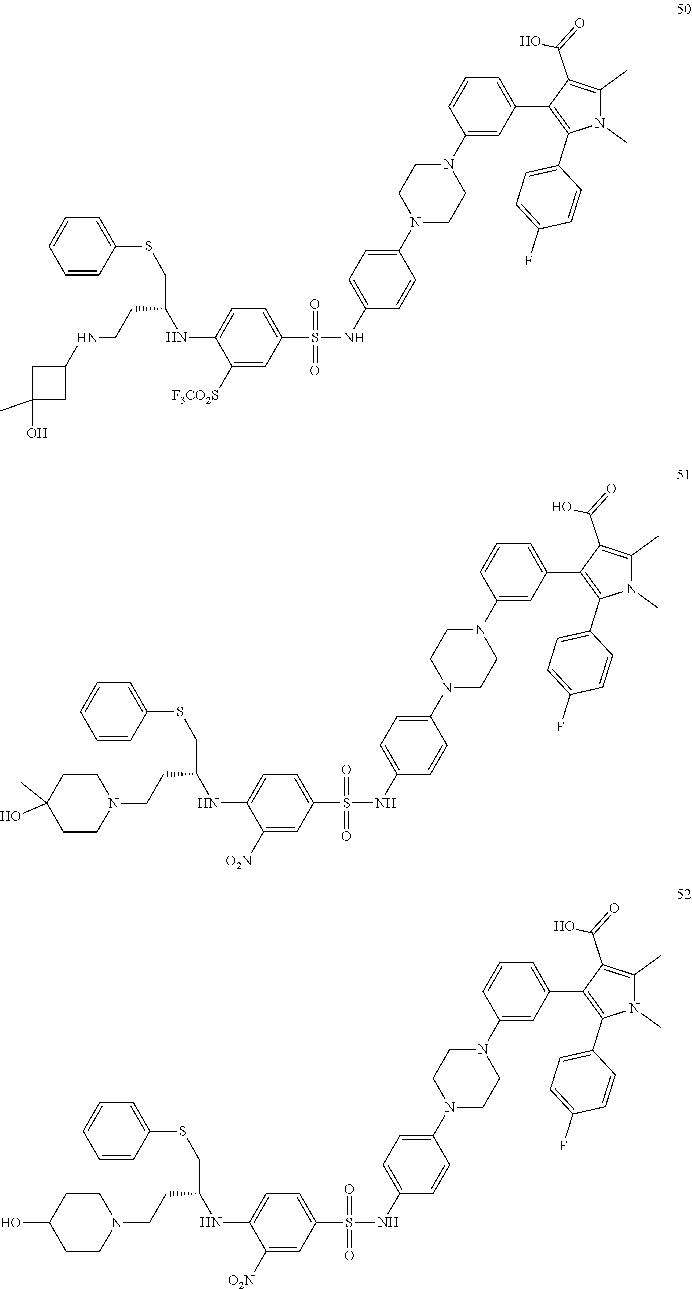

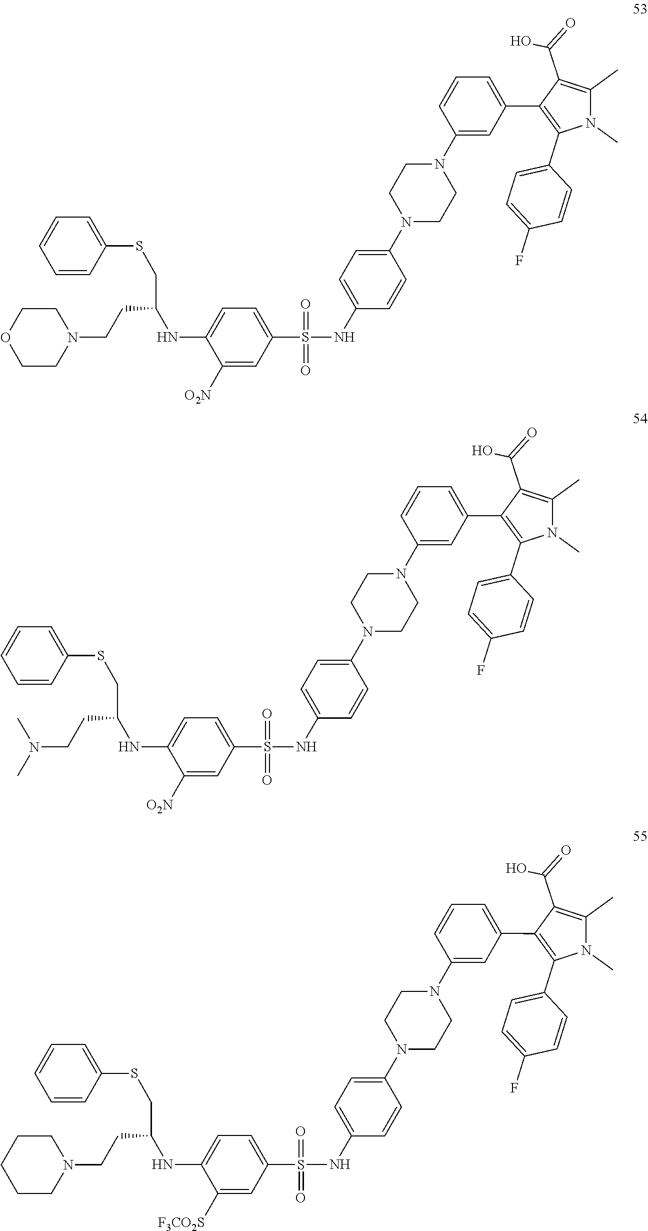

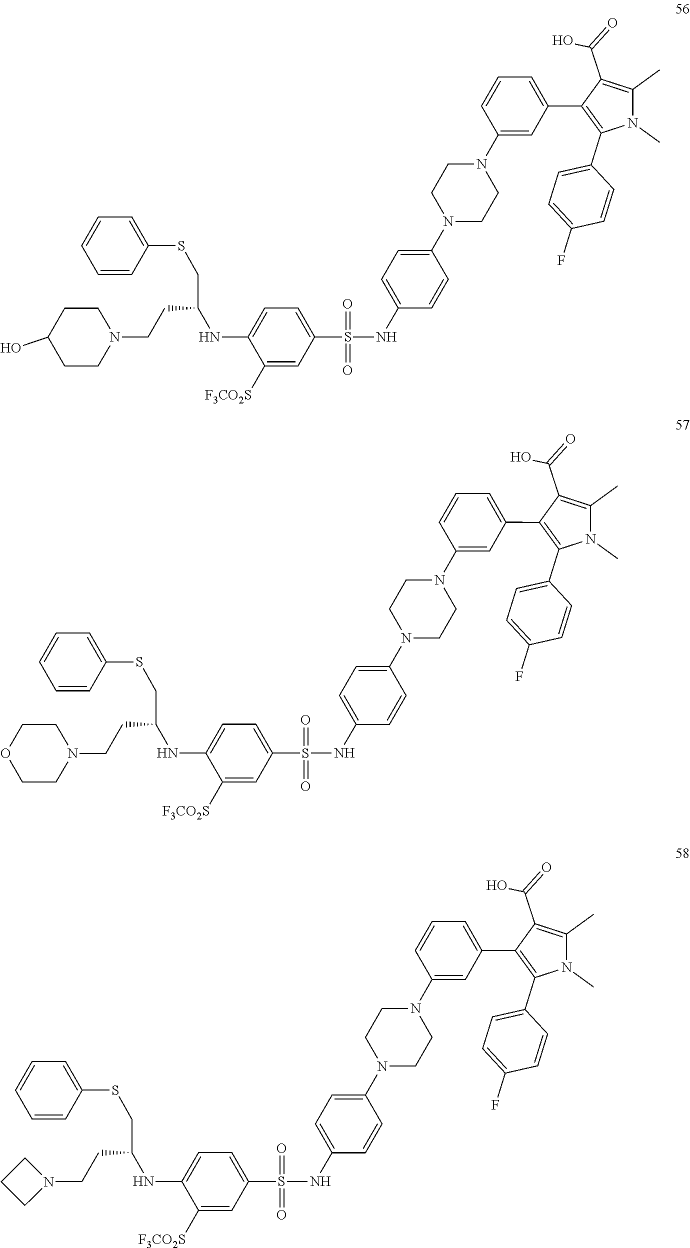

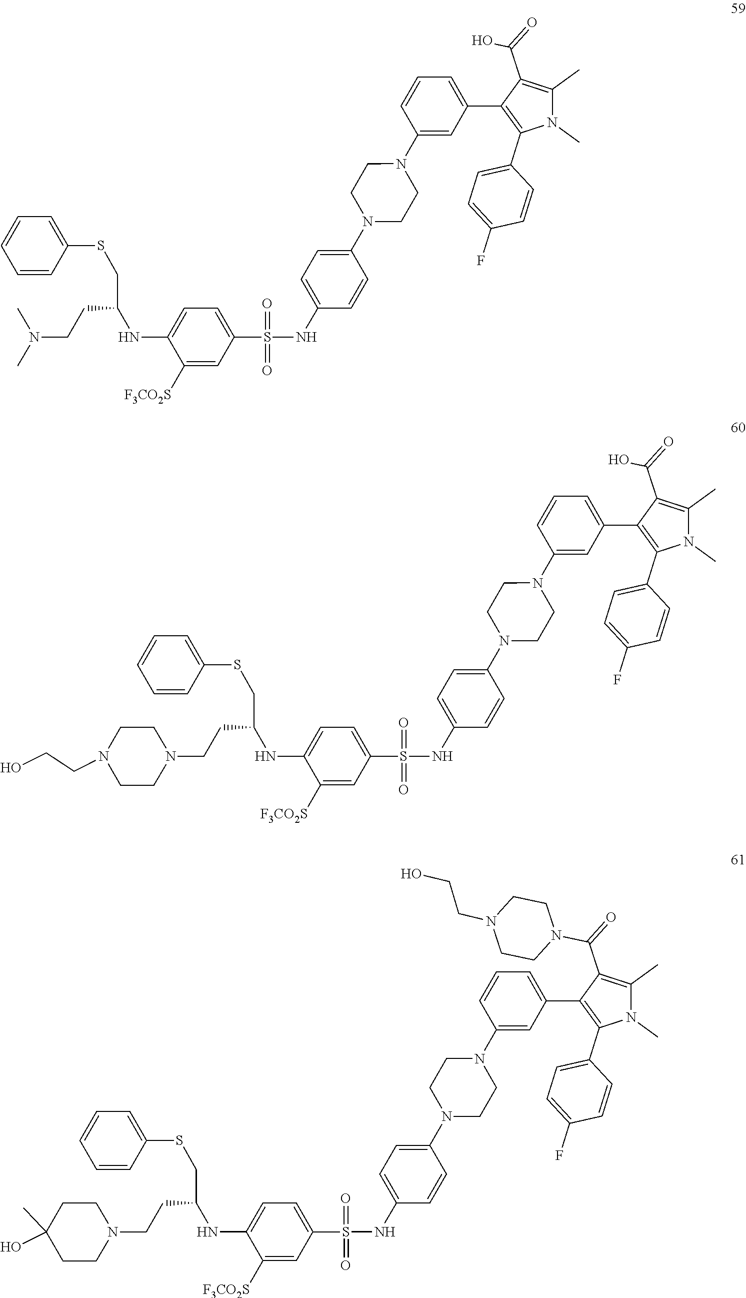

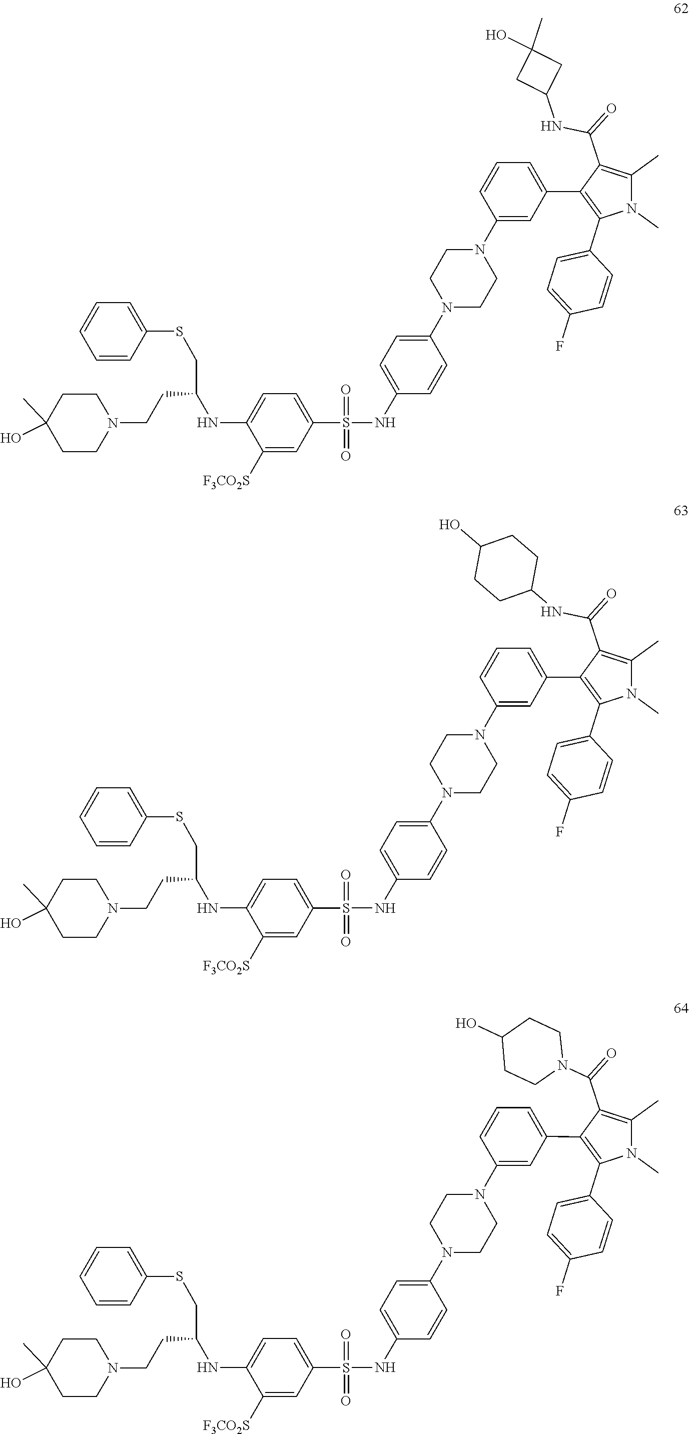

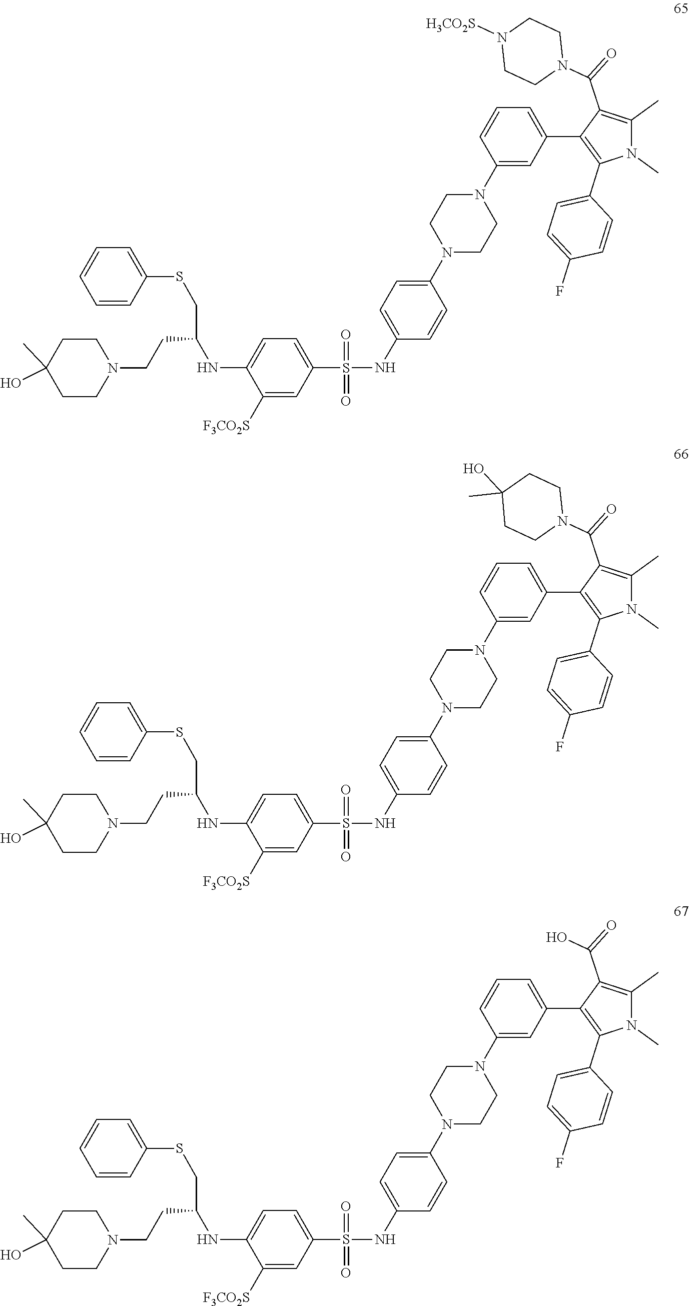

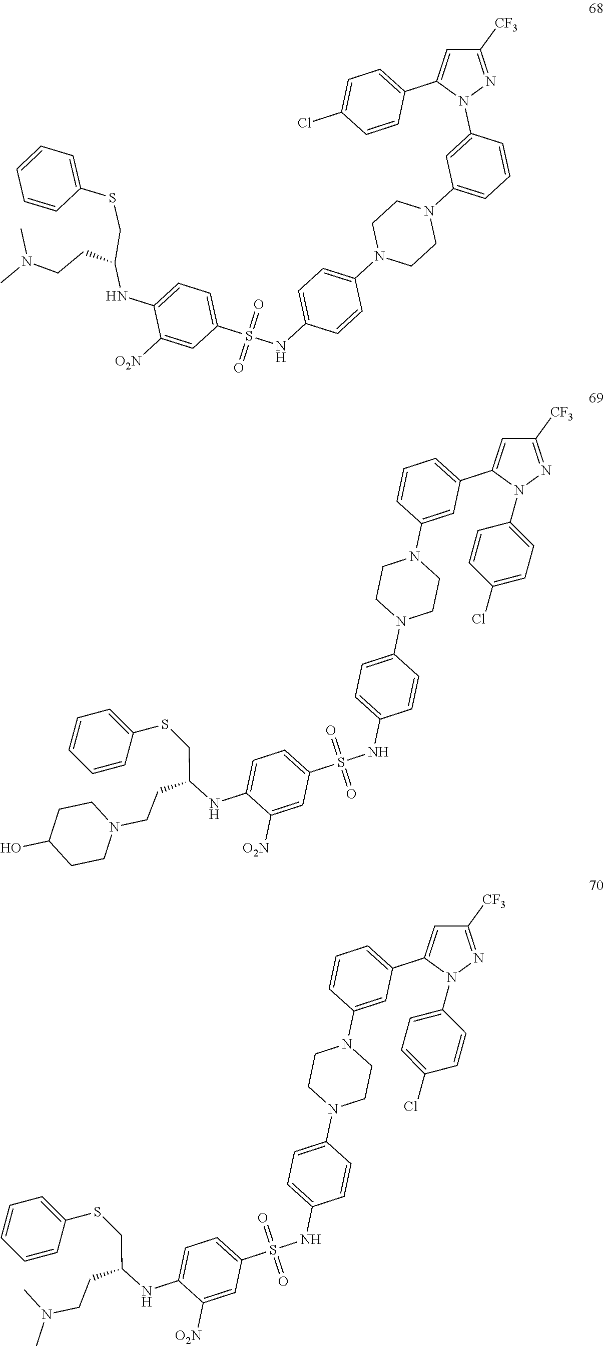 [SMILES: CN(C)CC[C@H](CSc1ccccc1)Nc1ccc(S(=O)(=O)Nc2ccc(N3CCN(c4cccc(-n5nc(C(F)(F)F)cc5-c5ccc(Cl)cc5)c4)CC3)cc2)cc1[N+](=O)[O-] ; O=[N+]([O-])c1cc(S(=O)(=O)Nc2ccc(N3CCN(c4cccc(-c5cc(C(F)(F)F)nn5-c5ccc(Cl)cc5)c4)CC3)cc2)ccc1N[C@H](CCN1CCC(O)CC1)CSc1ccccc1 ; CN(C)CC[C@H](CSc1ccccc1)Nc1ccc(S(=O)(=O)Nc2ccc(N3CCN(c4cccc(-c5cc(C(F)(F)F)nn5-c5ccc(Cl)cc5)c4)CC3)cc2)cc1[N+](=O)[O-]]

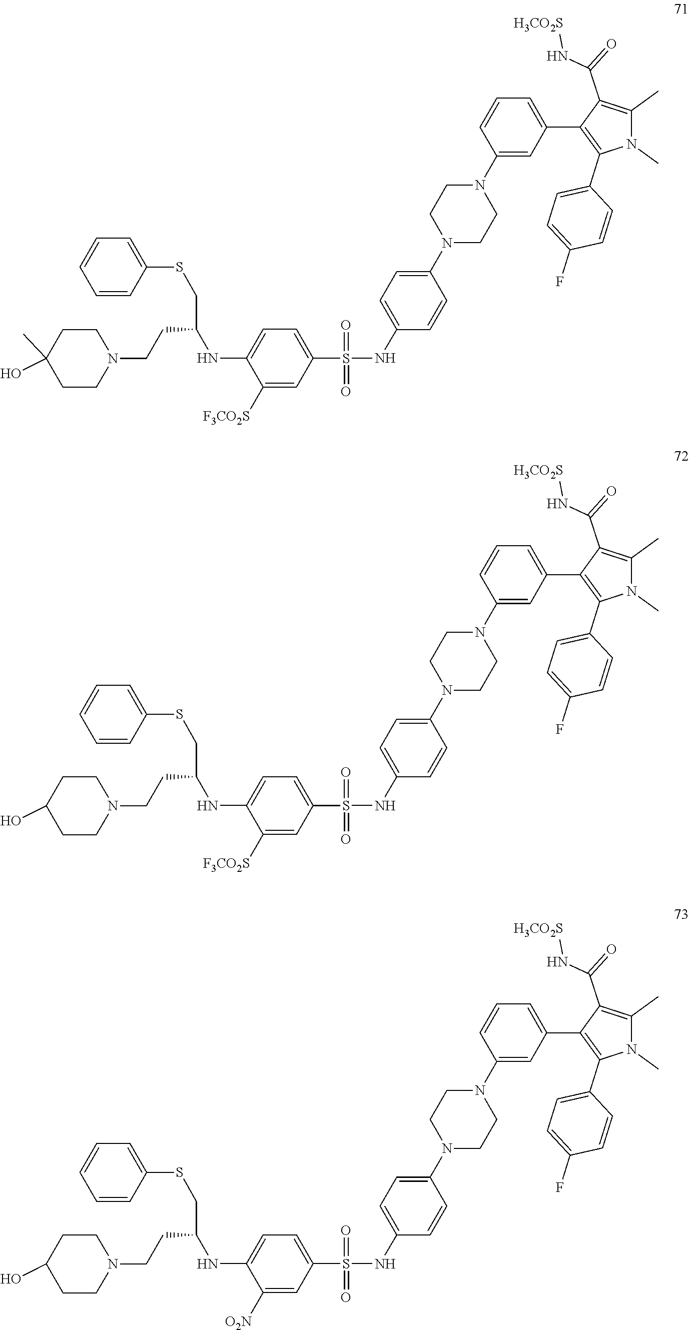

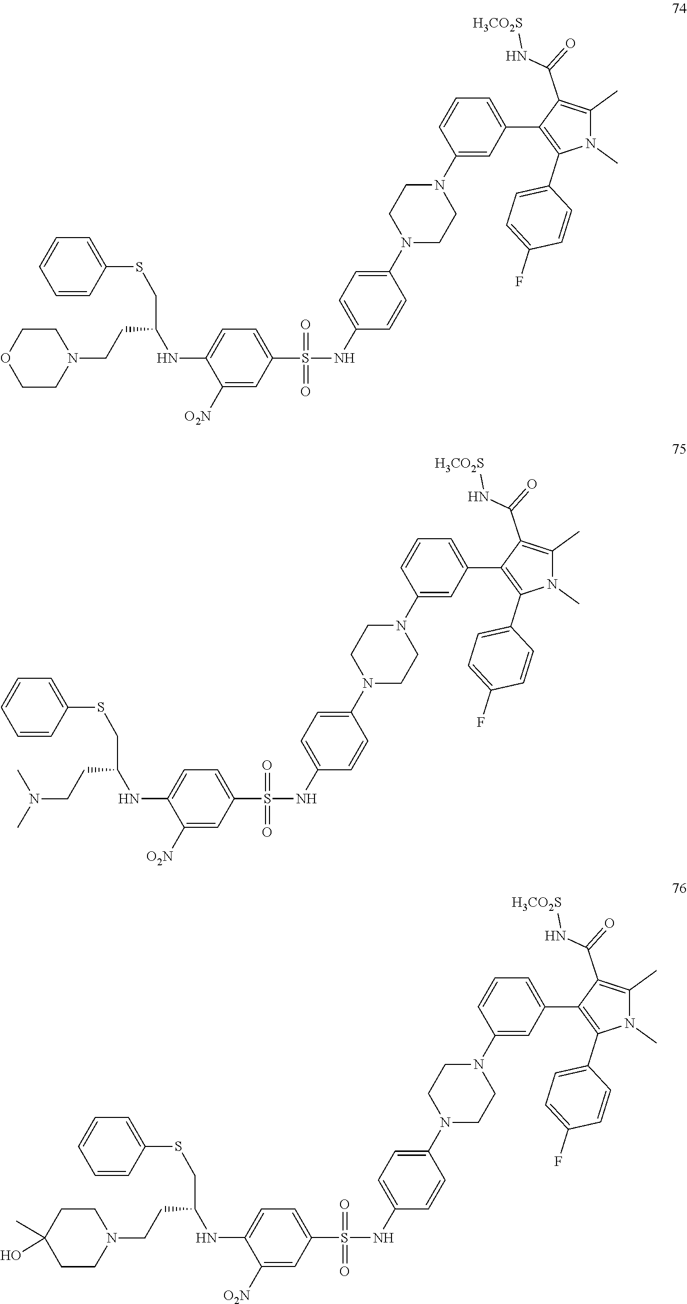

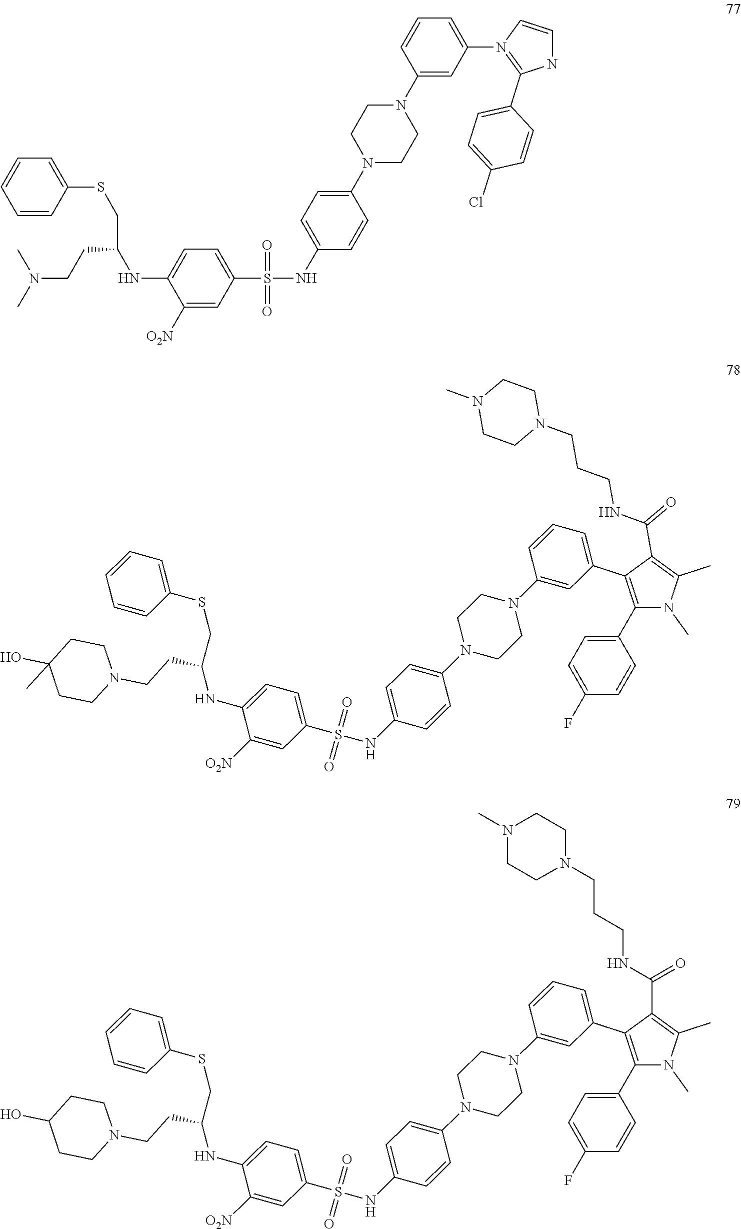

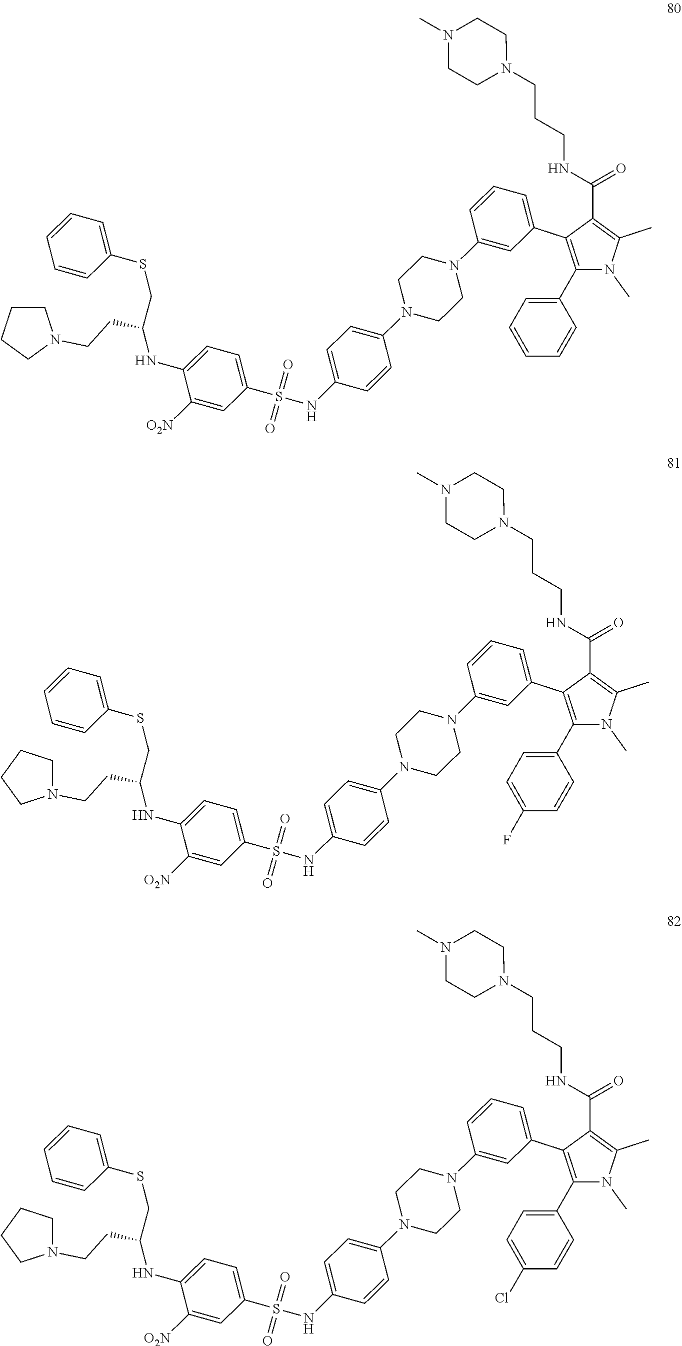 [SMILES: Cc1c(C(=O)NCCCN2CCN(C)CC2)c(-c2cccc(N3CCN(c4ccc(NS(=O)(=O)c5ccc(N[C@H](CCN6CCCC6)CSc6ccccc6)c([N+](=O)[O-])c5)cc4)CC3)c2)c(-c2ccccc2)n1C ; Cc1c(C(=O)NCCCN2CCN(C)CC2)c(-c2cccc(N3CCN(c4ccc(NS(=O)(=O)c5ccc(N[C@H](CCN6CCCC6)CSc6ccccc6)c([N+](=O)[O-])c5)cc4)CC3)c2)c(-c2ccc(F)cc2)n1C ; Cc1c(C(=O)NCCCN2CCN(C)CC2)c(-c2cccc(N3CCN(c4ccc(NS(=O)(=O)c5ccc(N[C@H](CCN6CCCC6)CSc6ccccc6)c([N+](=O)[O-])c5)cc4)CC3)c2)c(-c2ccc(Cl)cc2)n1C]

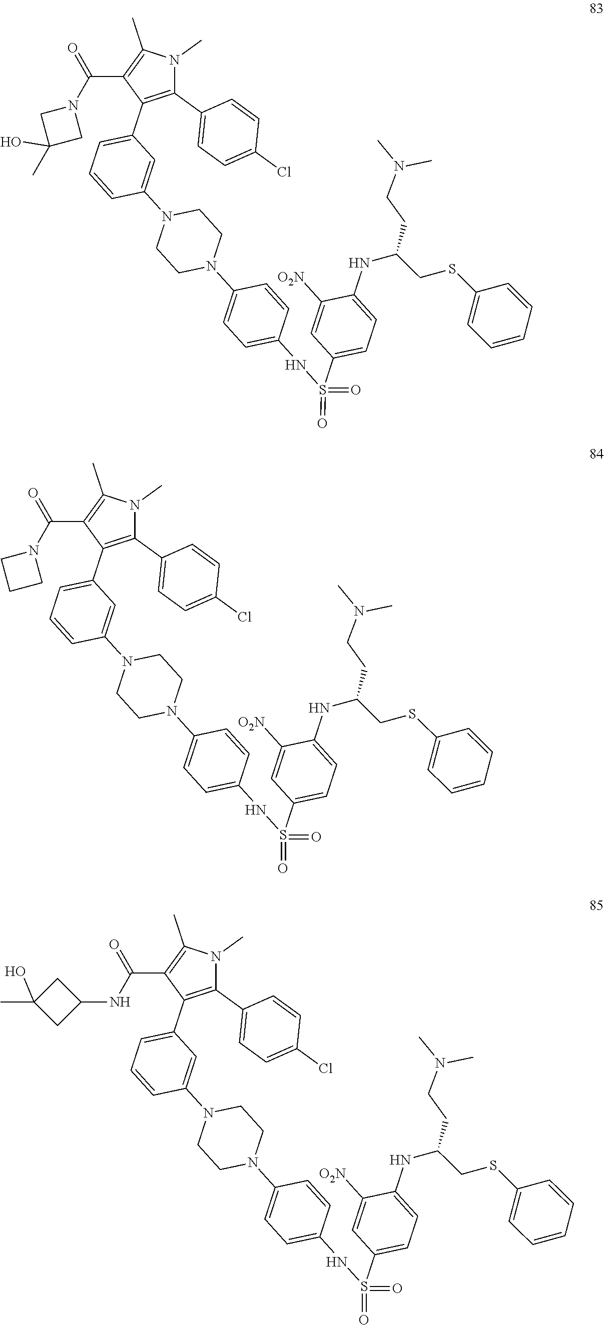 [SMILES: Cc1c(C(=O)N2CC(C)(O)C2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C ; Cc1c(C(=O)N2CCC2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C ; Cc1c(C(=O)NC2CC(C)(O)C2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C]

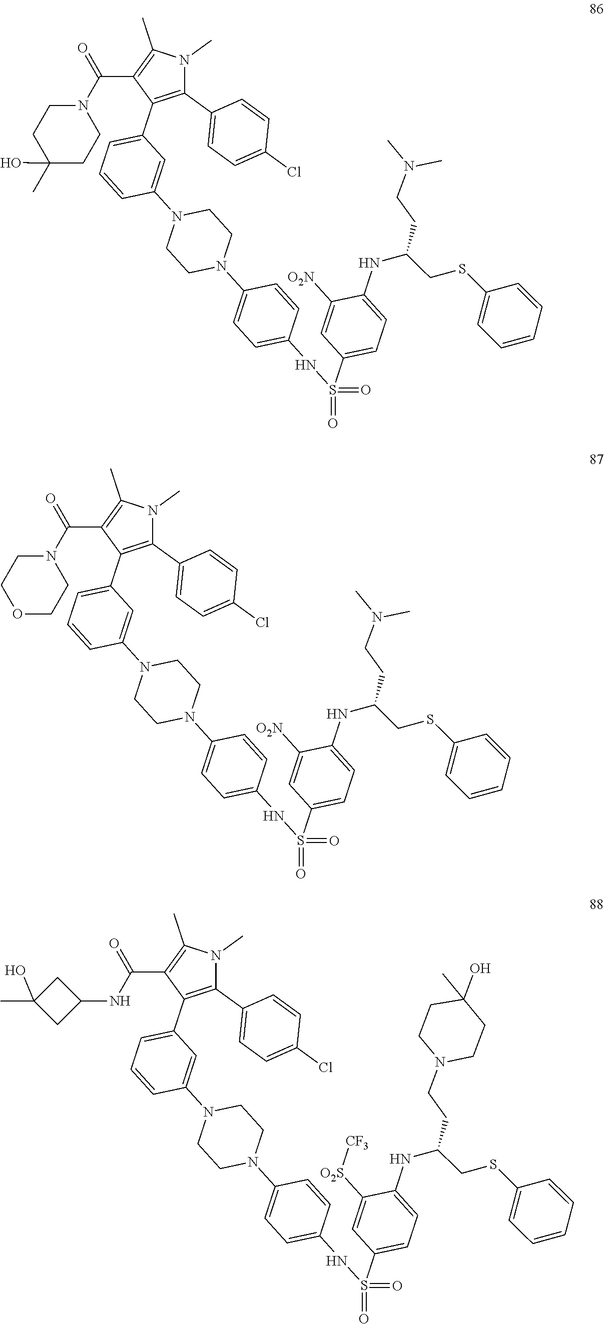 [SMILES: Cc1c(C(=O)N2CCC(C)(O)CC2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C ; Cc1c(C(=O)N2CCOCC2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C ; Cc1c(C(=O)NC2CC(C)(O)C2)c(-c2cccc(N3CCN(c4ccc(NS(=O)(=O)c5ccc(N[C@H](CCN6CCC(C)(O)CC6)CSc6ccccc6)c(S(=O)(=O)C(F)(F)F)c5)cc4)CC3)c2)c(-c2ccc(Cl)cc2)n1C]

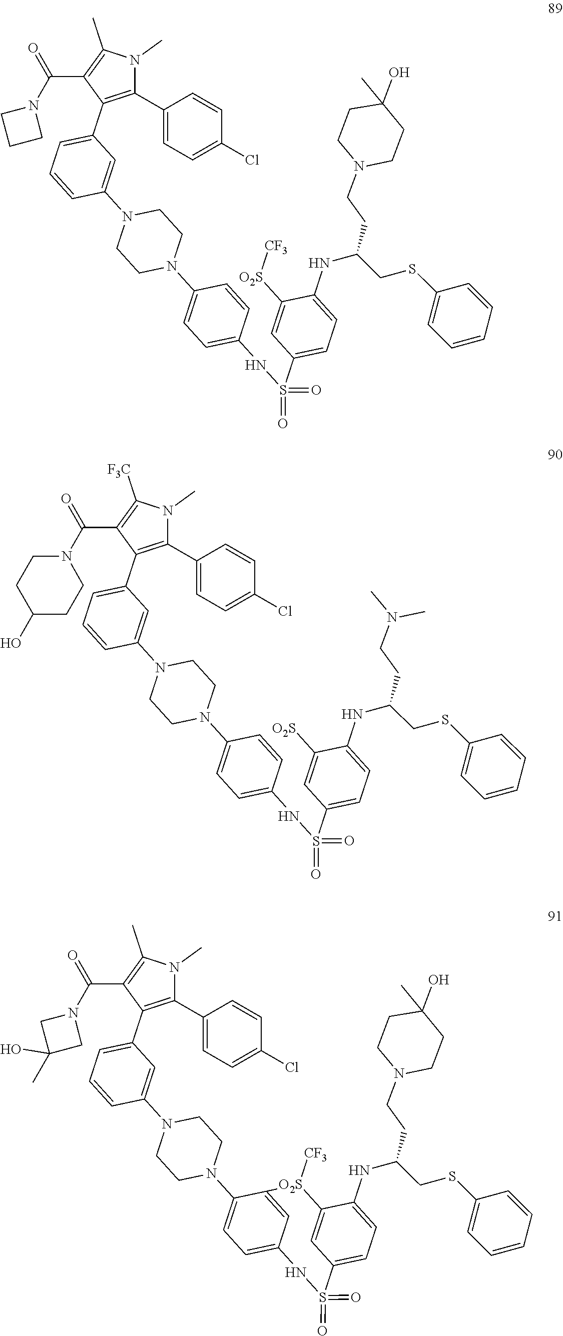

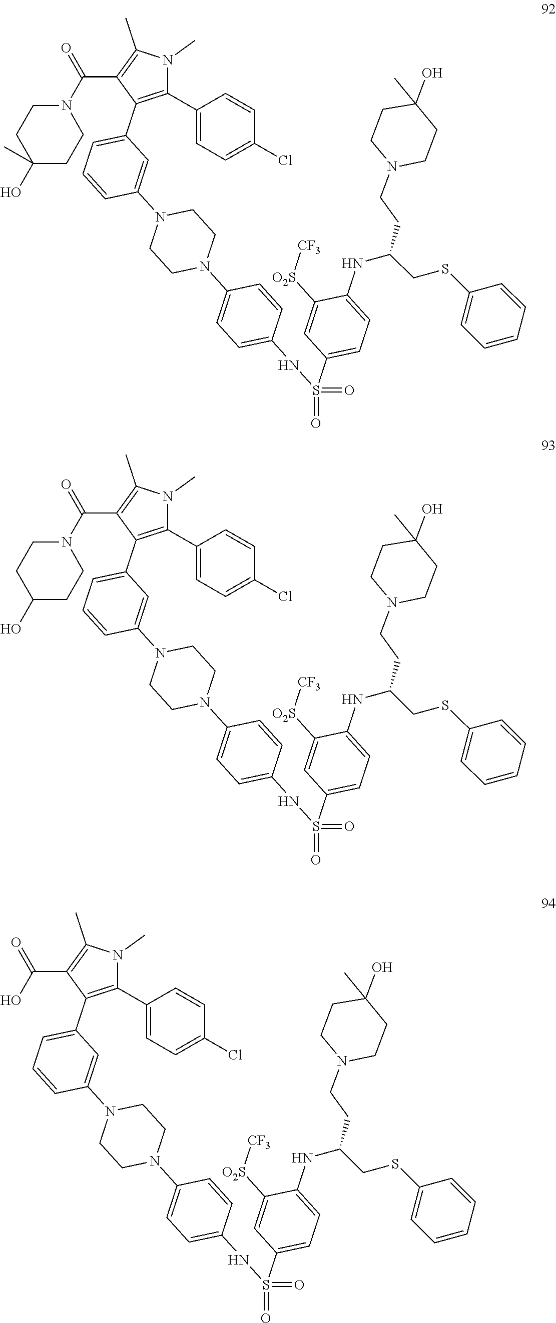 [SMILES: Cc1c(C(=O)N2CCC(C)(O)CC2)c(-c2cccc(N3CCN(c4ccc(NS(=O)(=O)c5ccc(N[C@H](CCN6CCC(C)(O)CC6)CSc6ccccc6)c(S(=O)(=O)C(F)(F)F)c5)cc4)CC3)c2)c(-c2ccc(Cl)cc2)n1C ; Cc1c(C(=O)N2CCC(O)CC2)c(-c2cccc(N3CCN(c4ccc(NS(=O)(=O)c5ccc(N[C@H](CCN6CCC(C)(O)CC6)CSc6ccccc6)c(S(=O)(=O)C(F)(F)F)c5)cc4)CC3)c2)c(-c2ccc(Cl)cc2)n1C ; Cc1c(C(=O)O)c(-c2cccc(N3CCN(c4ccc(NS(=O)(=O)c5ccc(N[C@H](CCN6CCC(C)(O)CC6)CSc6ccccc6)c(S(=O)(=O)C(F)(F)F)c5)cc4)CC3)c2)c(-c2ccc(Cl)cc2)n1C]

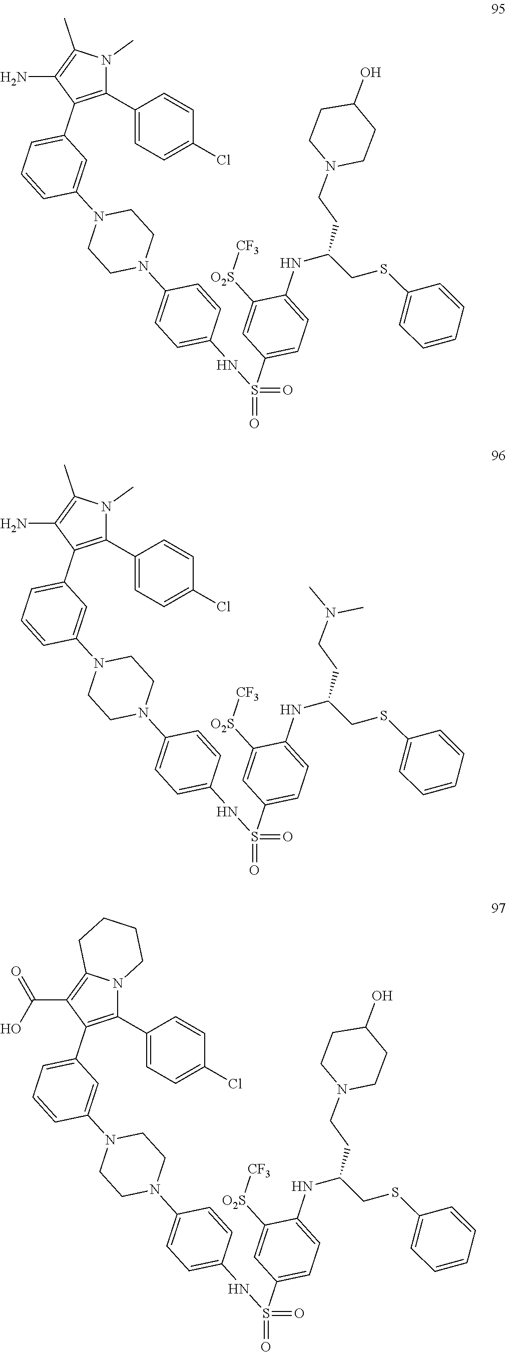 [SMILES: Cc1c(N)c(-c2cccc(N3CCN(c4ccc(NS(=O)(=O)c5ccc(N[C@H](CCN6CCC(O)CC6)CSc6ccccc6)c(S(=O)(=O)C(F)(F)F)c5)cc4)CC3)c2)c(-c2ccc(Cl)cc2)n1C ; Cc1c(N)c(-c2cccc(N3CCN(c4ccc(NS(=O)(=O)c5ccc(N[C@H](CCN(C)C)CSc6ccccc6)c(S(=O)(=O)C(F)(F)F)c5)cc4)CC3)c2)c(-c2ccc(Cl)cc2)n1C ; O=C(O)c1c(-c2cccc(N3CCN(c4ccc(NS(=O)(=O)c5ccc(N[C@H](CCN6CCC(O)CC6)CSc6ccccc6)c(S(=O)(=O)C(F)(F)F)c5)cc4)CC3)c2)c(-c2ccc(Cl)cc2)n2c1CCCC2]

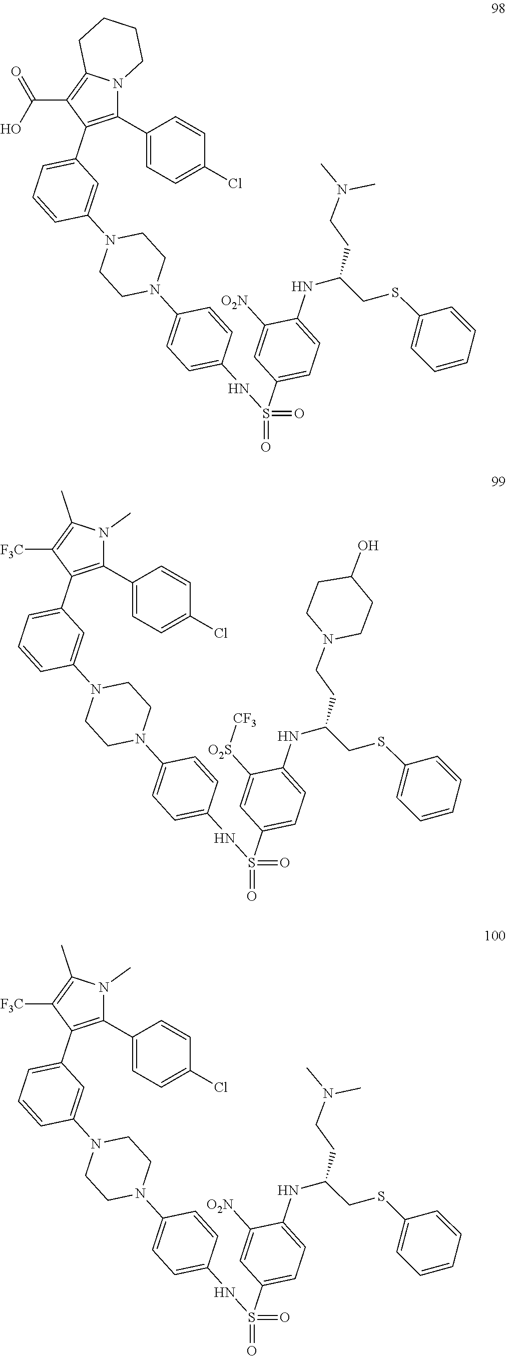 [SMILES: CN(C)CC[C@H](CSc1ccccc1)Nc1ccc(S(=O)(=O)Nc2ccc(N3CCN(c4cccc(-c5c(C(=O)O)c6n(c5-c5ccc(Cl)cc5)CCCC6)c4)CC3)cc2)cc1[N+](=O)[O-] ; Cc1c(C(F)(F)F)c(-c2cccc(N3CCN(c4ccc(NS(=O)(=O)c5ccc(N[C@H](CCN6CCC(O)CC6)CSc6ccccc6)c(S(=O)(=O)C(F)(F)F)c5)cc4)CC3)c2)c(-c2ccc(Cl)cc2)n1C ; Cc1c(C(F)(F)F)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C]

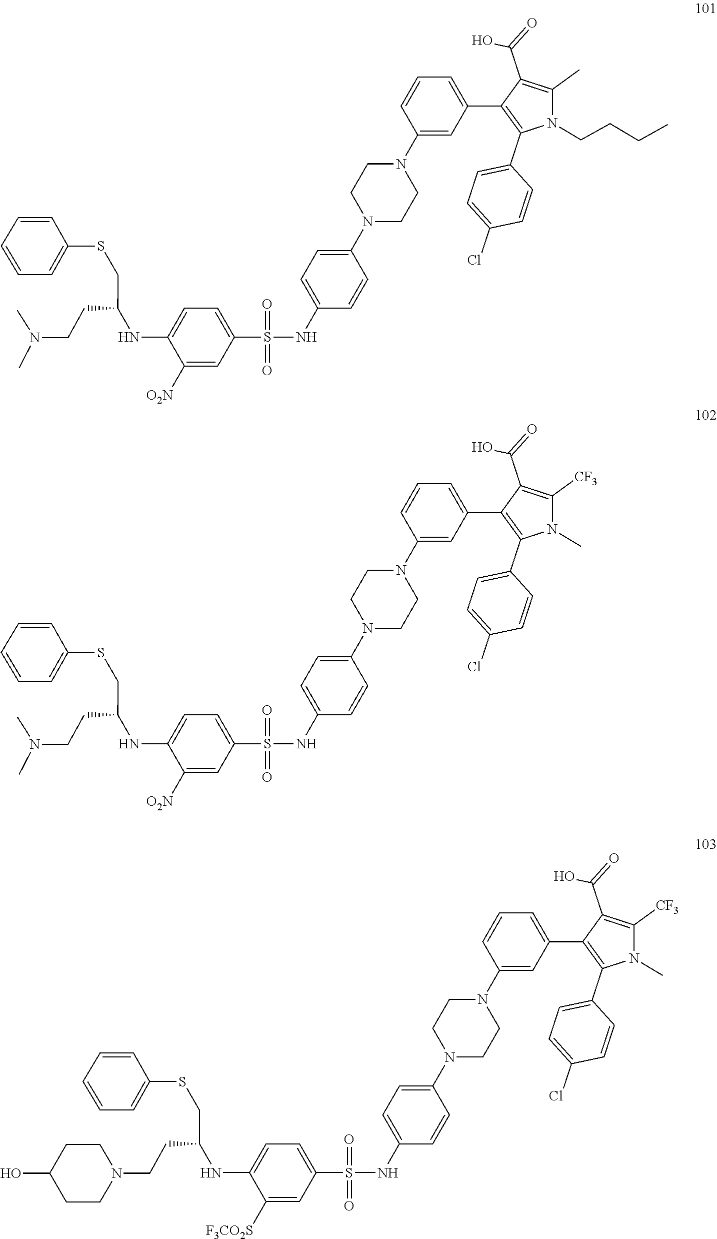

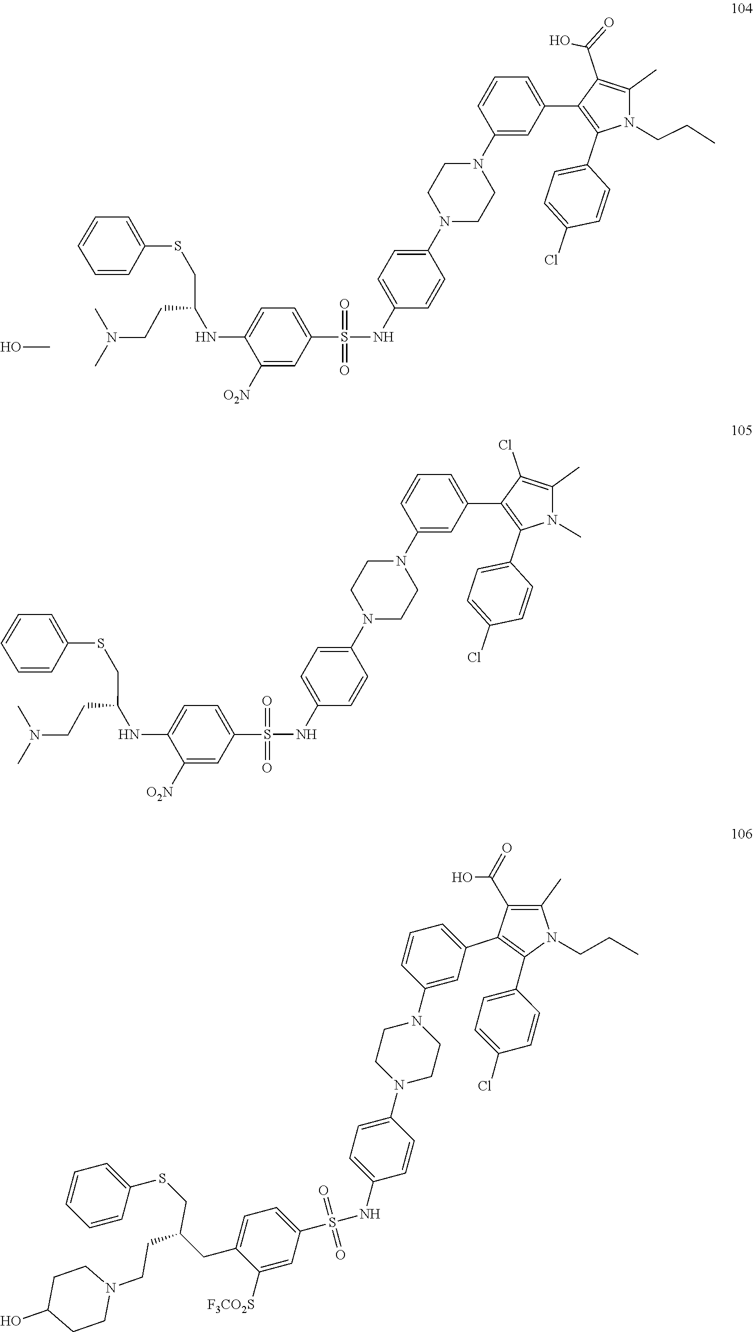

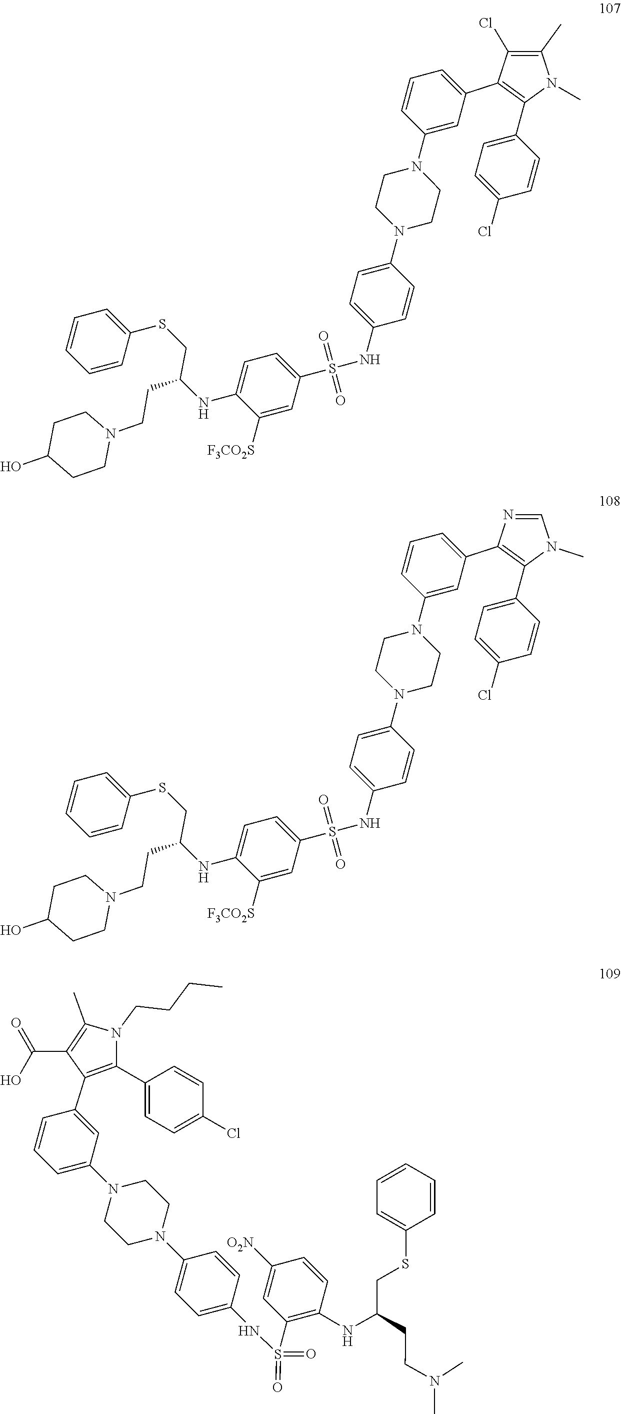

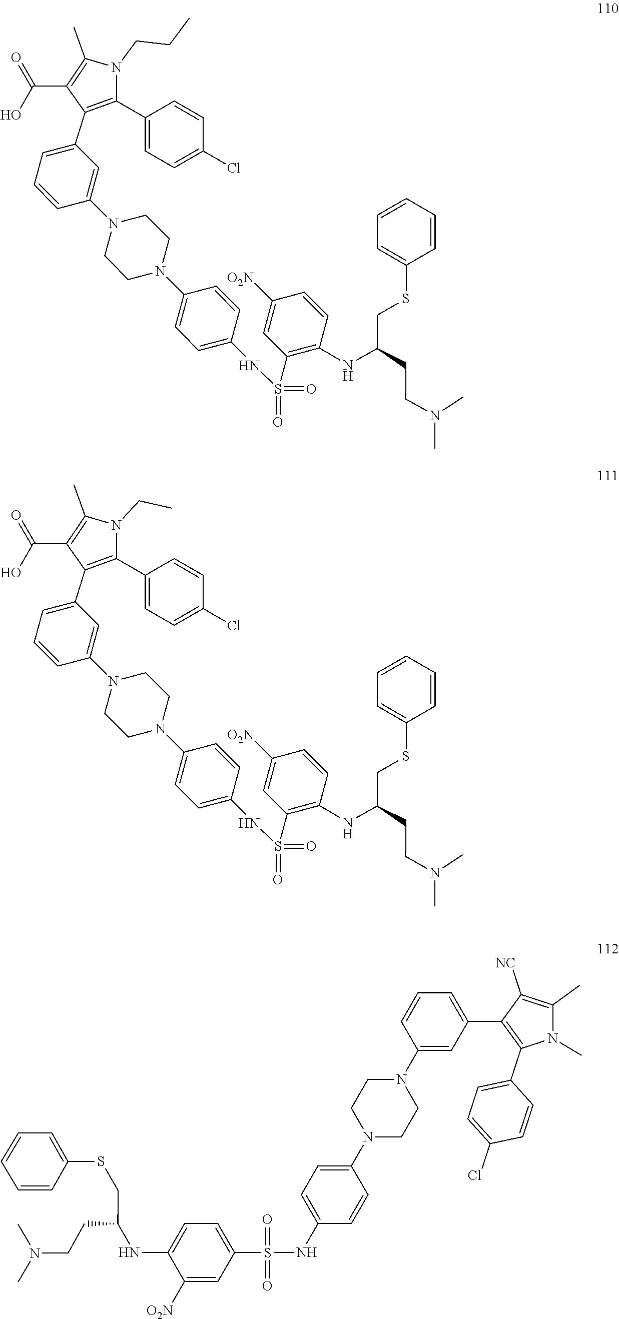 [SMILES: CCCn1c(C)c(C(=O)O)c(-c2cccc(N3CCN(c4ccc(NS(=O)(=O)c5cc([N+](=O)[O-])ccc5N[C@H](CCN(C)C)CSc5ccccc5)cc4)CC3)c2)c1-c1ccc(Cl)cc1 ; CCn1c(C)c(C(=O)O)c(-c2cccc(N3CCN(c4ccc(NS(=O)(=O)c5cc([N+](=O)[O-])ccc5N[C@H](CCN(C)C)CSc5ccccc5)cc4)CC3)c2)c1-c1ccc(Cl)cc1 ; Cc1c(C#N)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C]

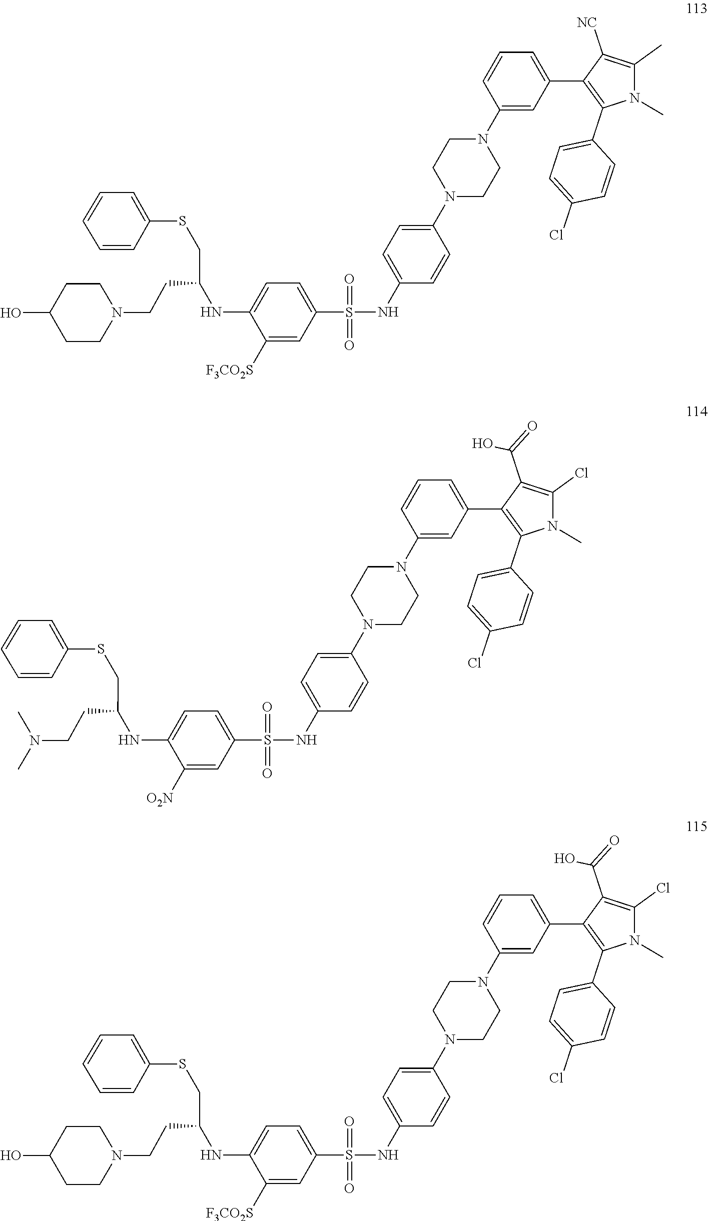

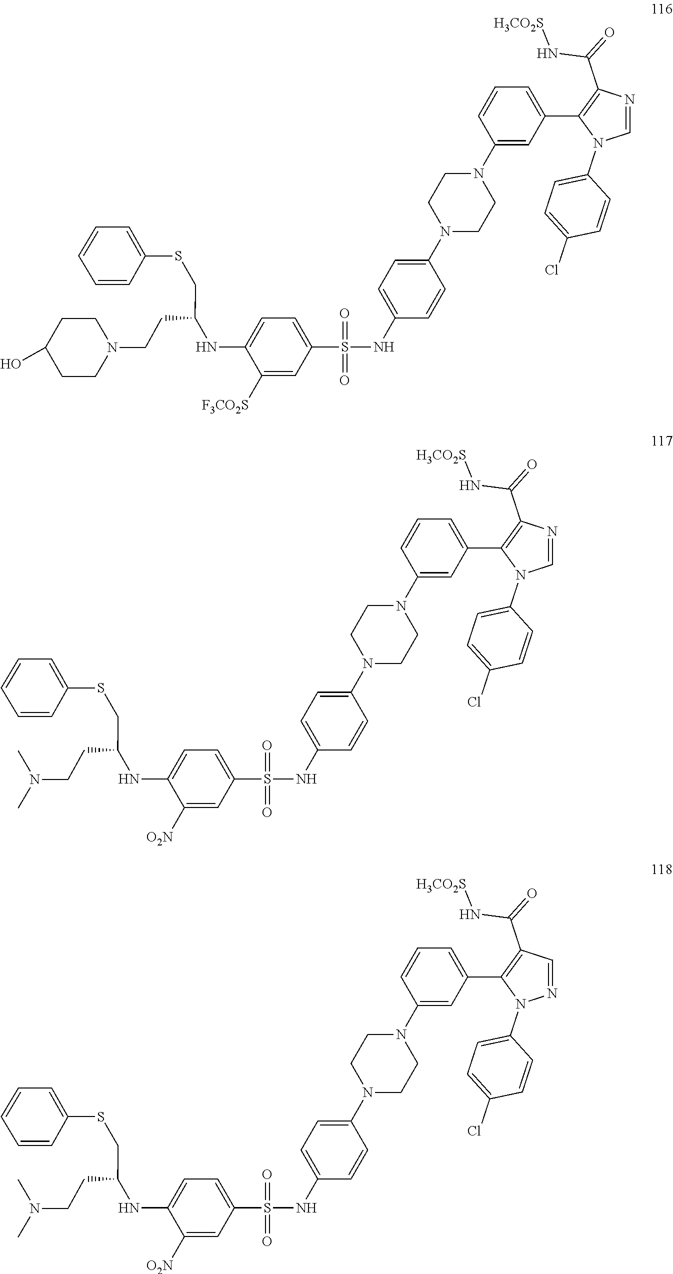

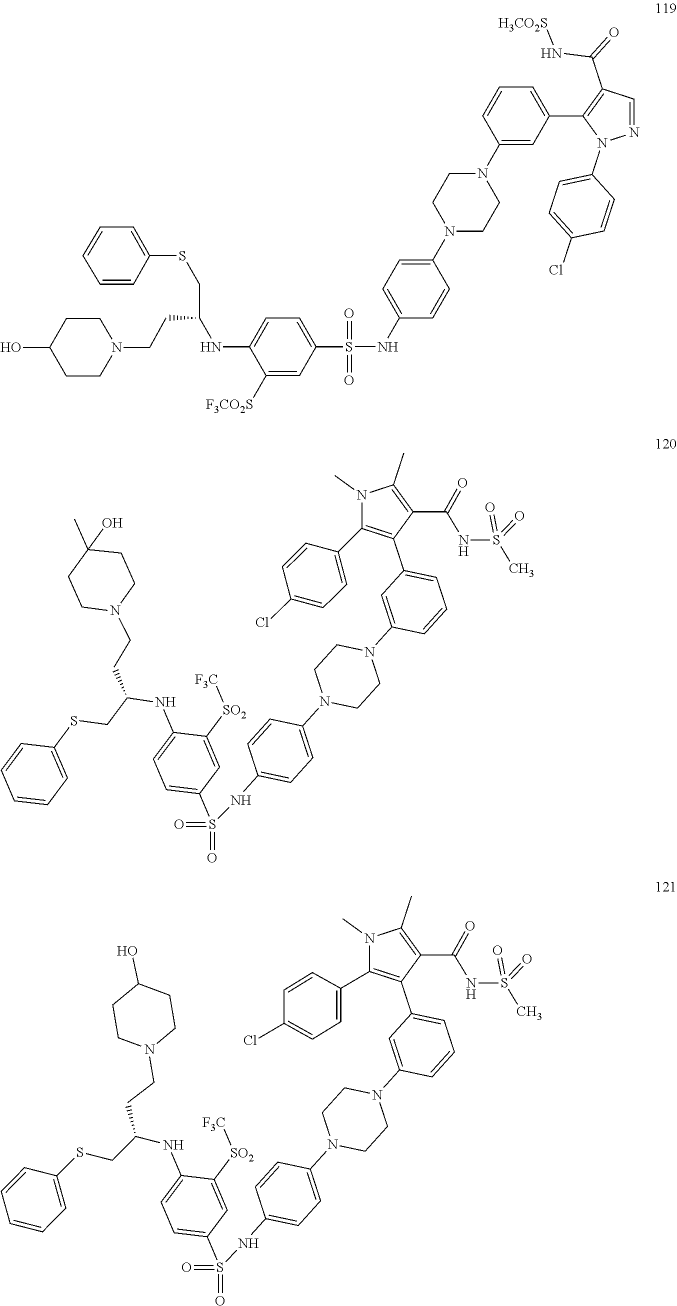

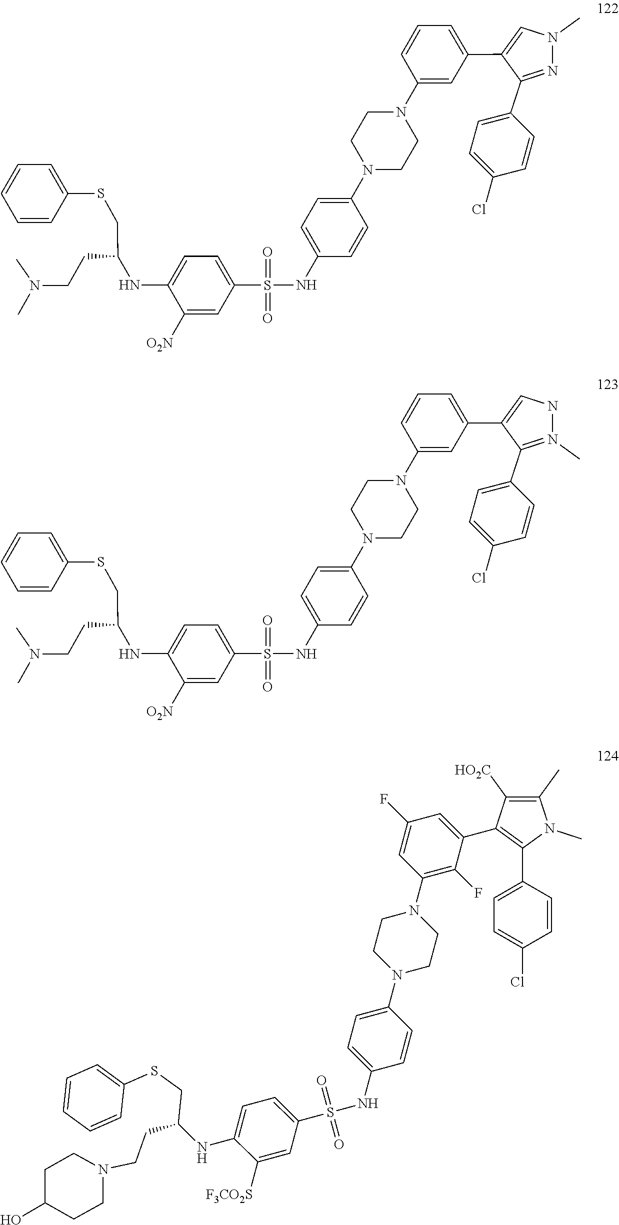

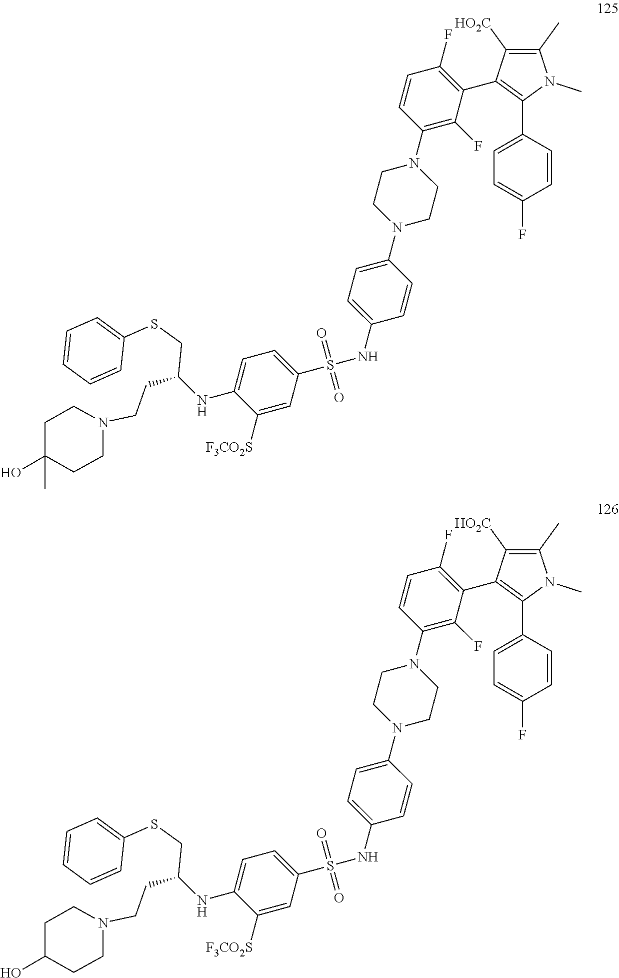

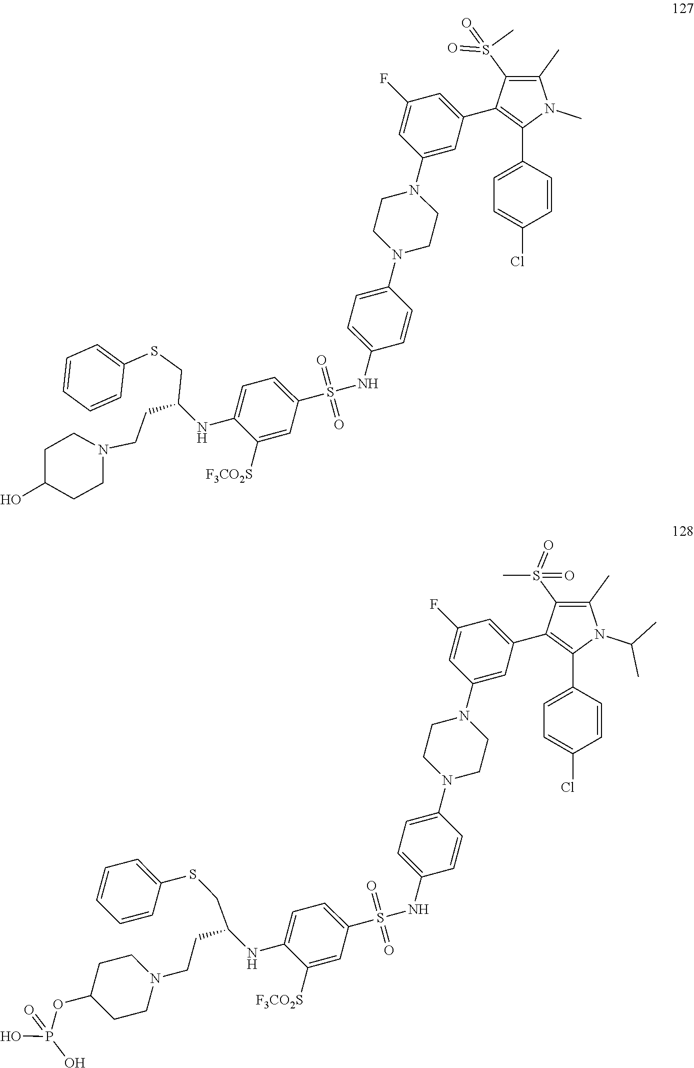

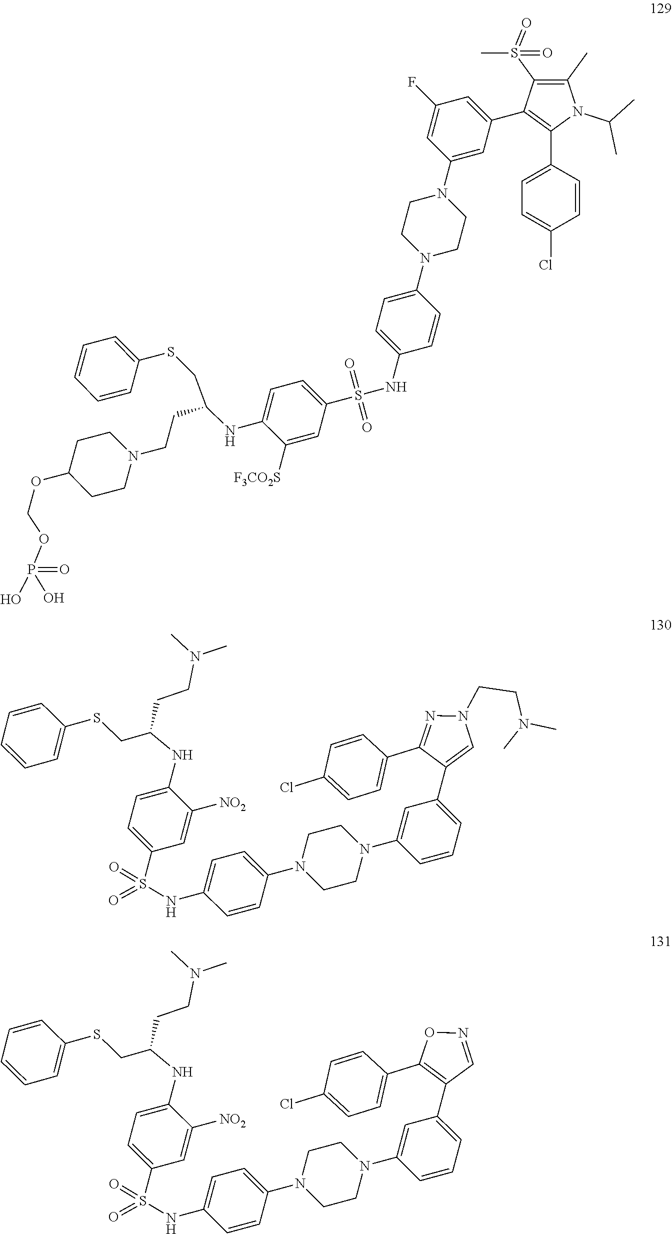

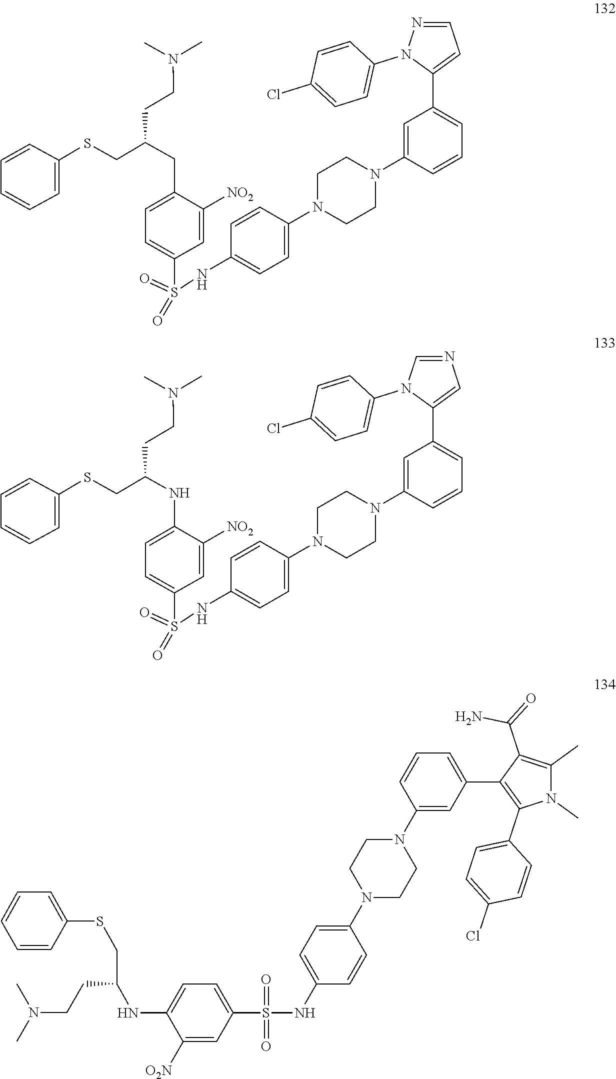 [SMILES: CN(C)CC[C@@H](CSc1ccccc1)Cc1ccc(S(=O)(=O)Nc2ccc(N3CCN(c4cccc(-c5ccnn5-c5ccc(Cl)cc5)c4)CC3)cc2)cc1[N+](=O)[O-] ; CN(C)CC[C@@H](CSc1ccccc1)Nc1ccc(S(=O)(=O)Nc2ccc(N3CCN(c4cccc(-c5cncn5-c5ccc(Cl)cc5)c4)CC3)cc2)cc1[N+](=O)[O-] ; Cc1c(C(N)=O)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C]

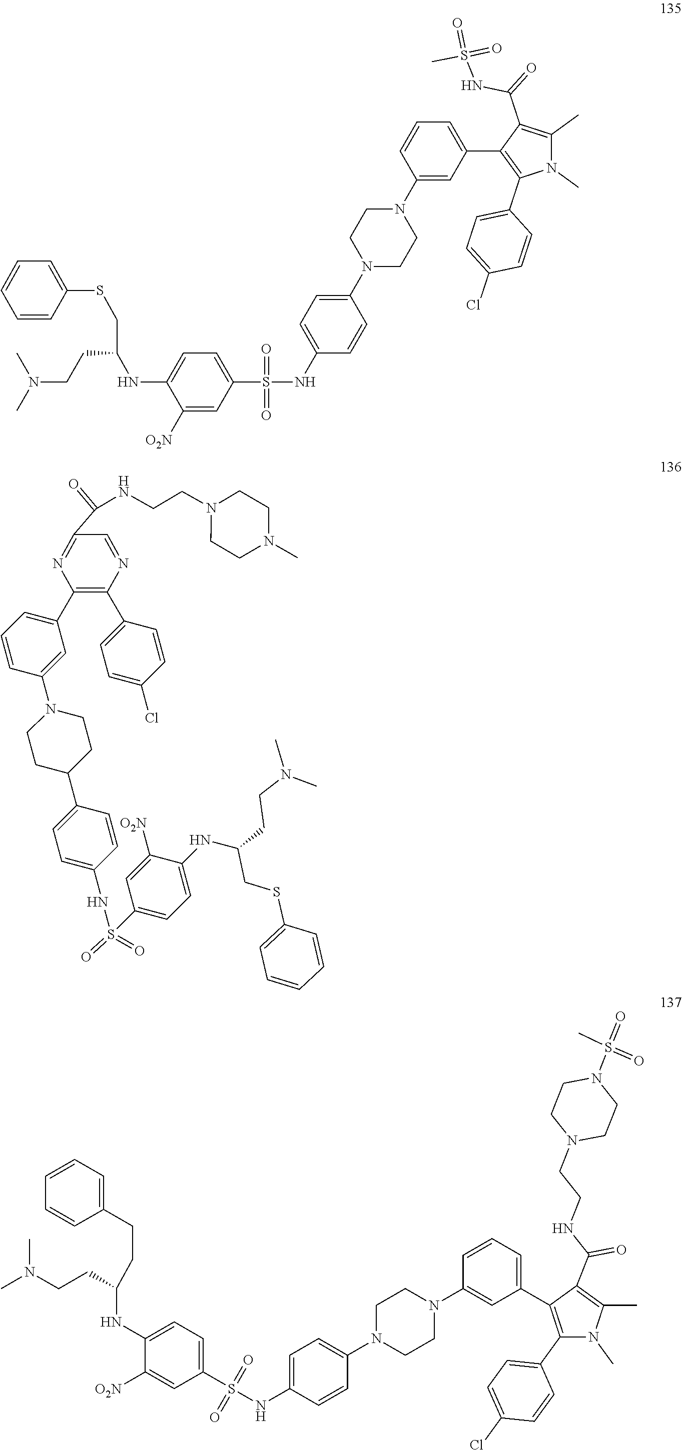

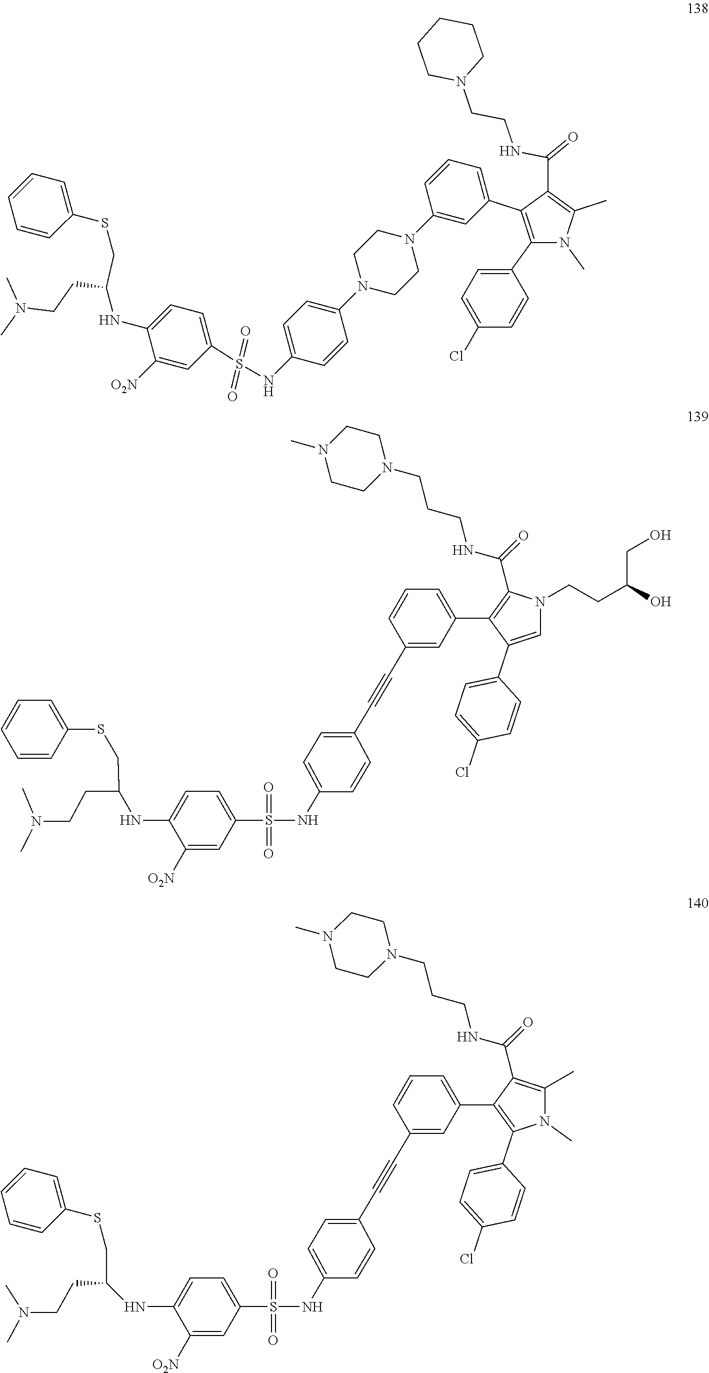

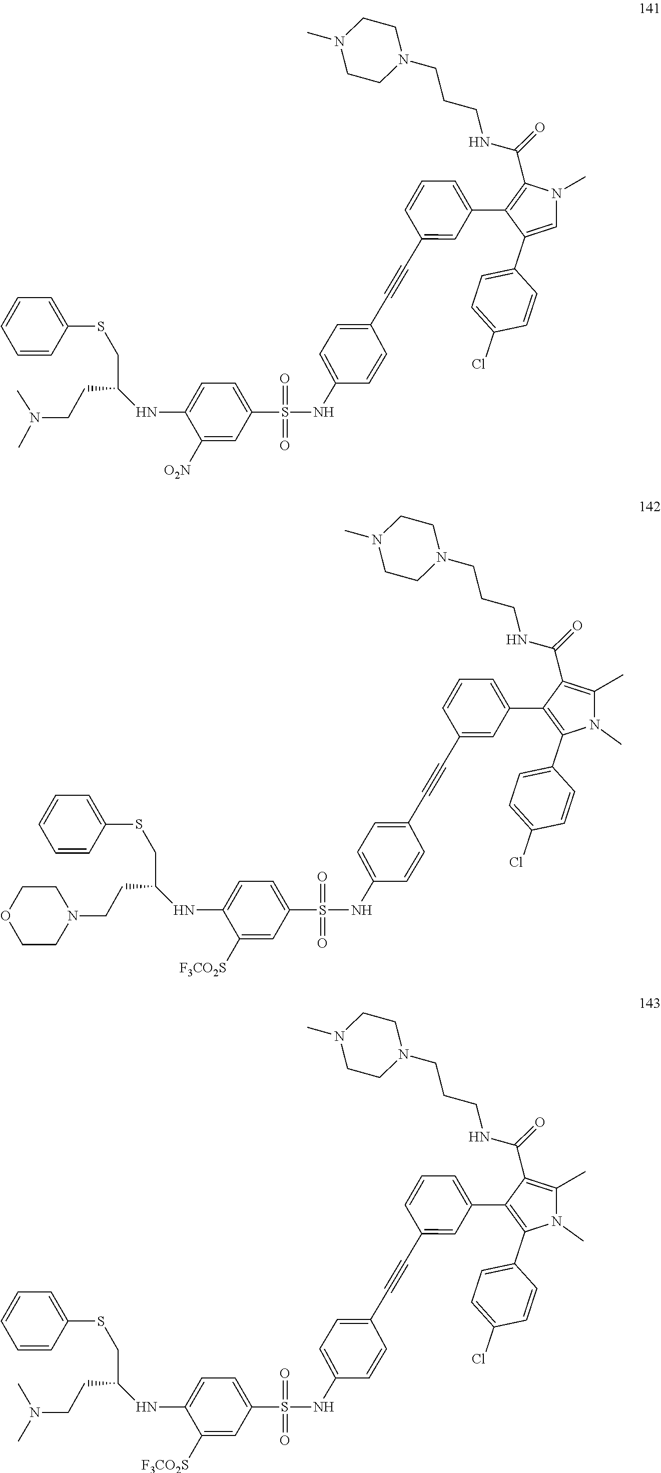

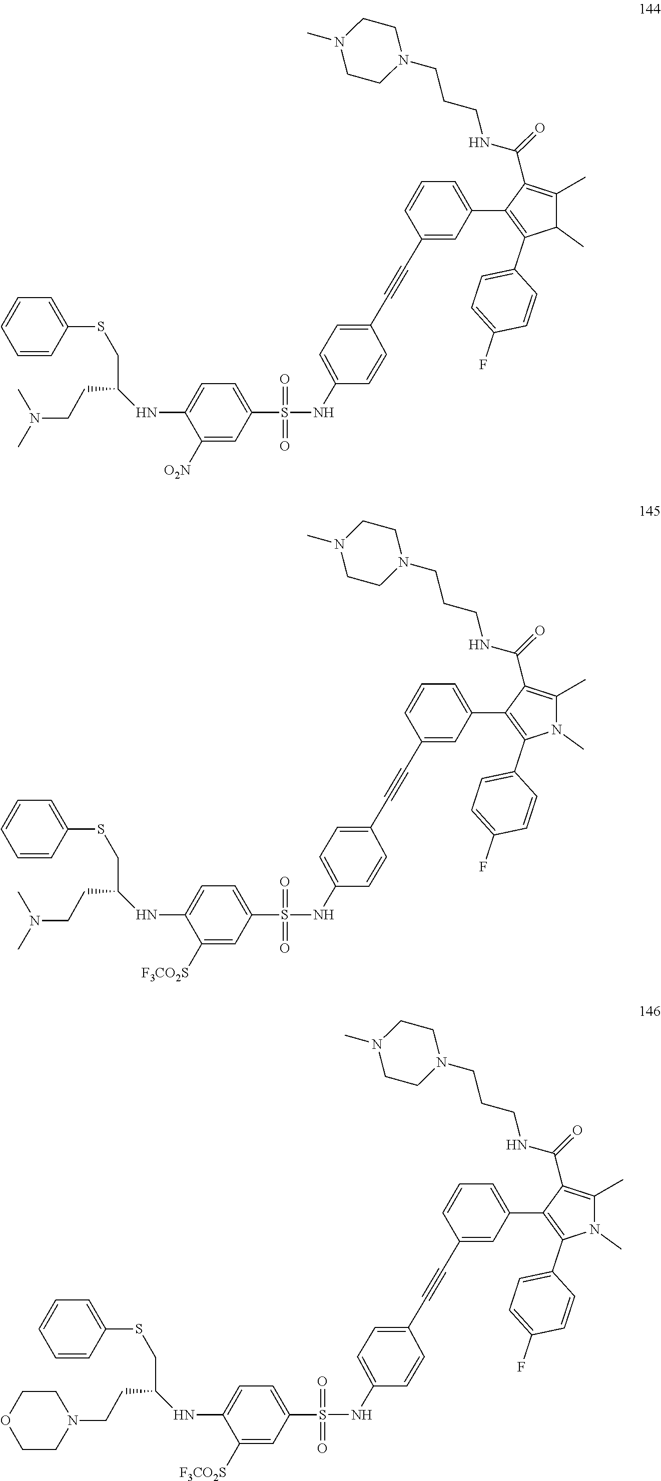

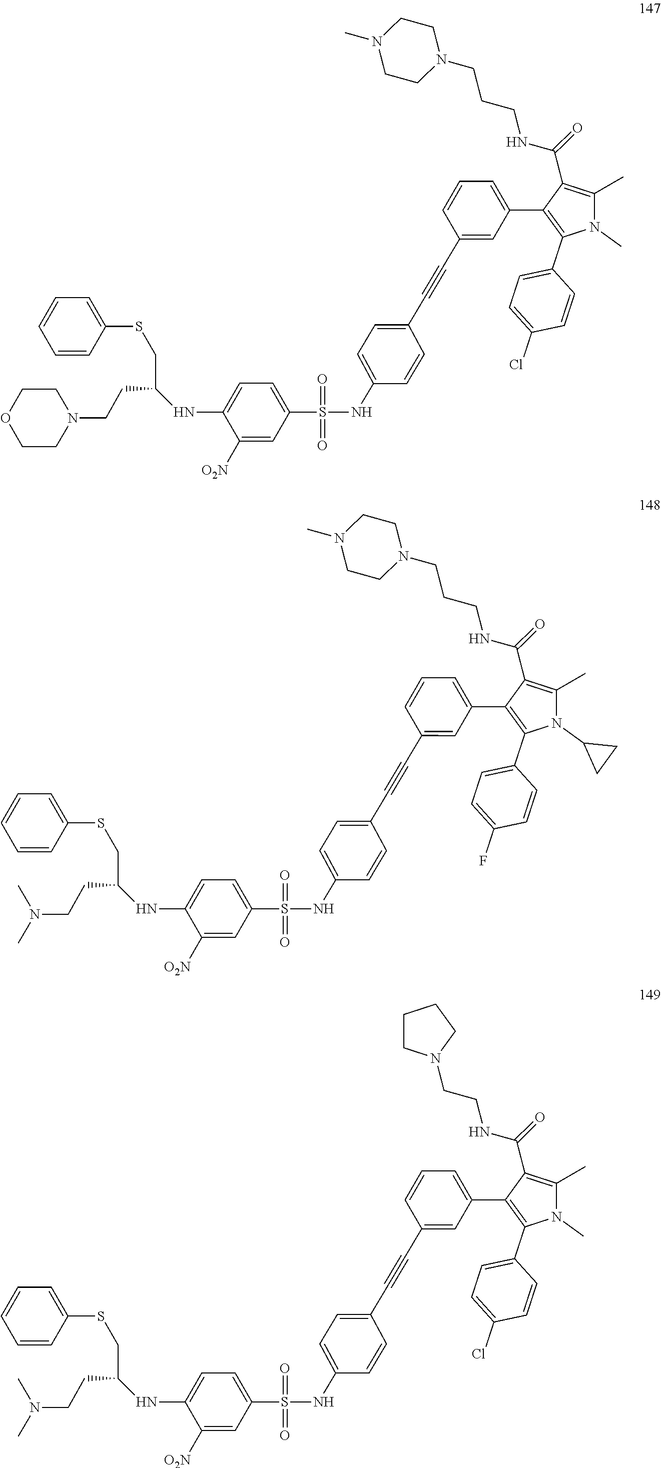 [SMILES: Cc1c(C(=O)NCCCN2CCN(C)CC2)c(-c2cccc(C#Cc3ccc(NS(=O)(=O)c4ccc(N[C@H](CCN5CCOCC5)CSc5ccccc5)c([N+](=O)[O-])c4)cc3)c2)c(-c2ccc(Cl)cc2)n1C ; Cc1c(C(=O)NCCCN2CCN(C)CC2)c(-c2cccc(C#Cc3ccc(NS(=O)(=O)c4ccc(N[C@H](CCN(C)C)CSc5ccccc5)c([N+](=O)[O-])c4)cc3)c2)c(-c2ccc(F)cc2)n1C1CC1 ; Cc1c(C(=O)NCCN2CCCC2)c(-c2cccc(C#Cc3ccc(NS(=O)(=O)c4ccc(N[C@H](CCN(C)C)CSc5ccccc5)c([N+](=O)[O-])c4)cc3)c2)c(-c2ccc(Cl)cc2)n1C]

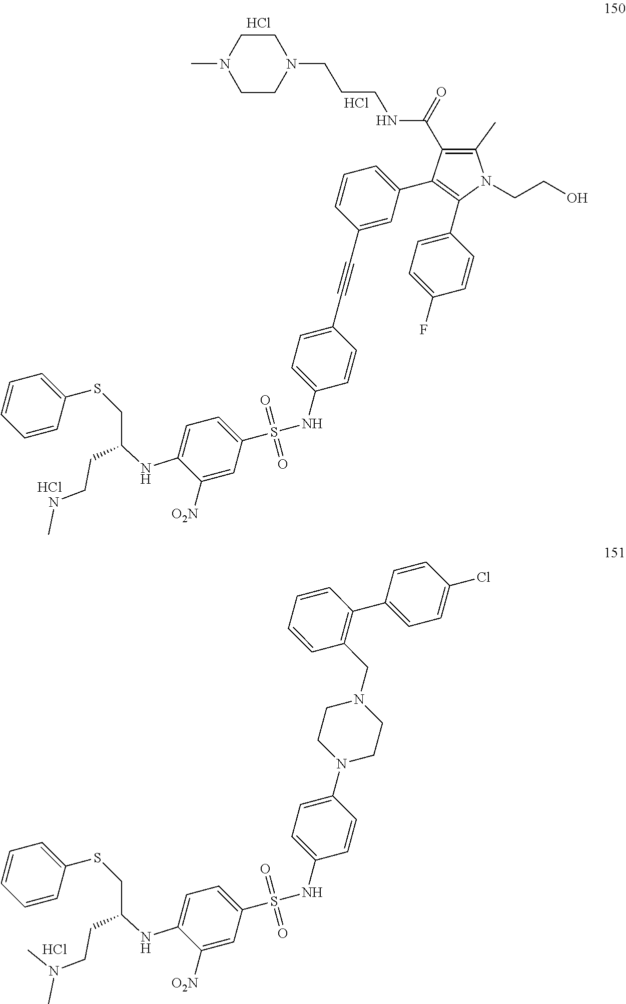 [SMILES: Cl ; CNCC[C@H](CSc1ccccc1)Nc1ccc(S(=O)(=O)Nc2ccc(C#Cc3cccc(-c4c(C(=O)NCCCN5CCN(C)CC5)c(C)n(CCO)c4-c4ccc(F)cc4)c3)cc2)cc1[N+](=O)[O-] ; Cl ; Cl ; CN(C)CC[C@H](CSc1ccccc1)Nc1ccc(S(=O)(=O)Nc2ccc(N3CCN(Cc4ccccc4-c4ccc(Cl)cc4)CC3)cc2)cc1[N+](=O)[O-] ; Cl]

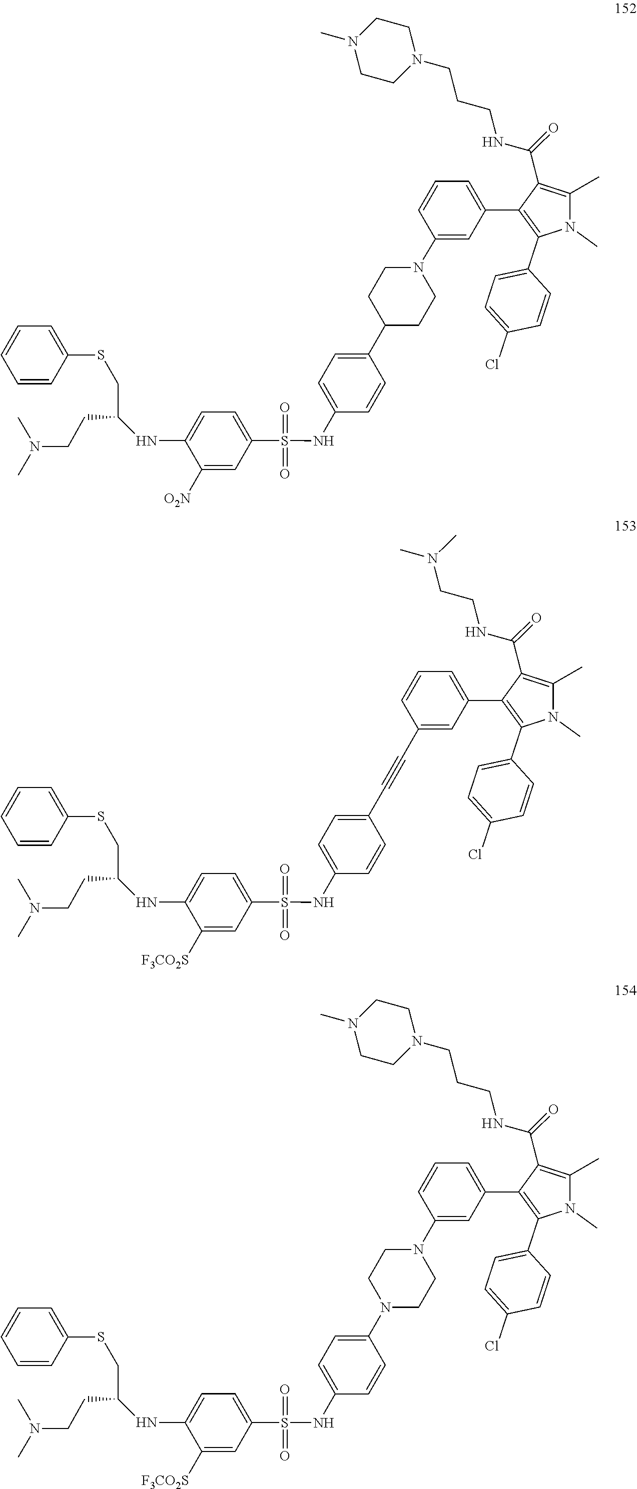

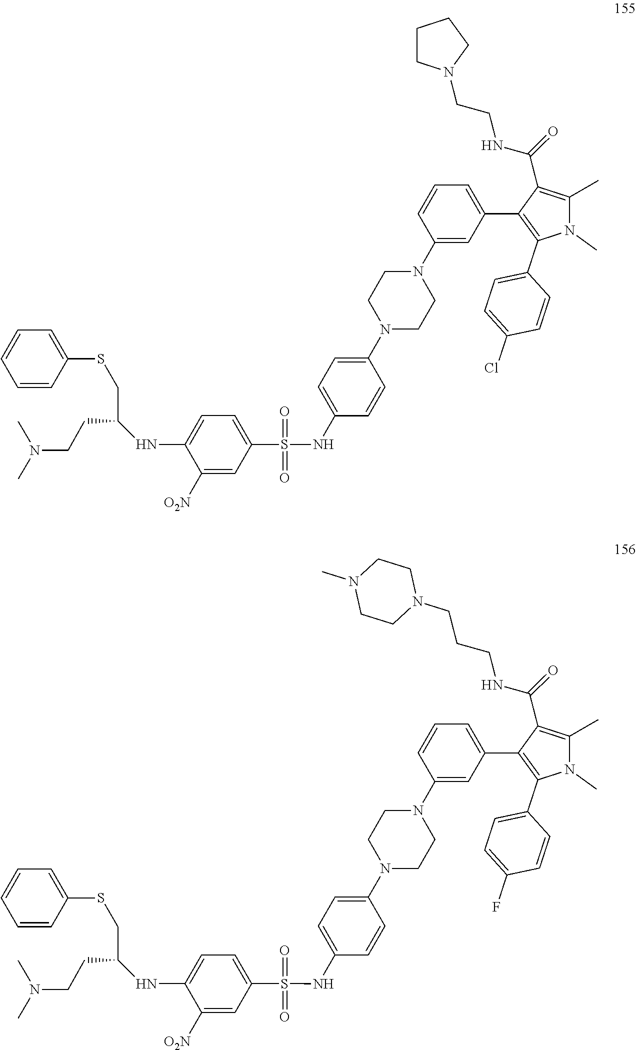 [SMILES: Cc1c(C(=O)NCCN2CCCC2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C ; Cc1c(C(=O)NCCCN2CCN(C)CC2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(F)cc2)n1C]

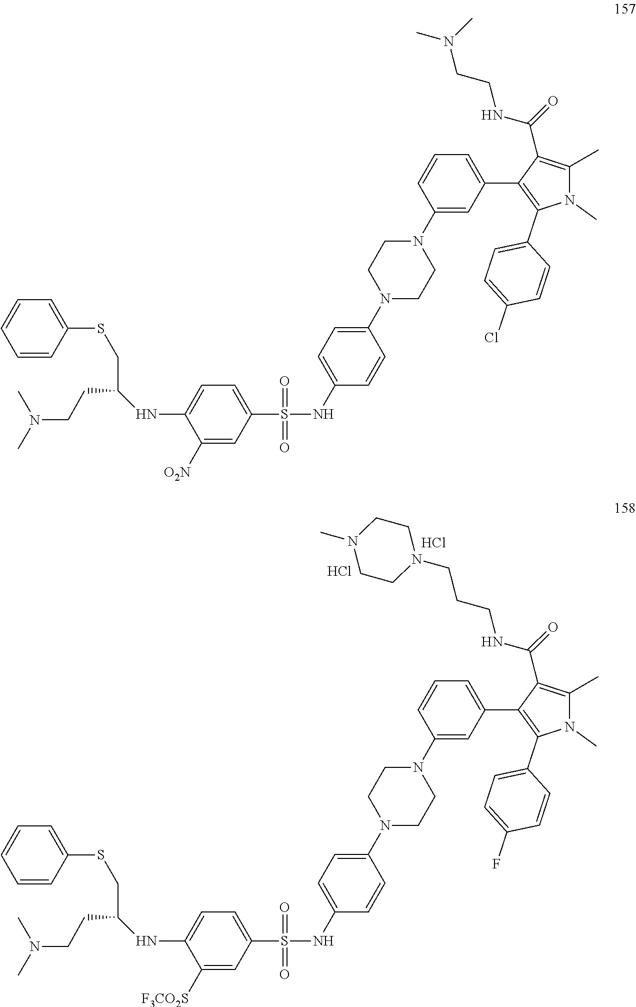

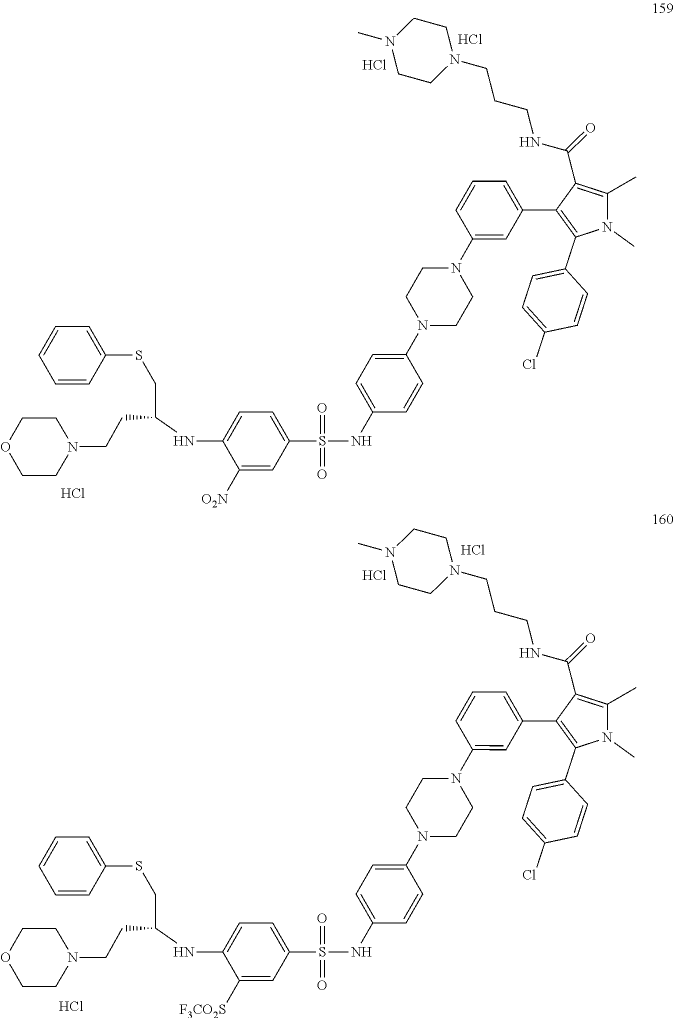

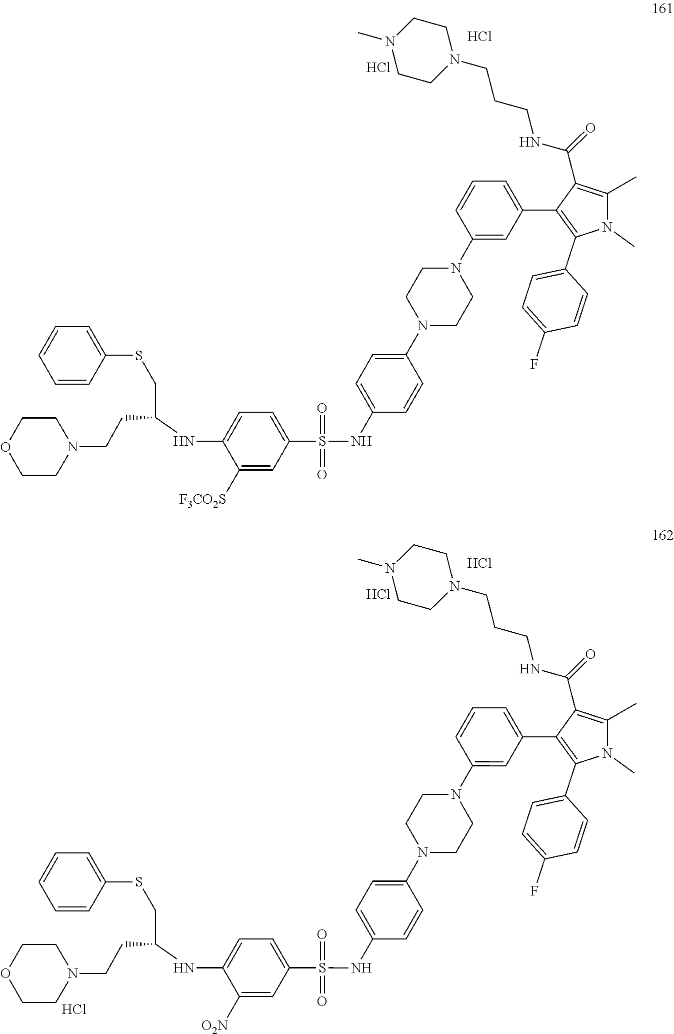

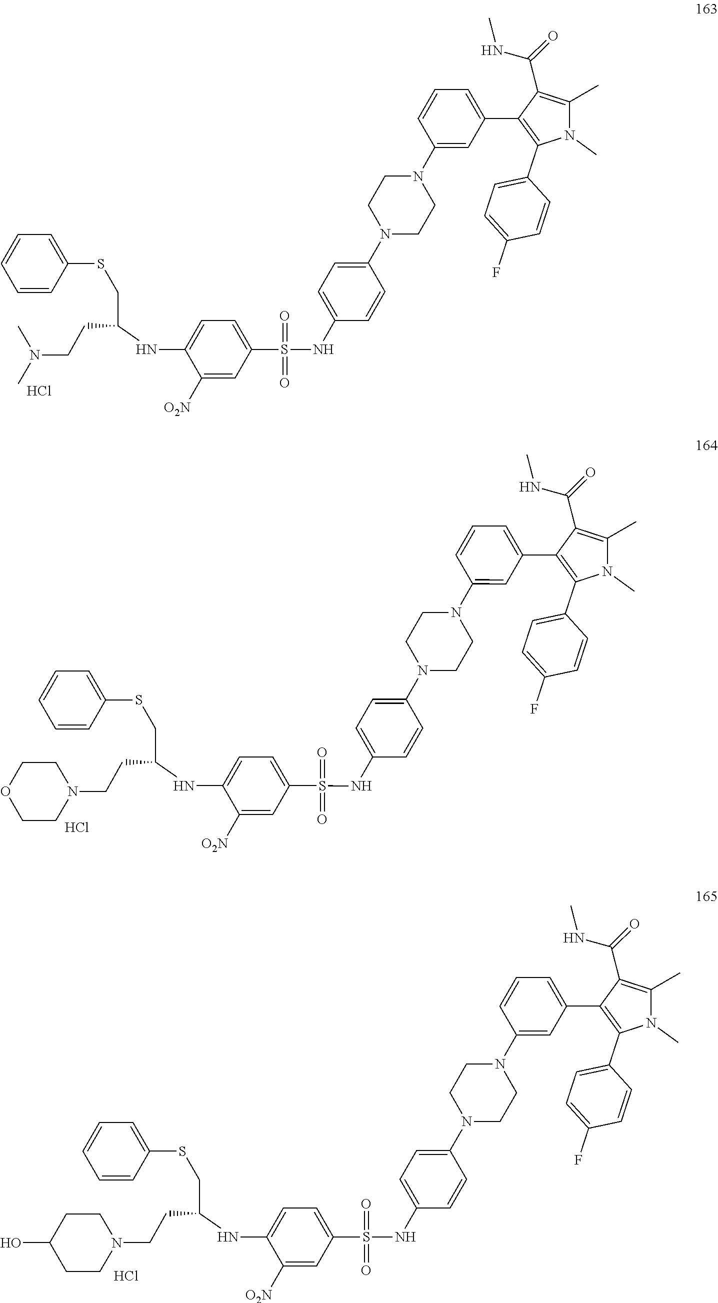 [SMILES: CNC(=O)c1c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(F)cc2)n(C)c1C ; Cl ; CNC(=O)c1c(-c2cccc(N3CCN(c4ccc(NS(=O)(=O)c5ccc(N[C@H](CCN6CCOCC6)CSc6ccccc6)c([N+](=O)[O-])c5)cc4)CC3)c2)c(-c2ccc(F)cc2)n(C)c1C ; Cl ; CNC(=O)c1c(-c2cccc(N3CCN(c4ccc(NS(=O)(=O)c5ccc(N[C@H](CCN6CCC(O)CC6)CSc6ccccc6)c([N+](=O)[O-])c5)cc4)CC3)c2)c(-c2ccc(F)cc2)n(C)c1C ; Cl]

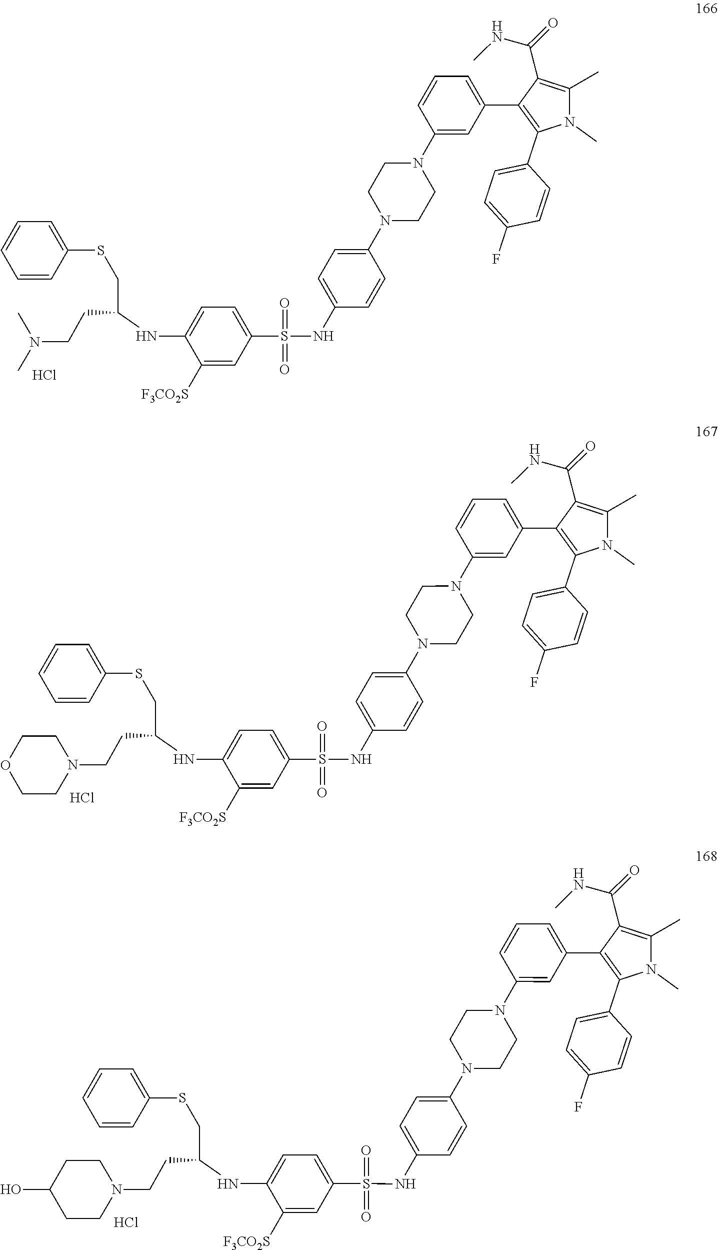

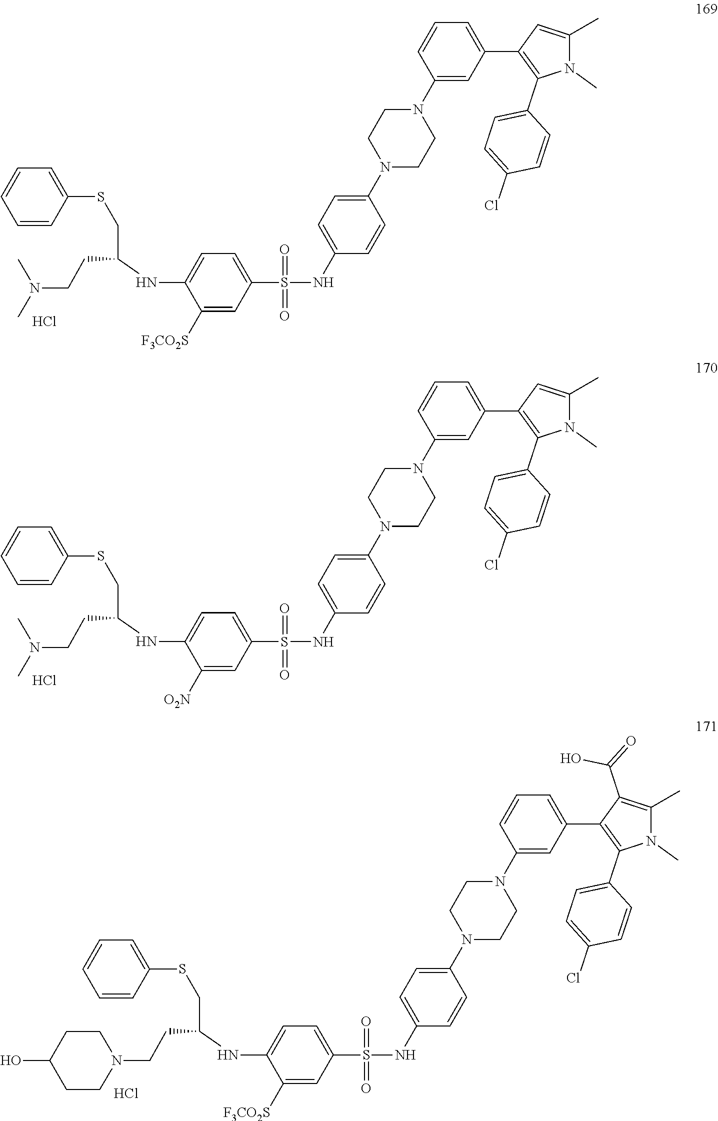

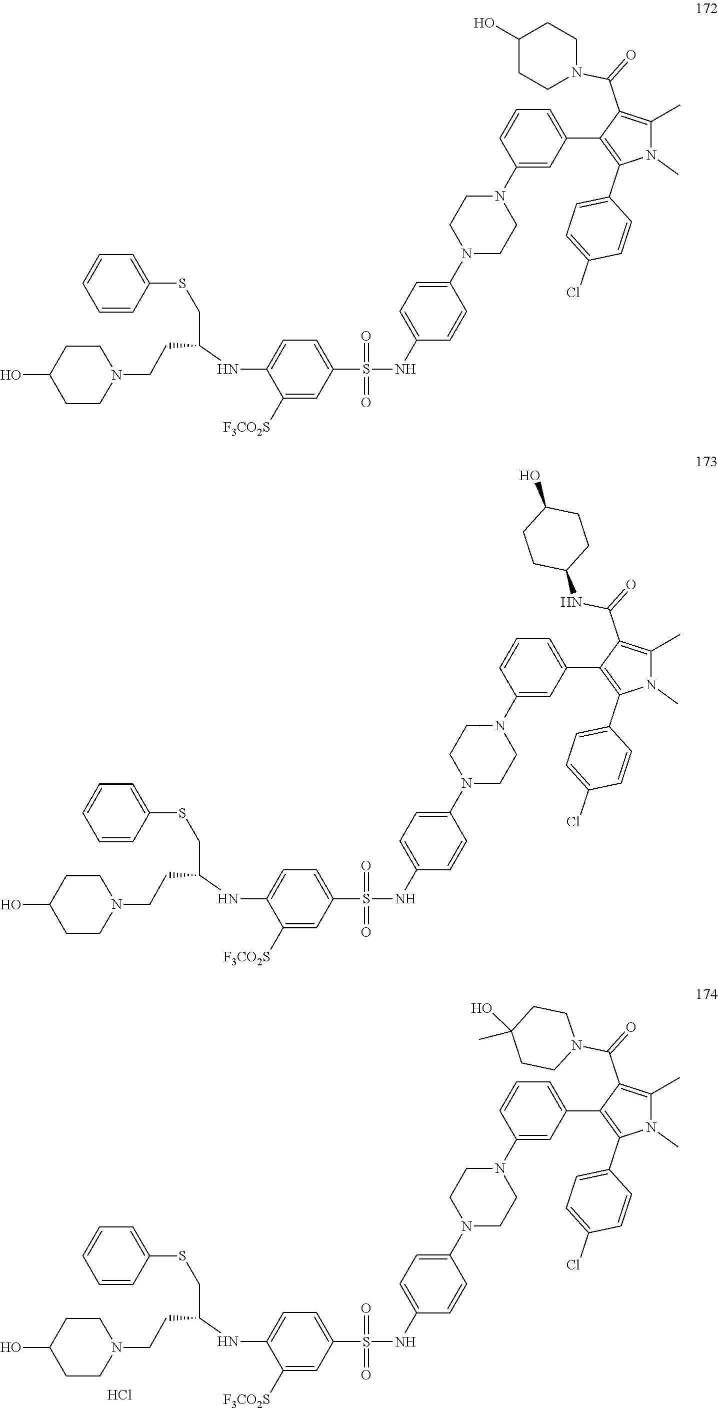

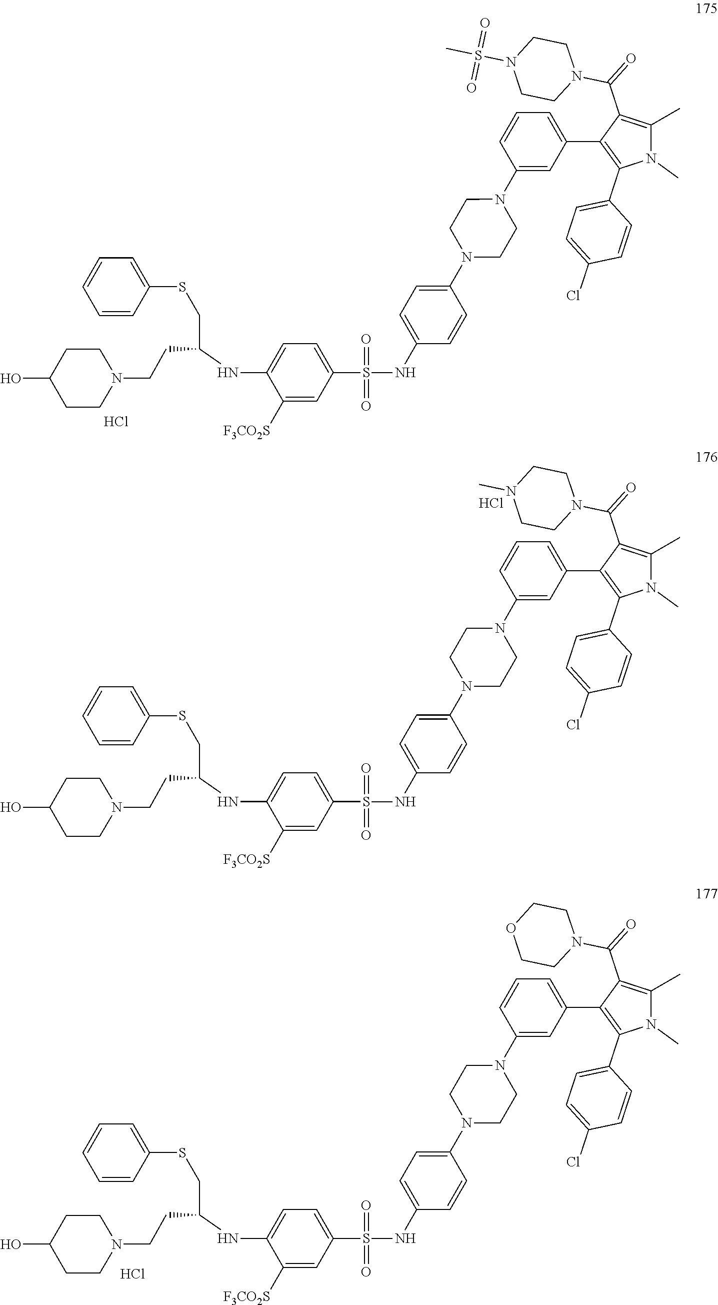

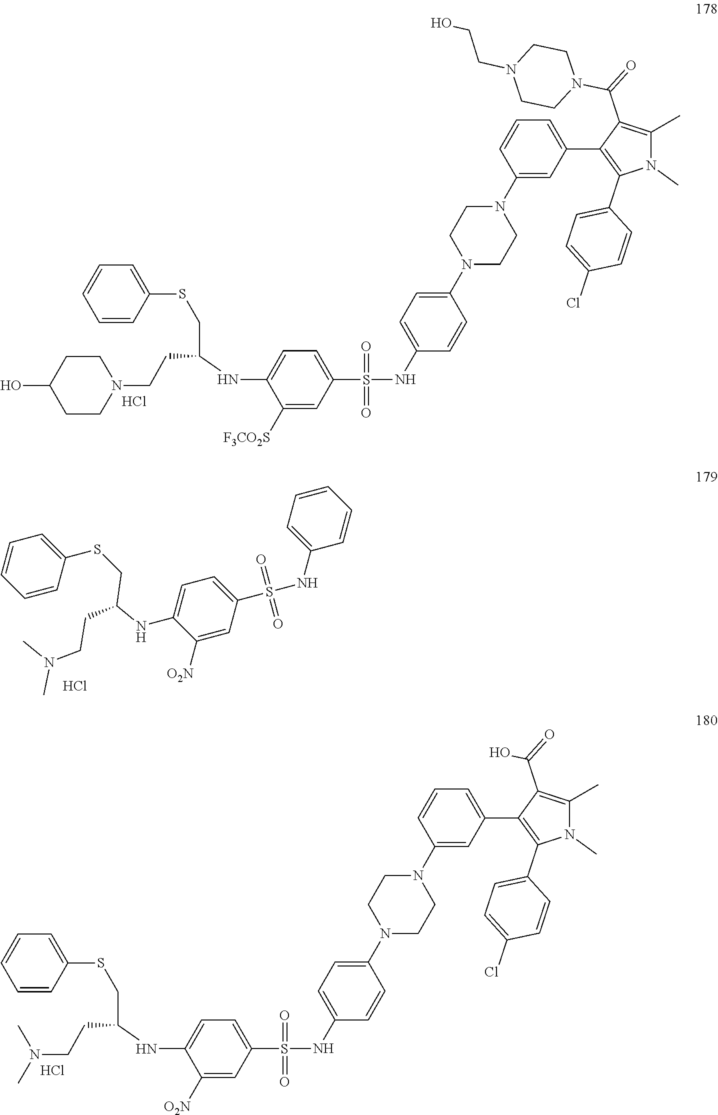

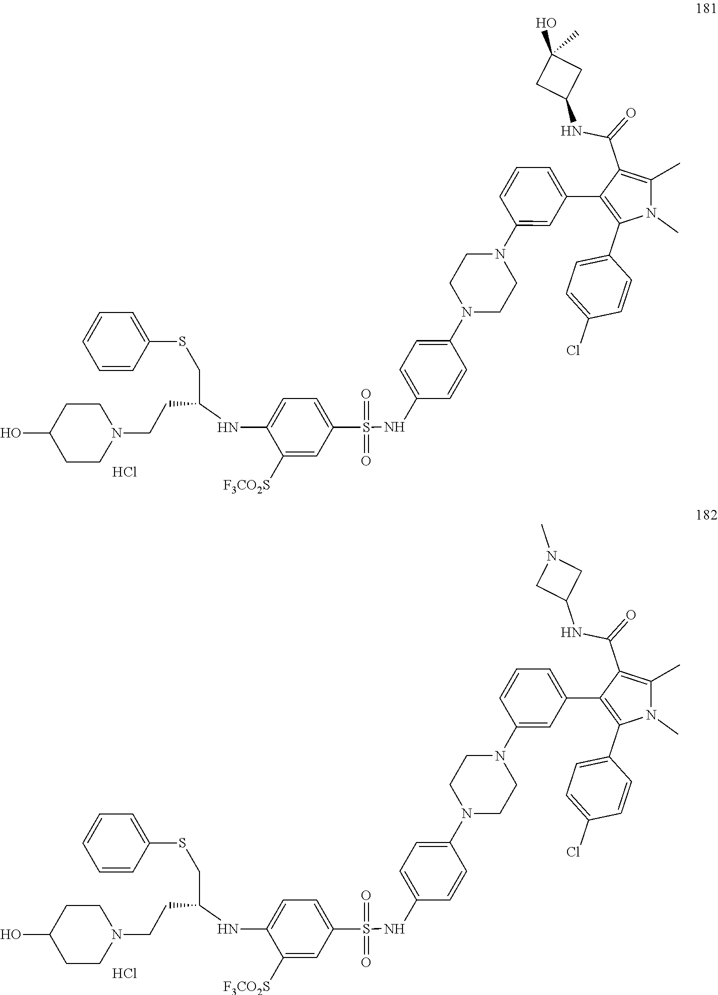

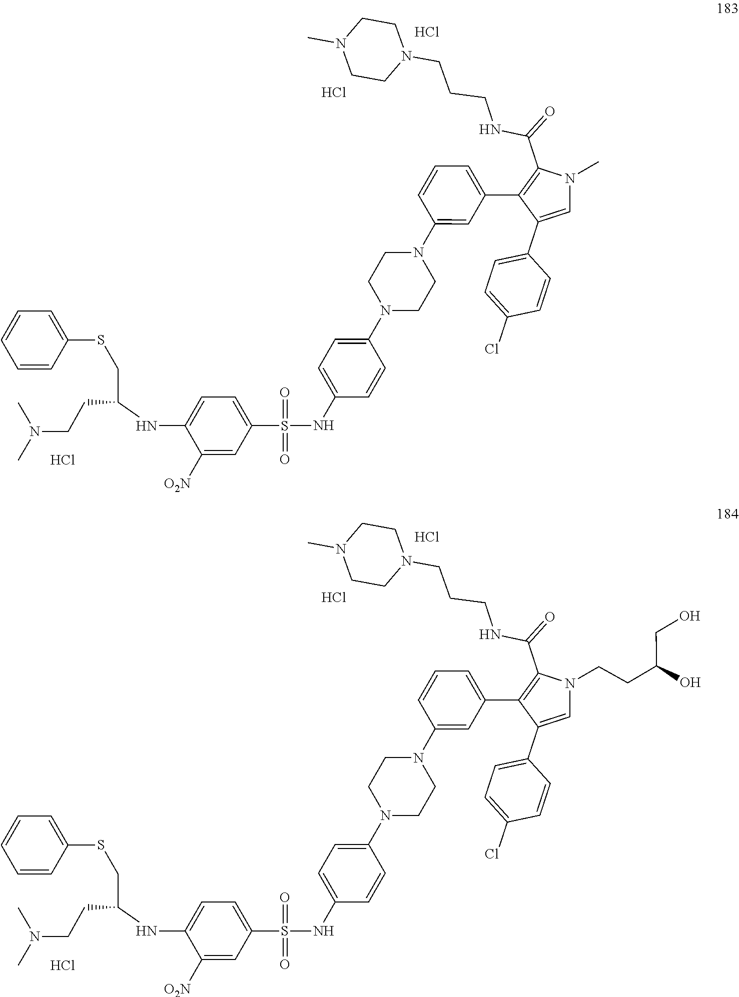

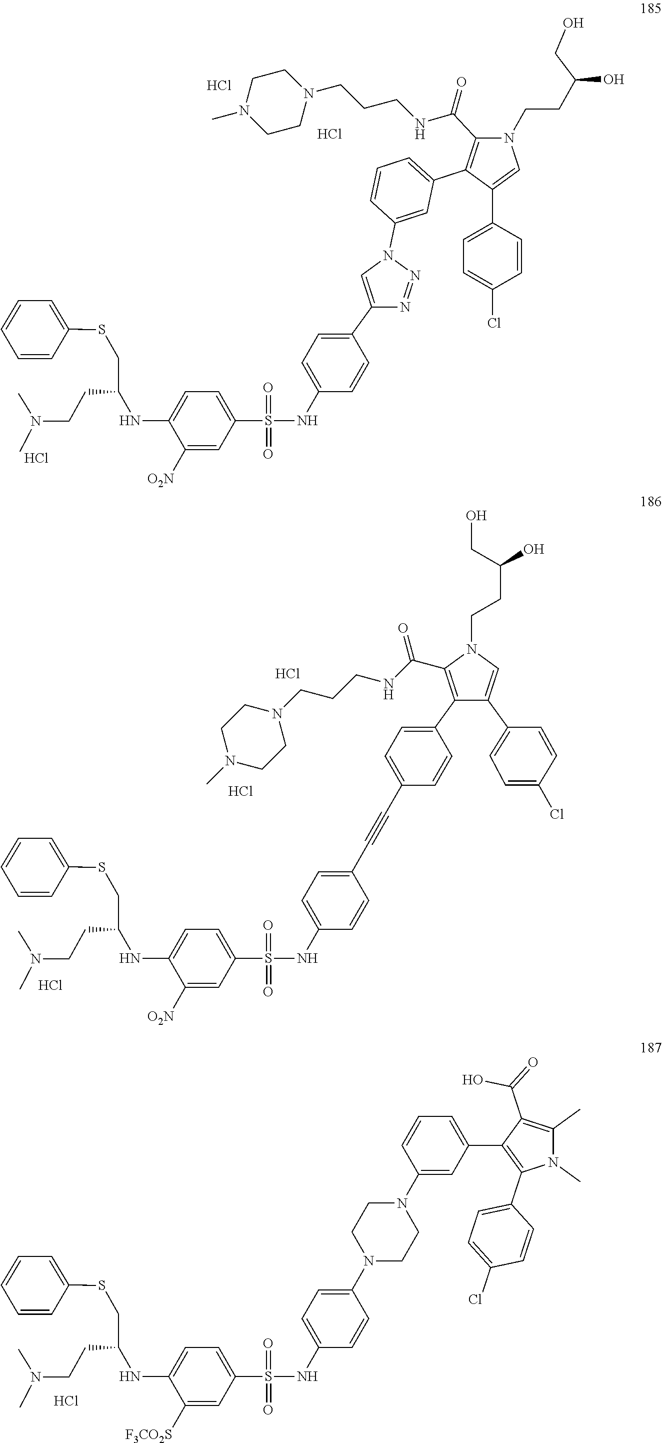

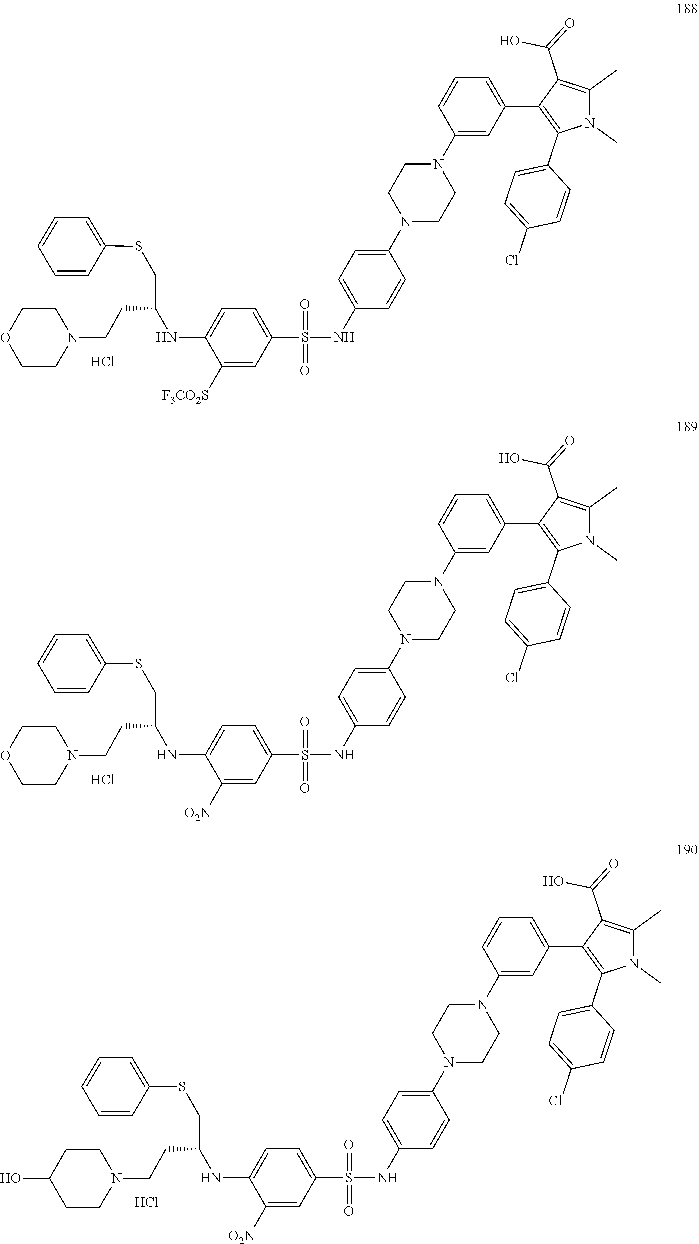

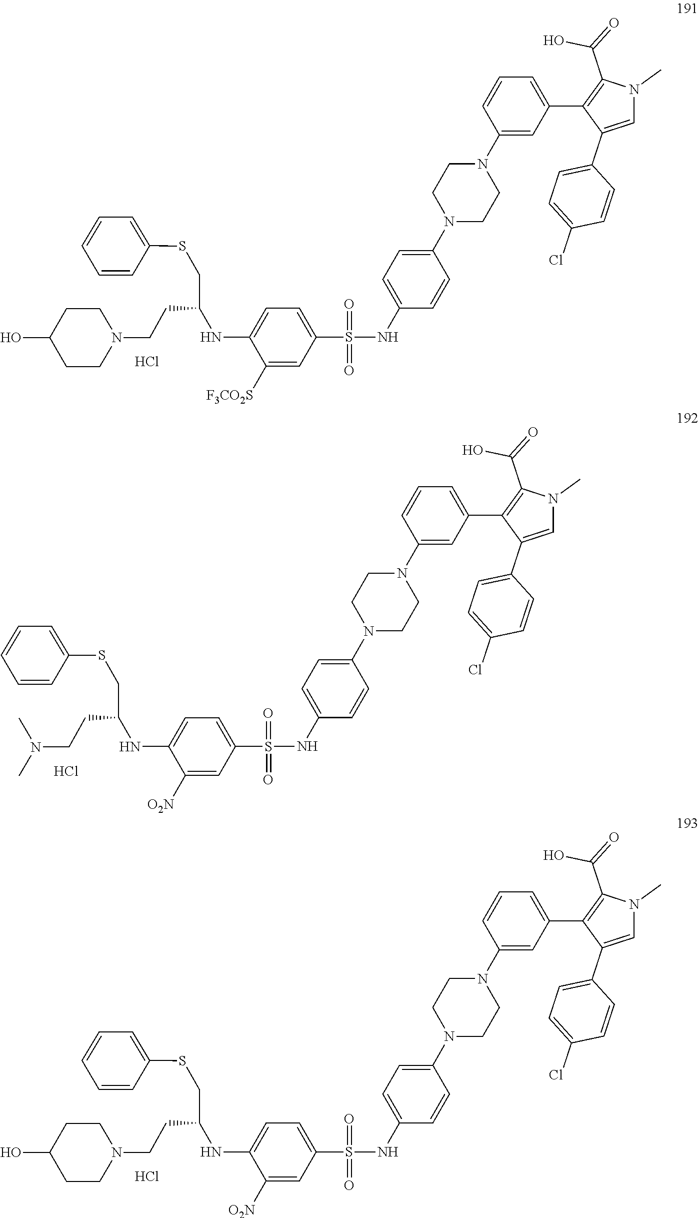

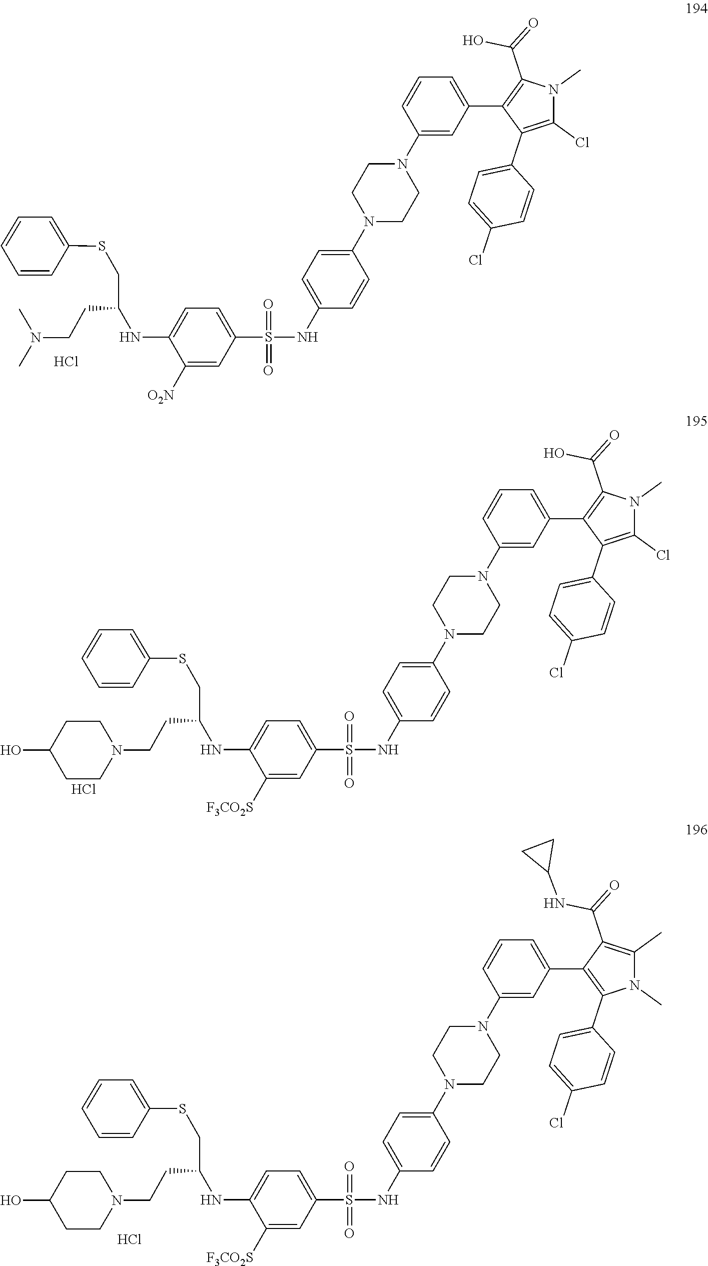

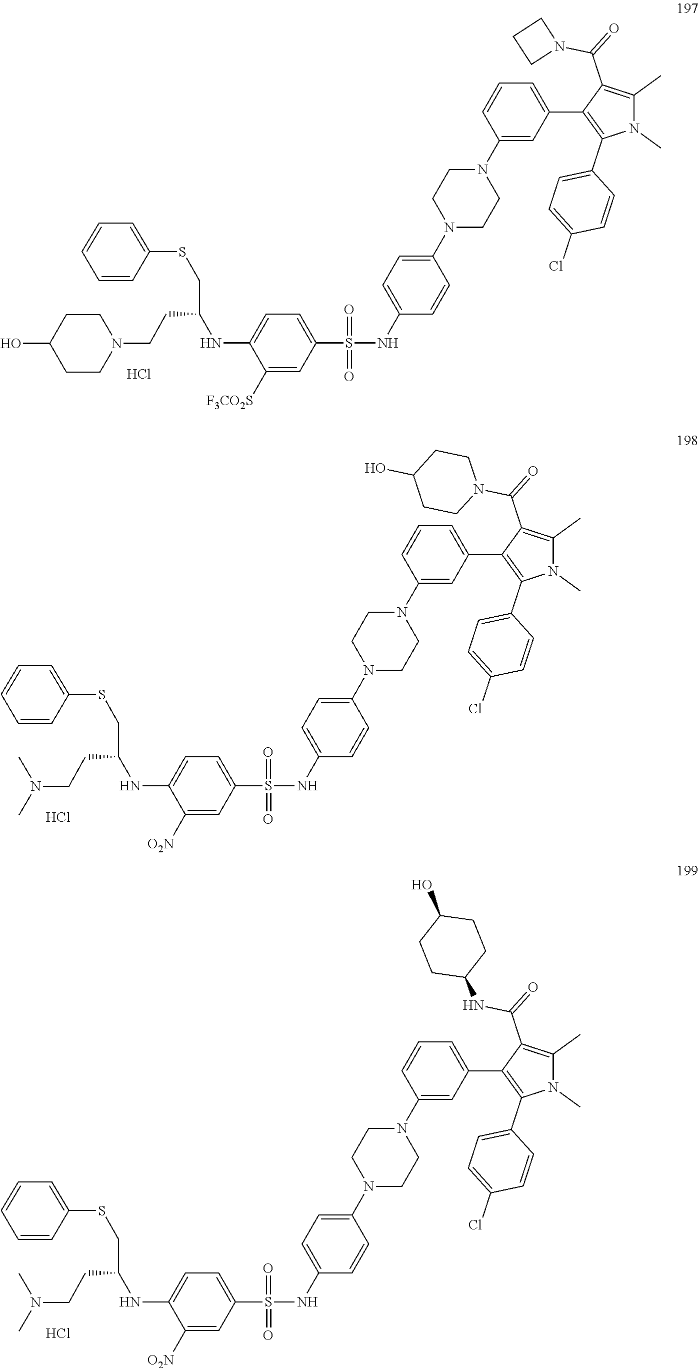

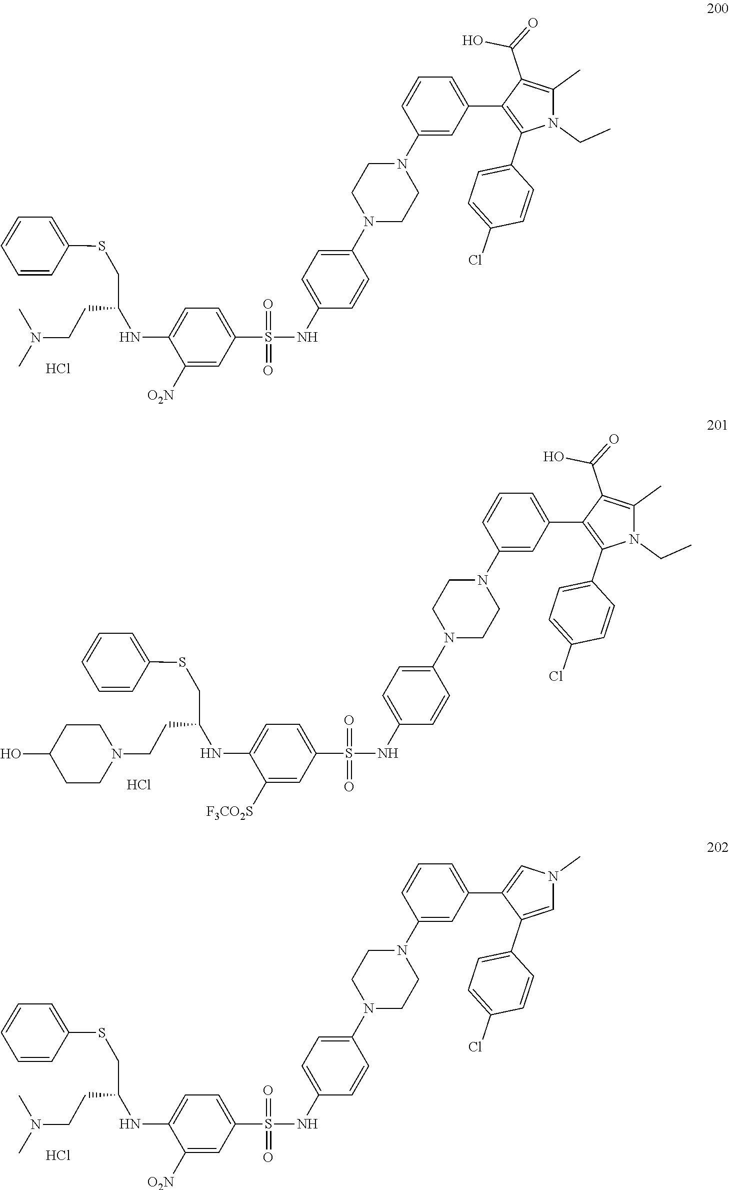

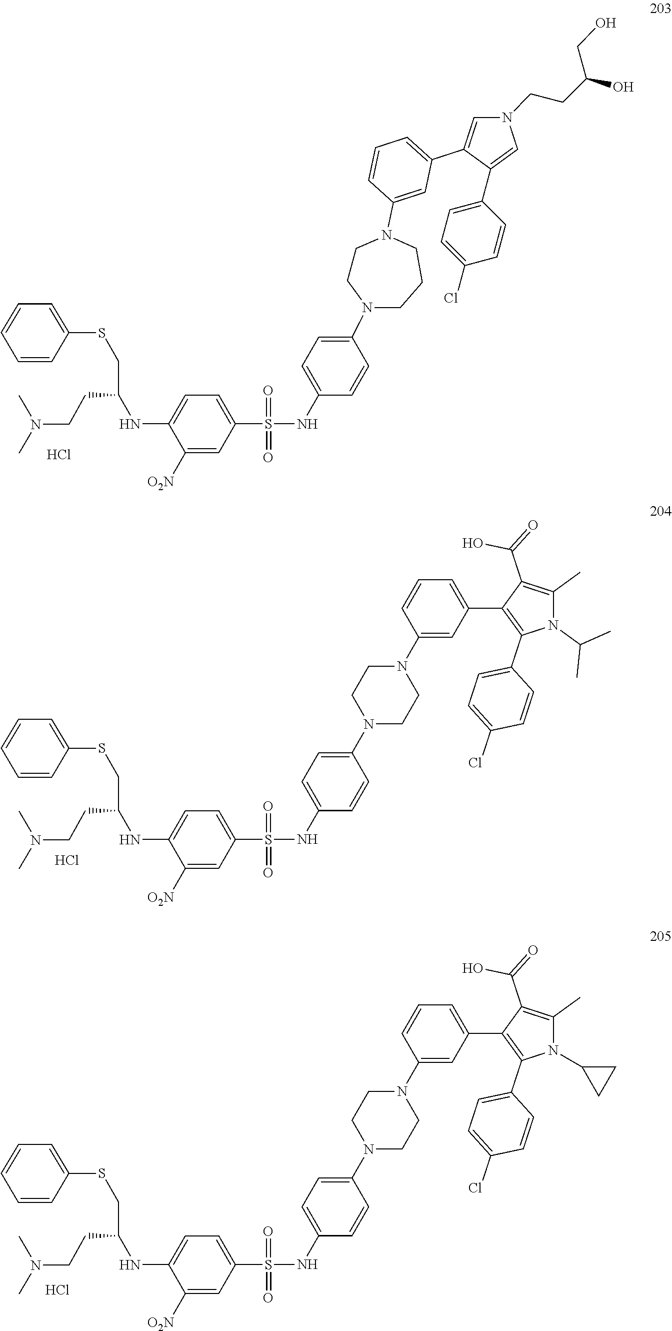 [SMILES: CN(C)CC[C@H](CSc1ccccc1)Nc1ccc(S(=O)(=O)Nc2ccc(N3CCCN(c4cccc(-c5cn(CC[C@H](O)CO)cc5-c5ccc(Cl)cc5)c4)CC3)cc2)cc1[N+](=O)[O-] ; Cl ; Cc1c(C(=O)O)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C(C)C ; Cl ; Cc1c(C(=O)O)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)n1C1CC1 ; Cl]

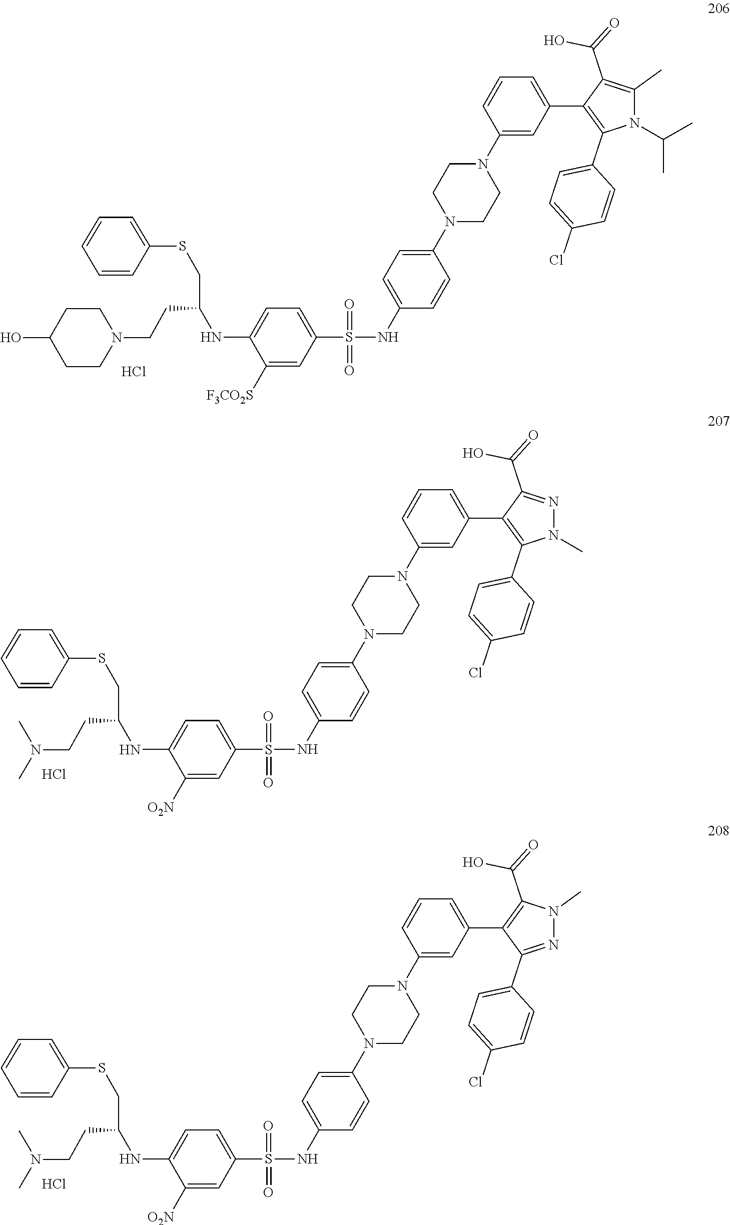

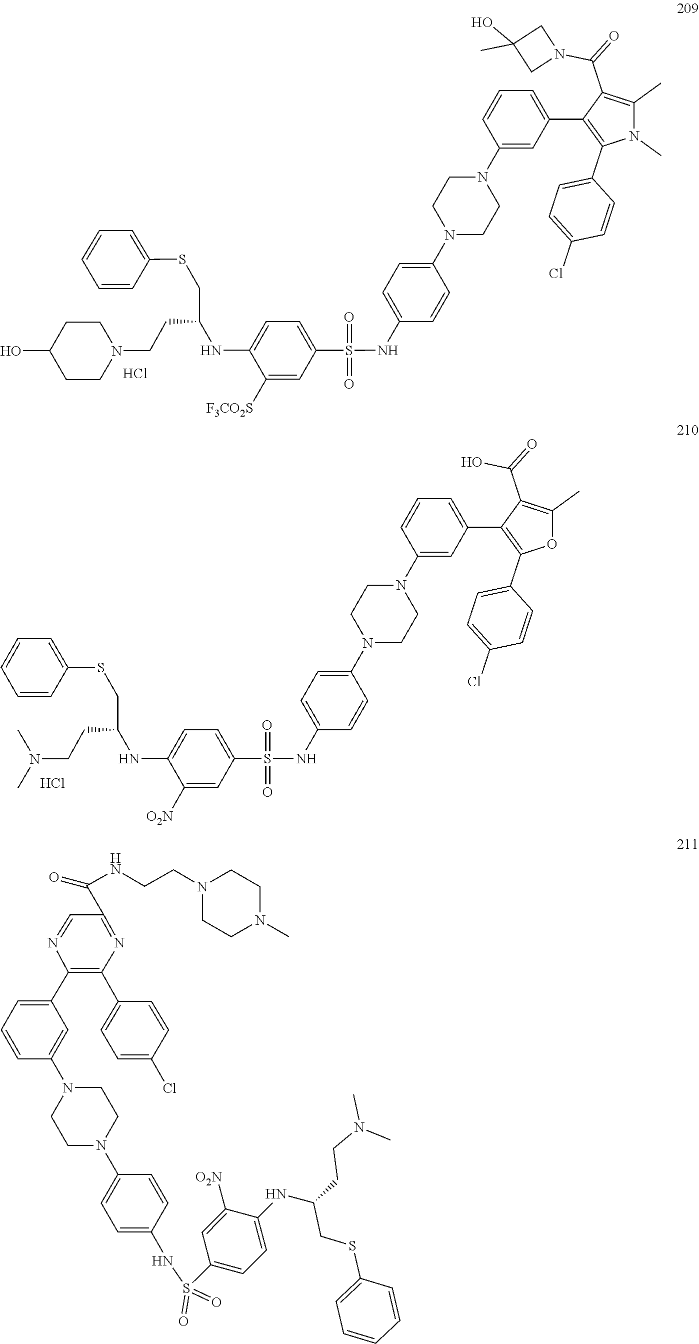

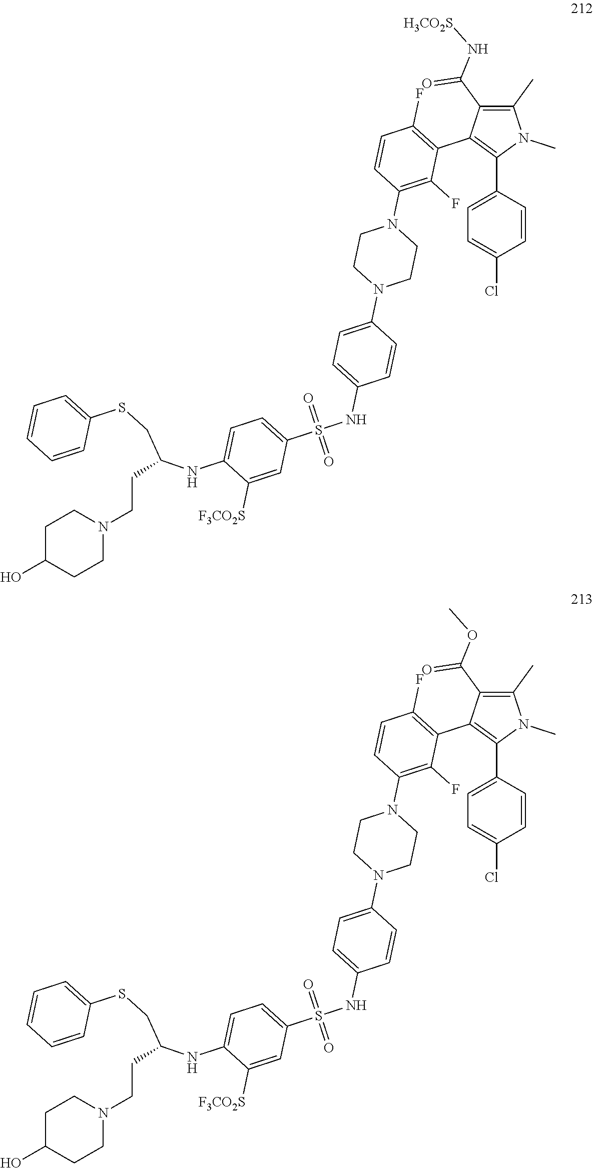

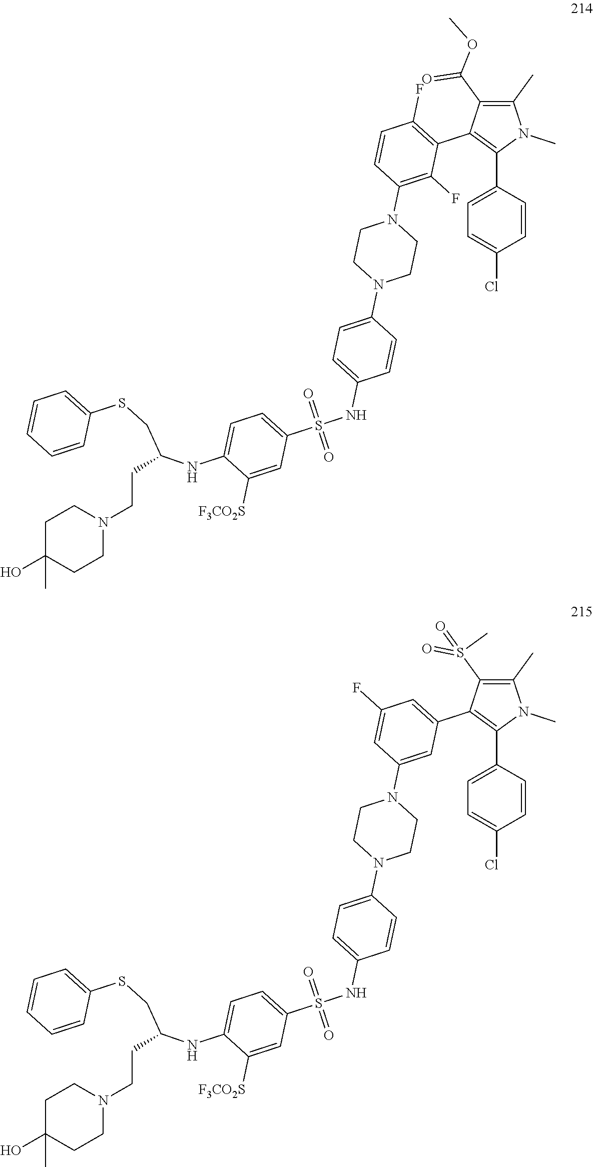

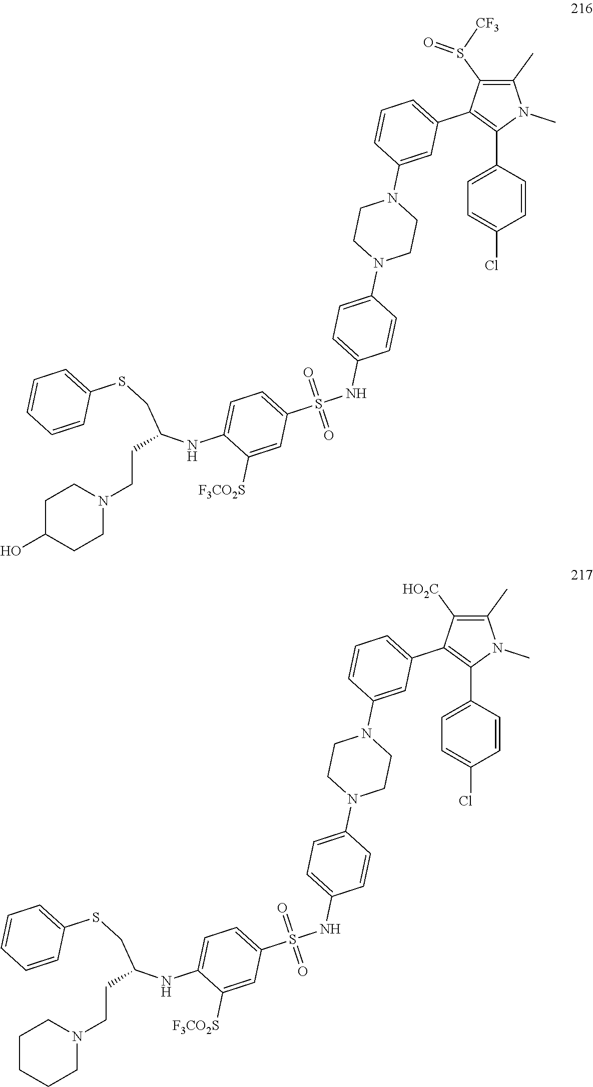

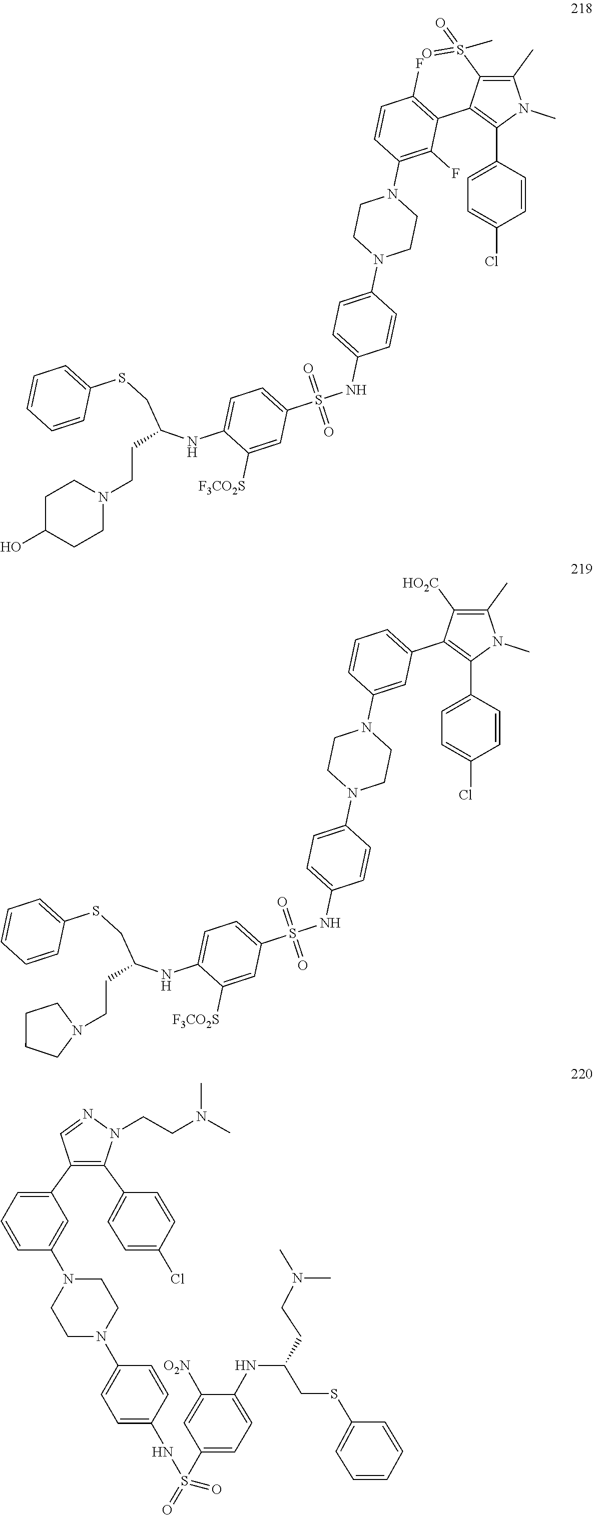

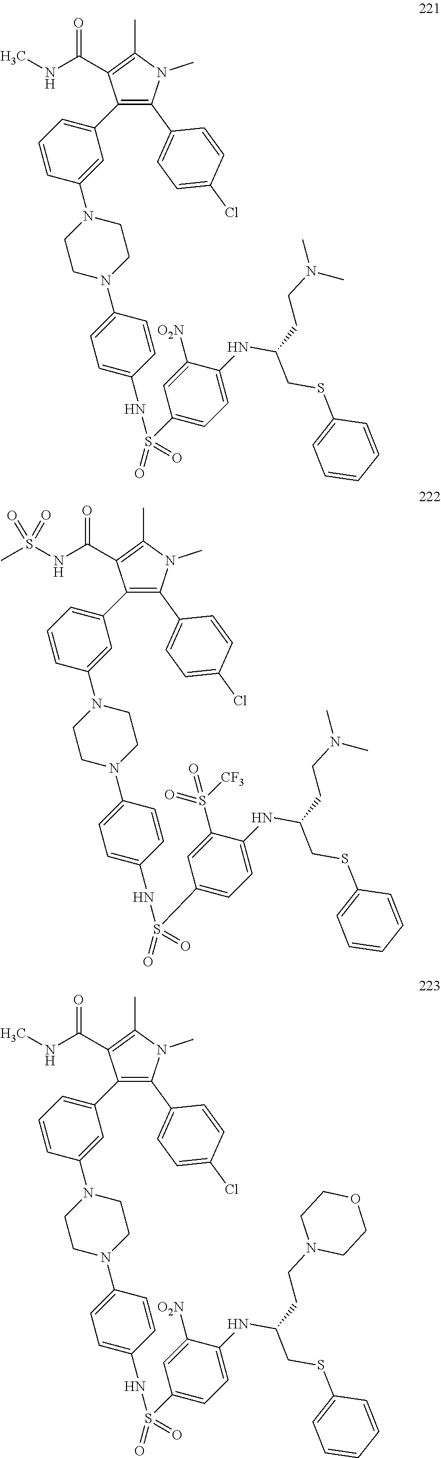

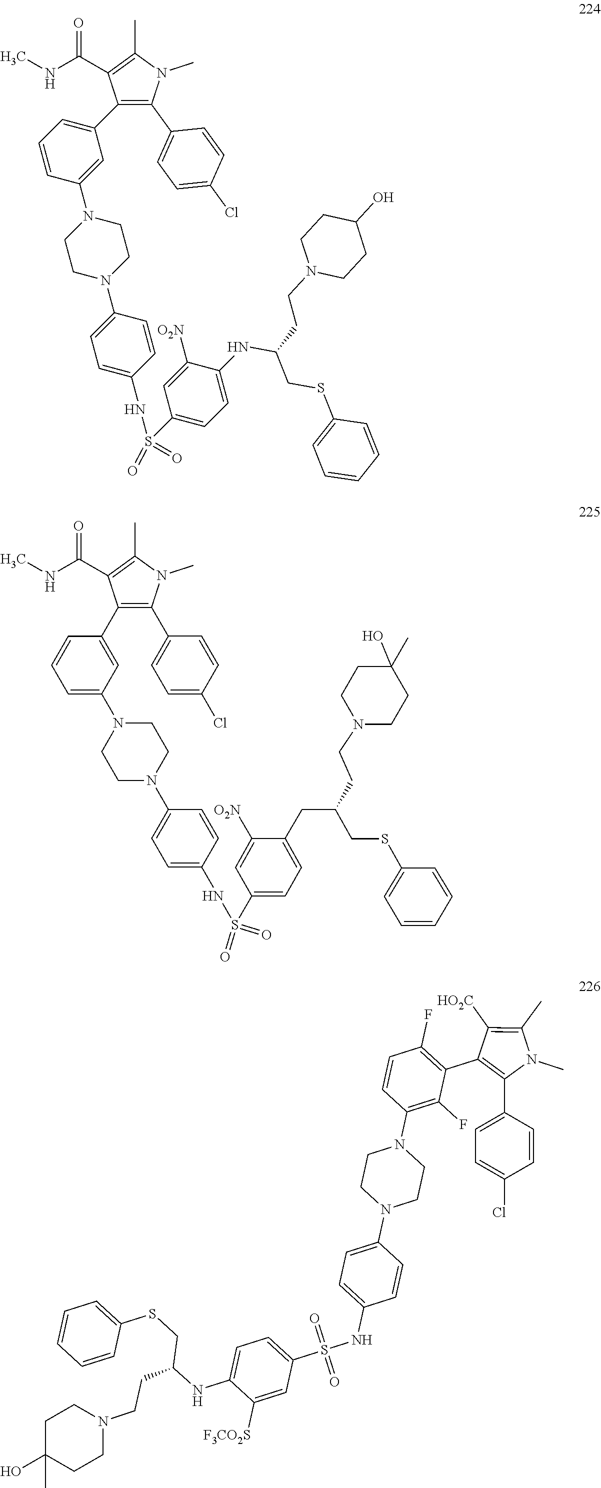

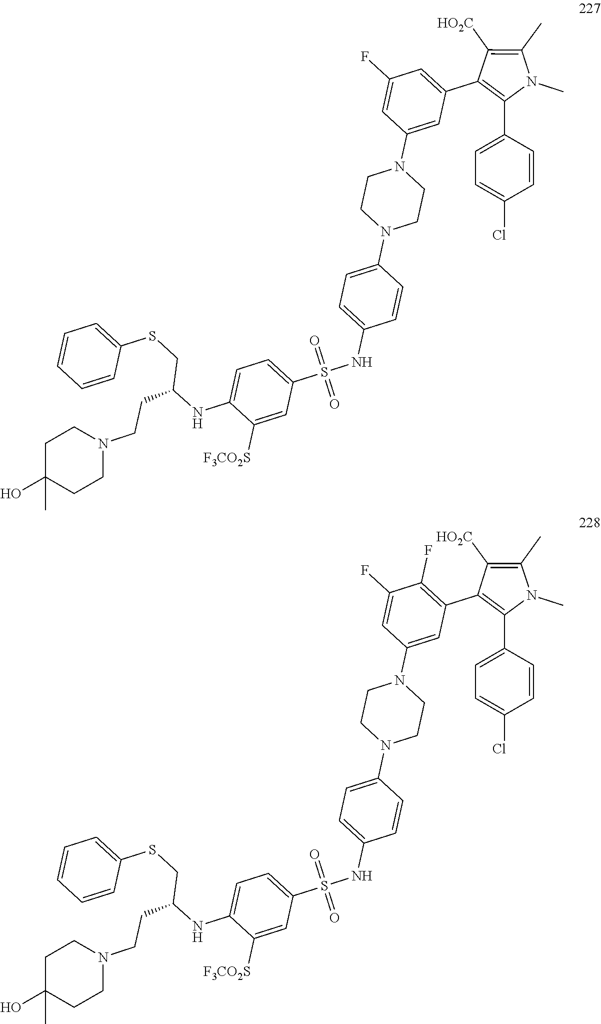

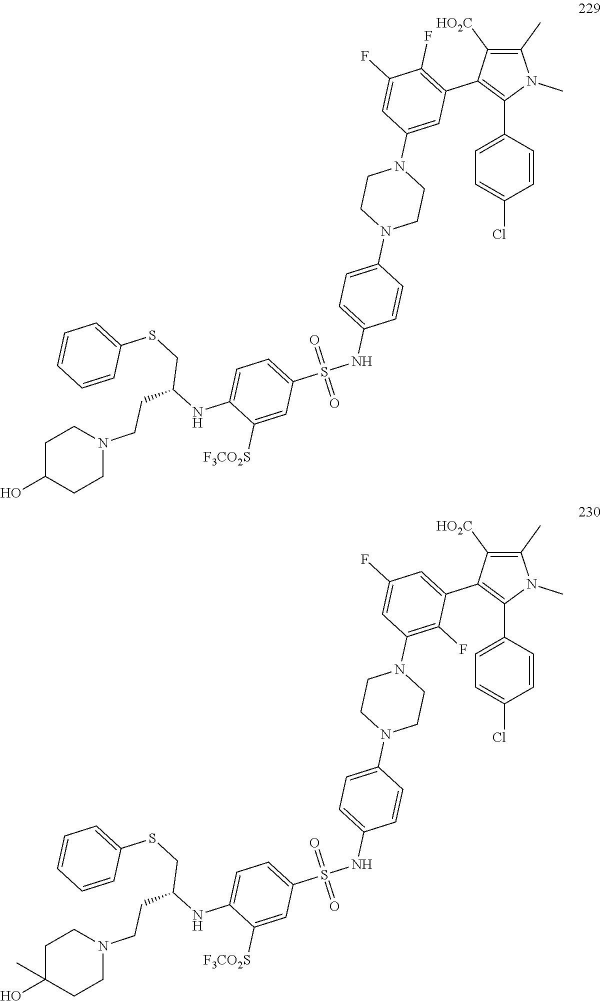

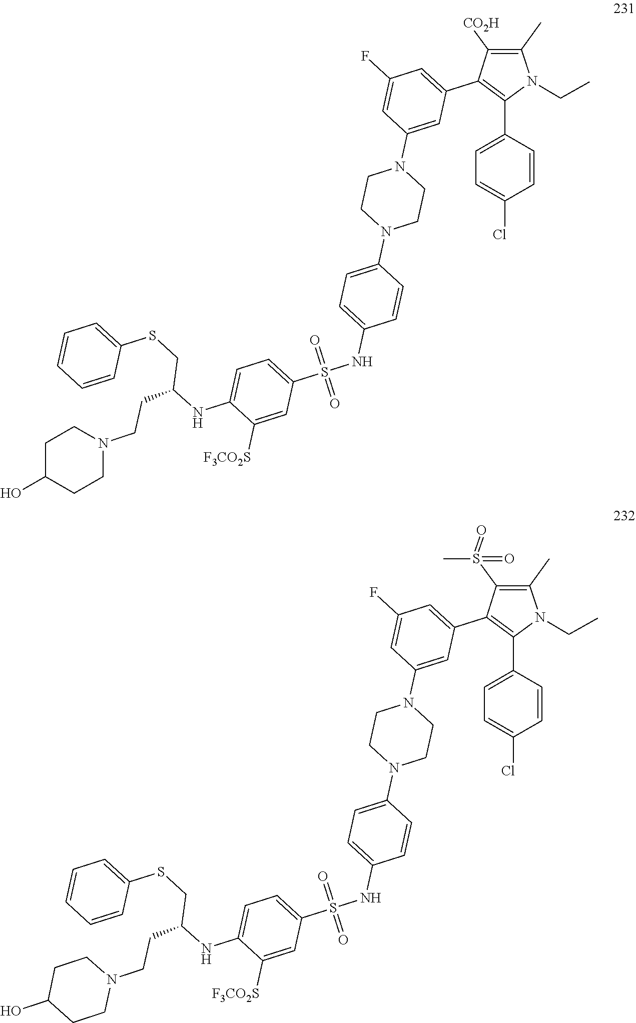

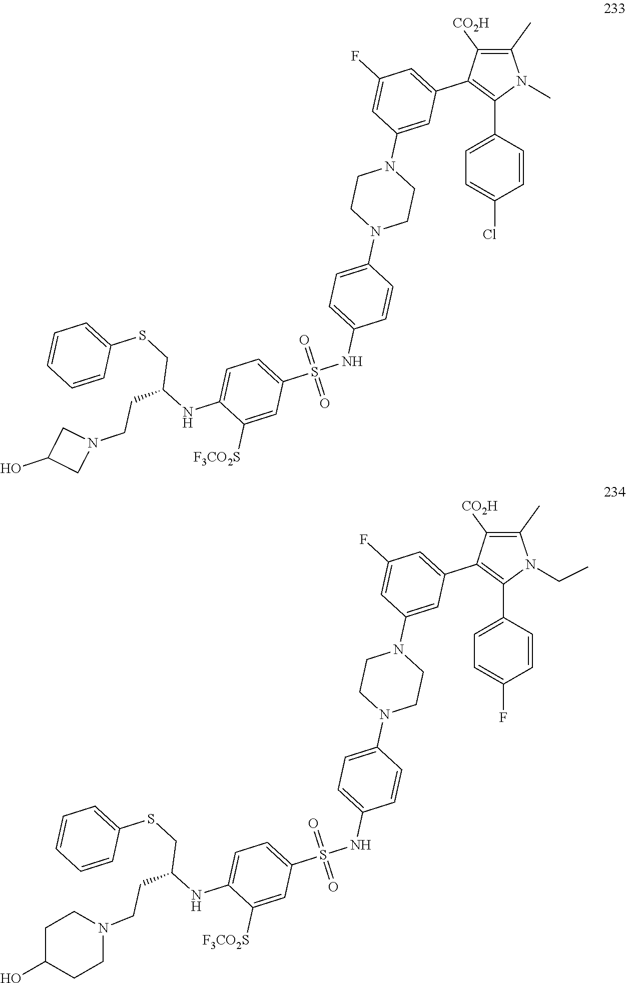

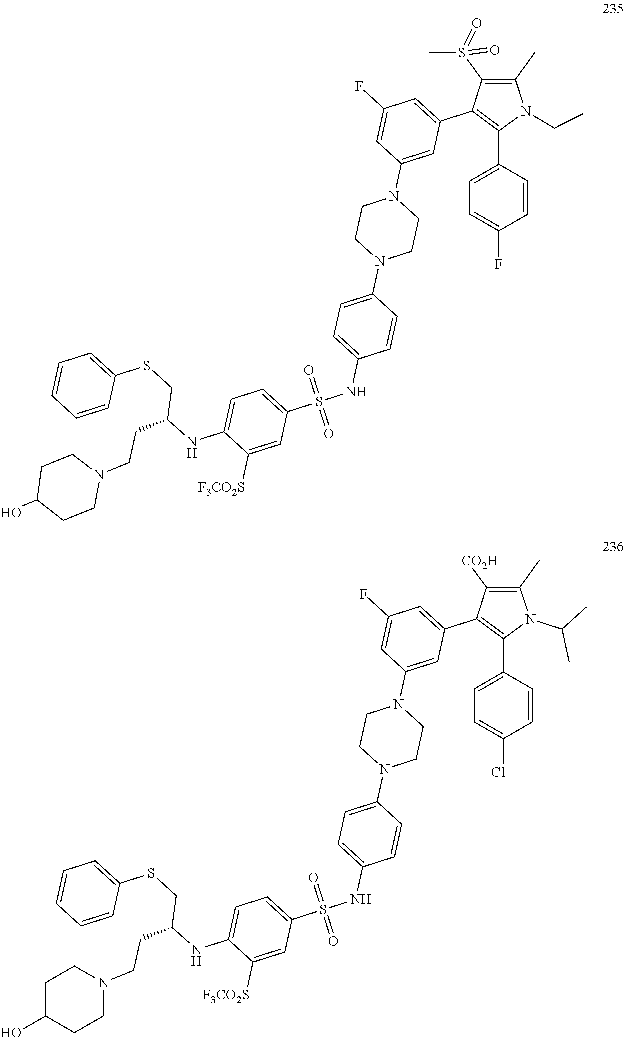

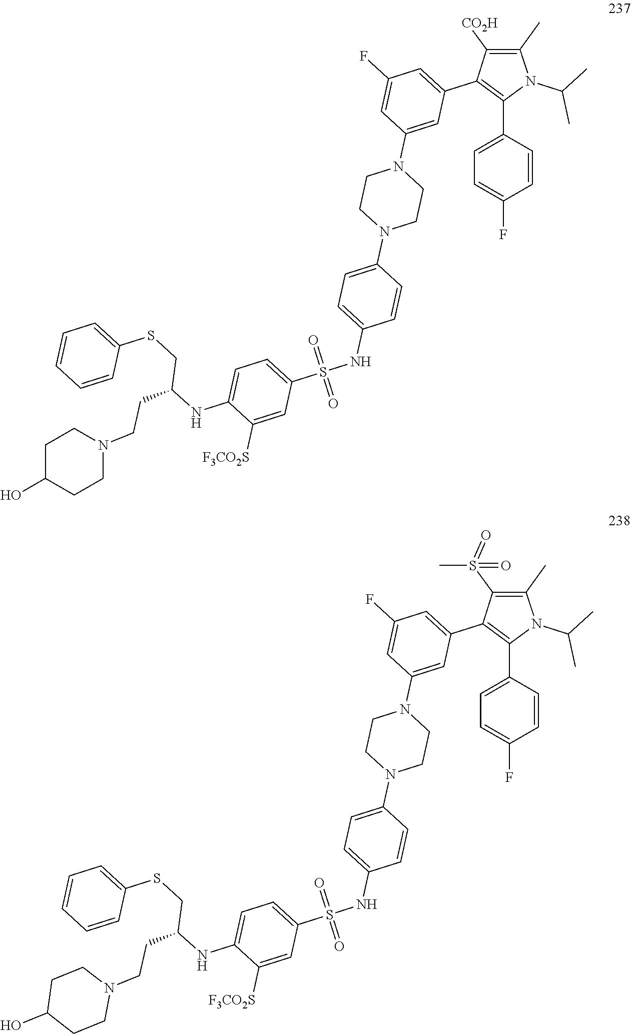

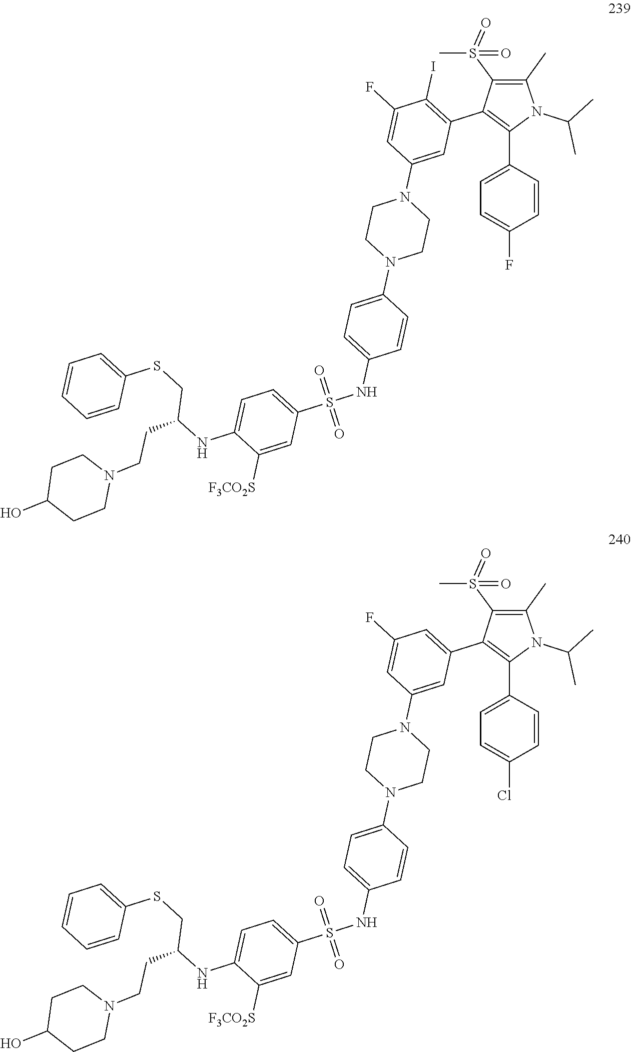

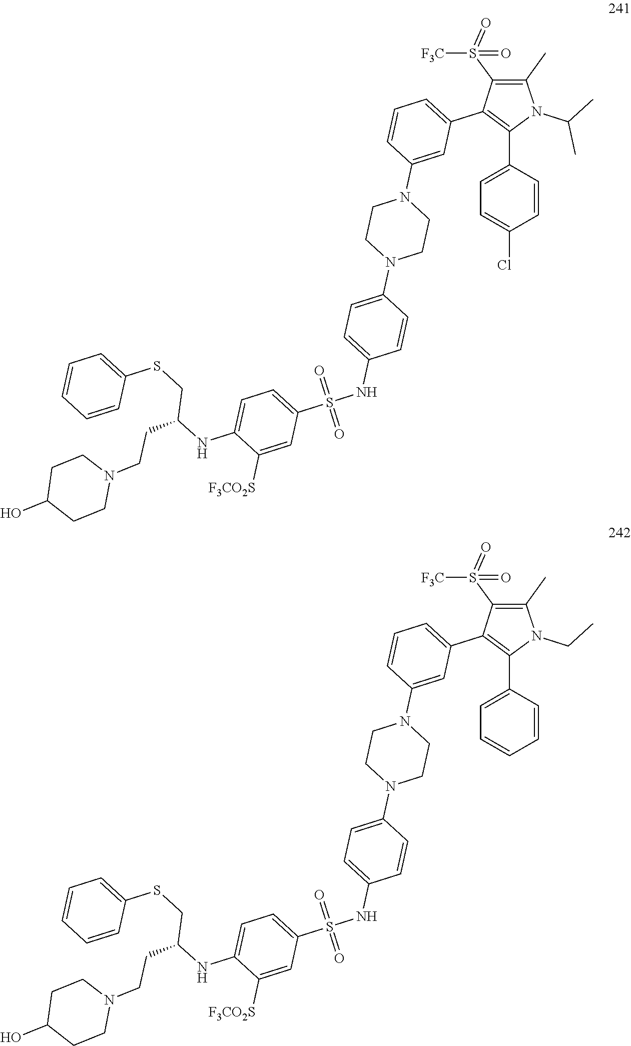

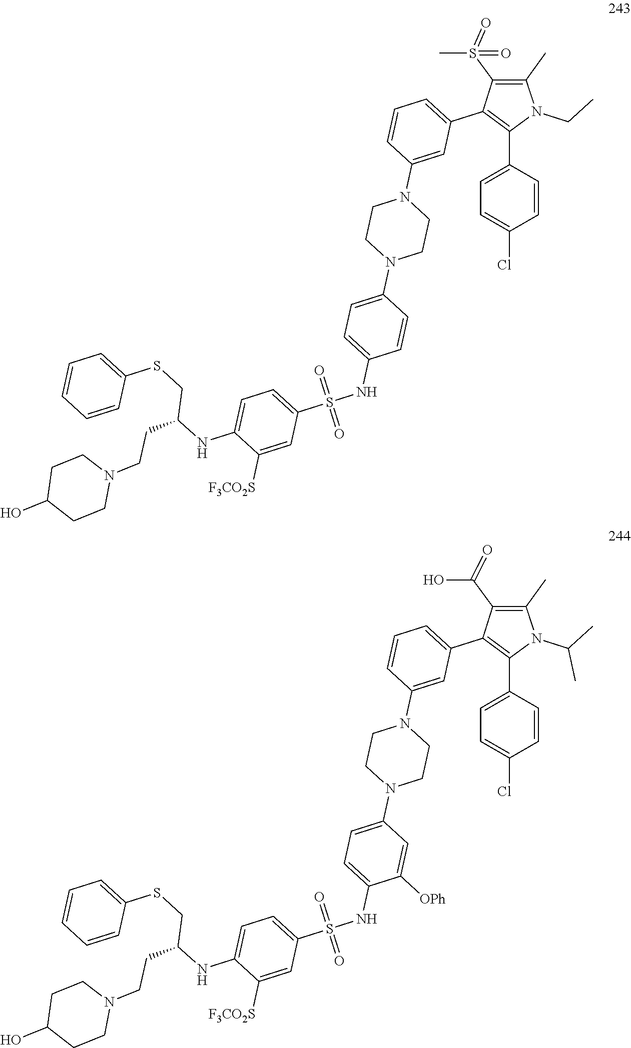

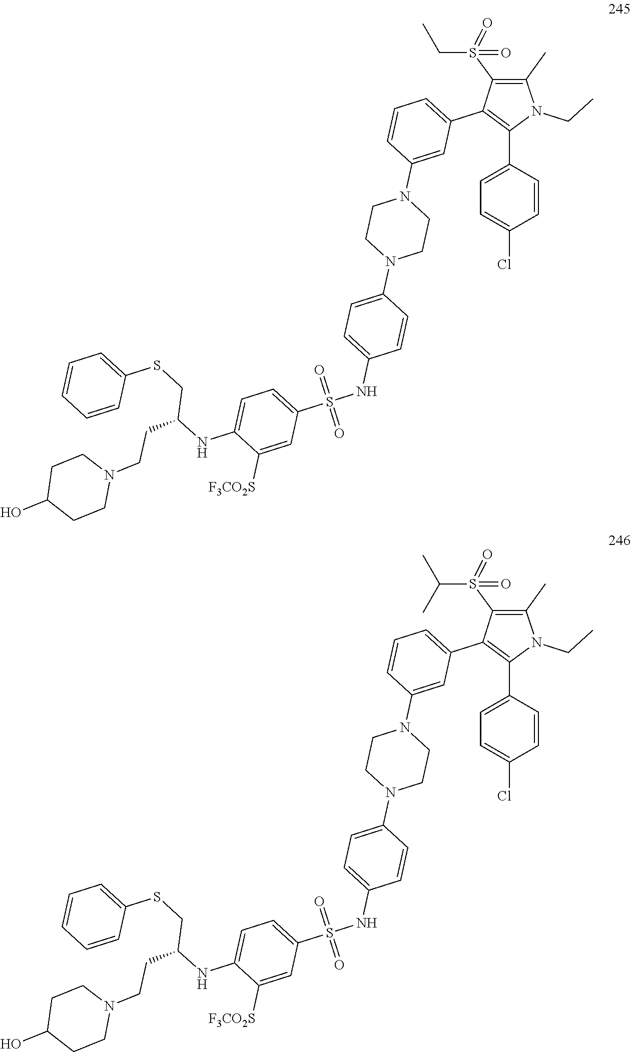

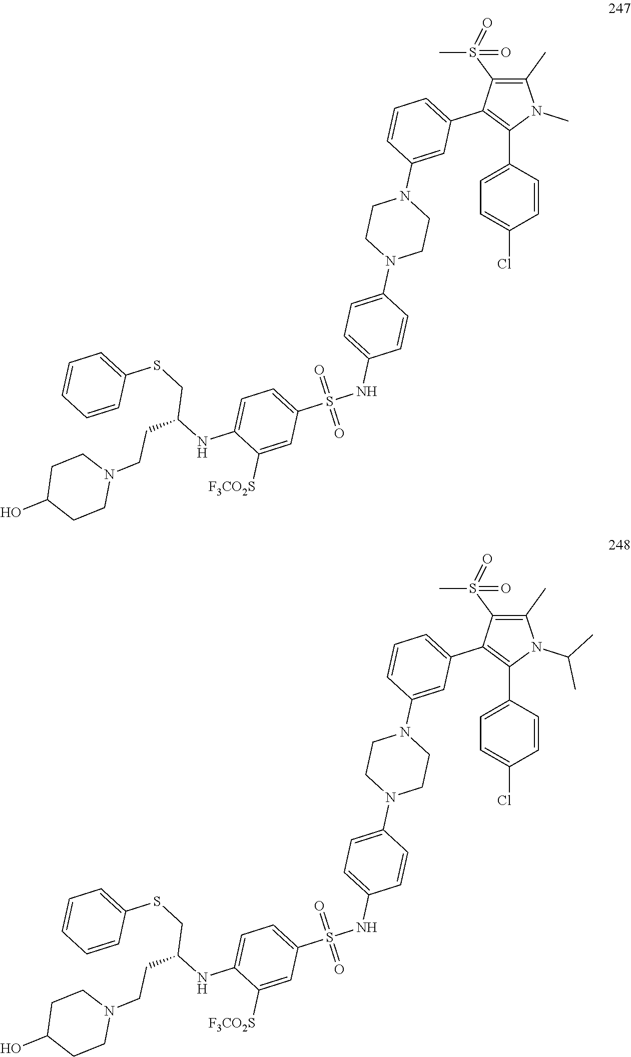

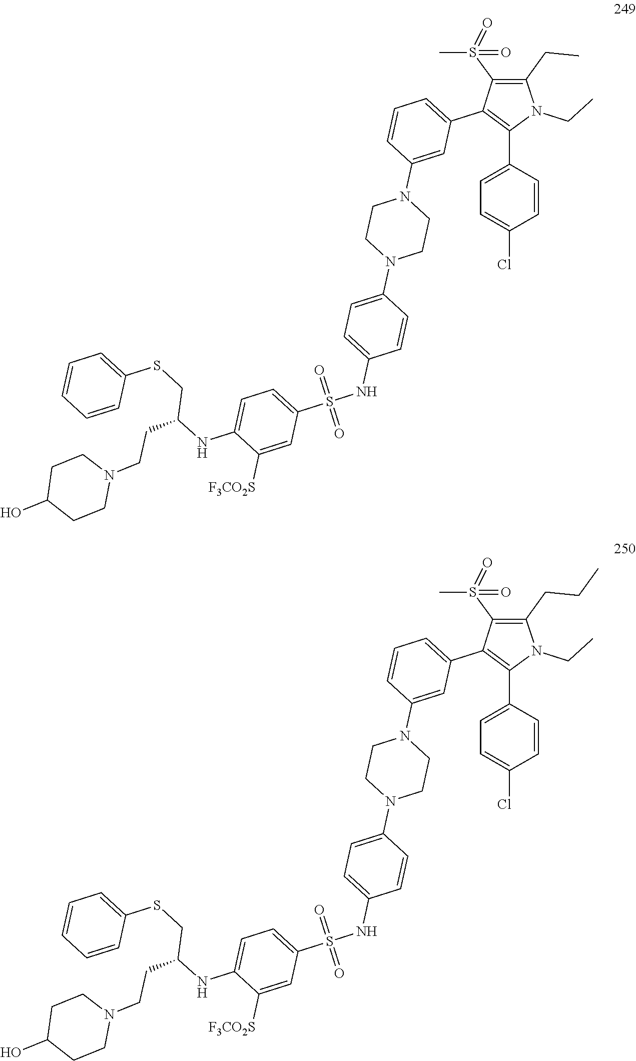

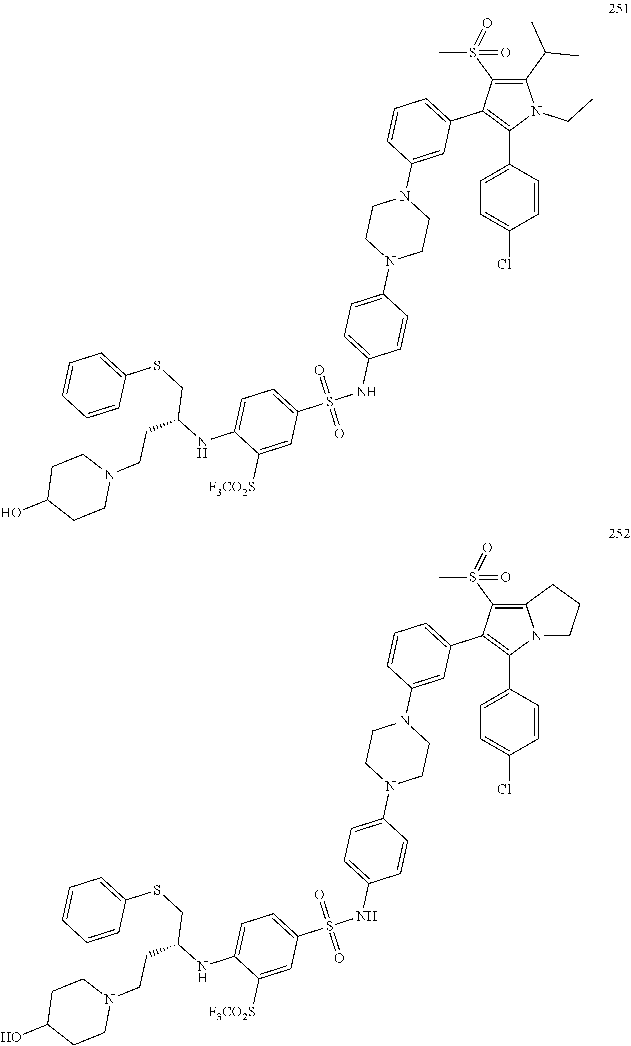

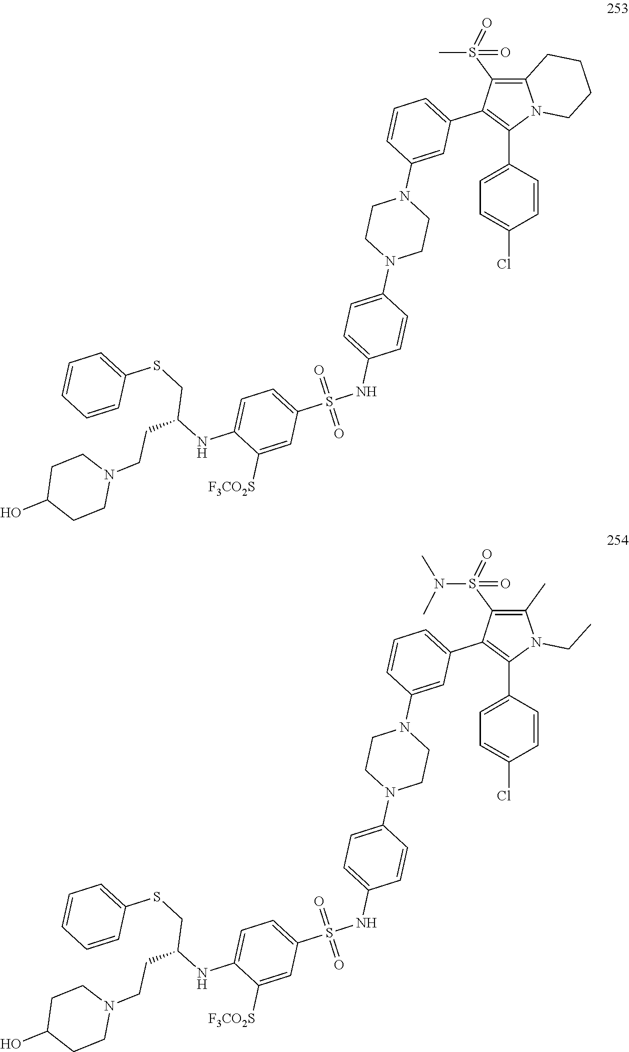

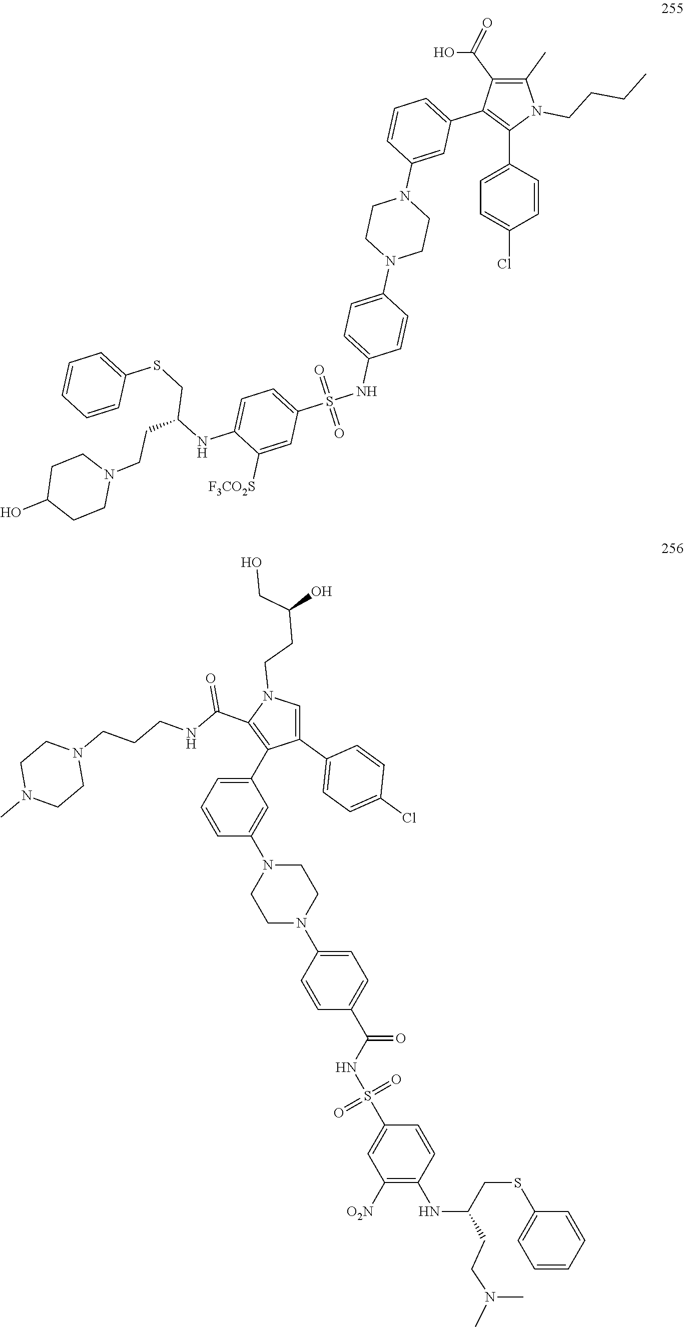

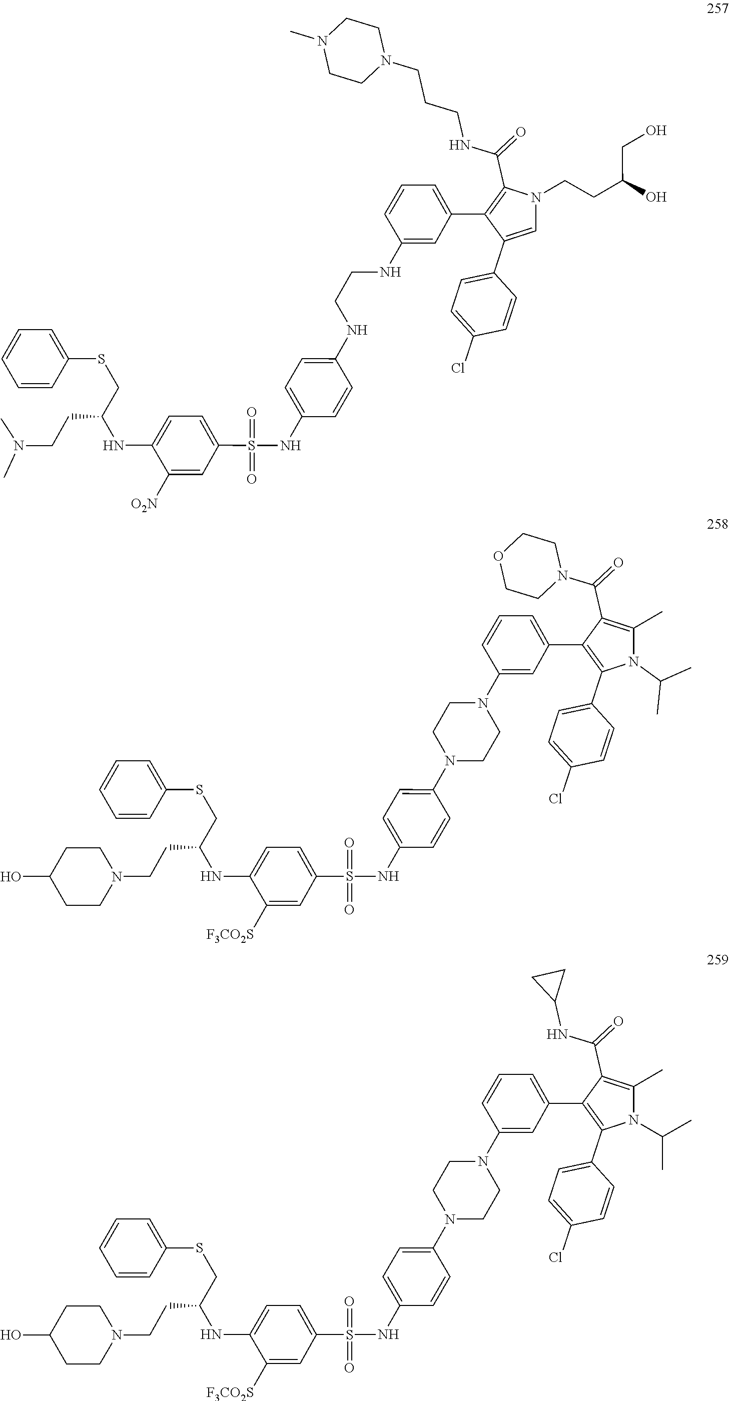

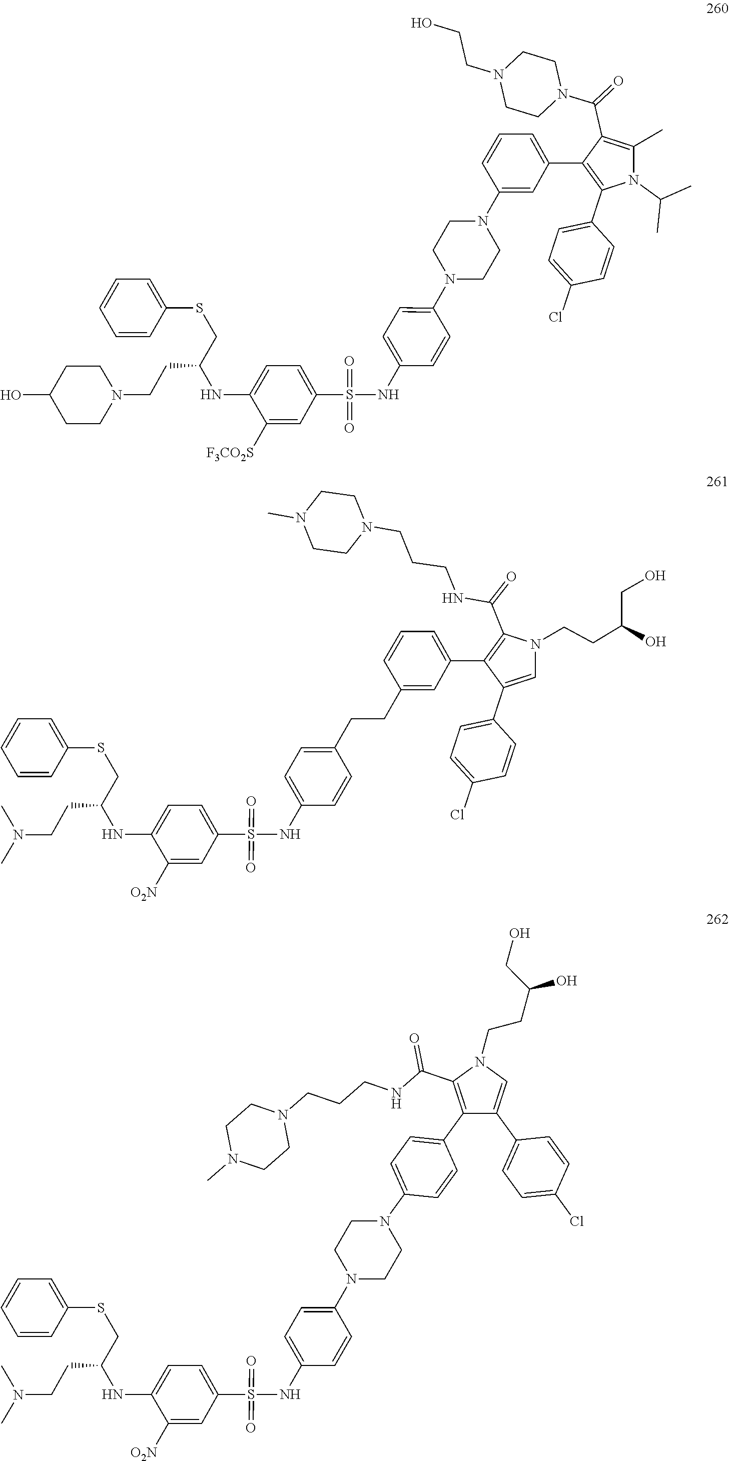

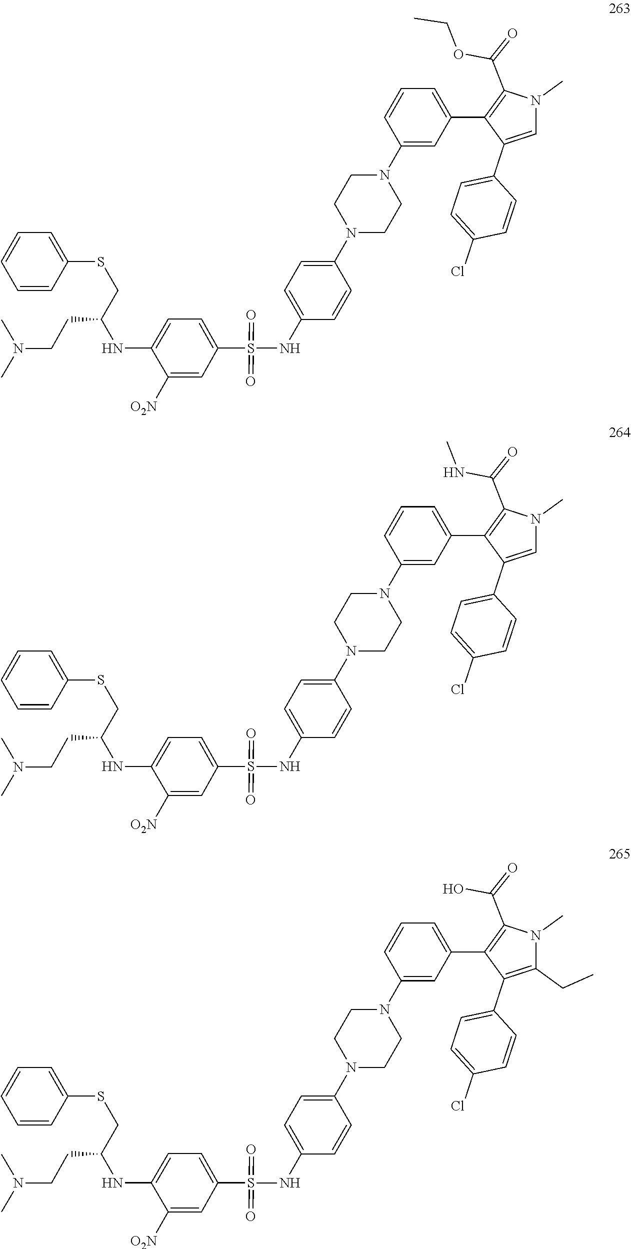 [SMILES: CCOC(=O)c1c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)cn1C ; CNC(=O)c1c(-c2cccc(N3CCN(c4ccc(NS(=O)(=O)c5ccc(N[C@H](CCN(C)C)CSc6ccccc6)c([N+](=O)[O-])c5)cc4)CC3)c2)c(-c2ccc(Cl)cc2)cn1C ; CCc1c(-c2ccc(Cl)cc2)c(-c2cccc(N3CCN(c4ccc(NS(=O)(=O)c5ccc(N[C@H](CCN(C)C)CSc6ccccc6)c([N+](=O)[O-])c5)cc4)CC3)c2)c(C(=O)O)n1C]

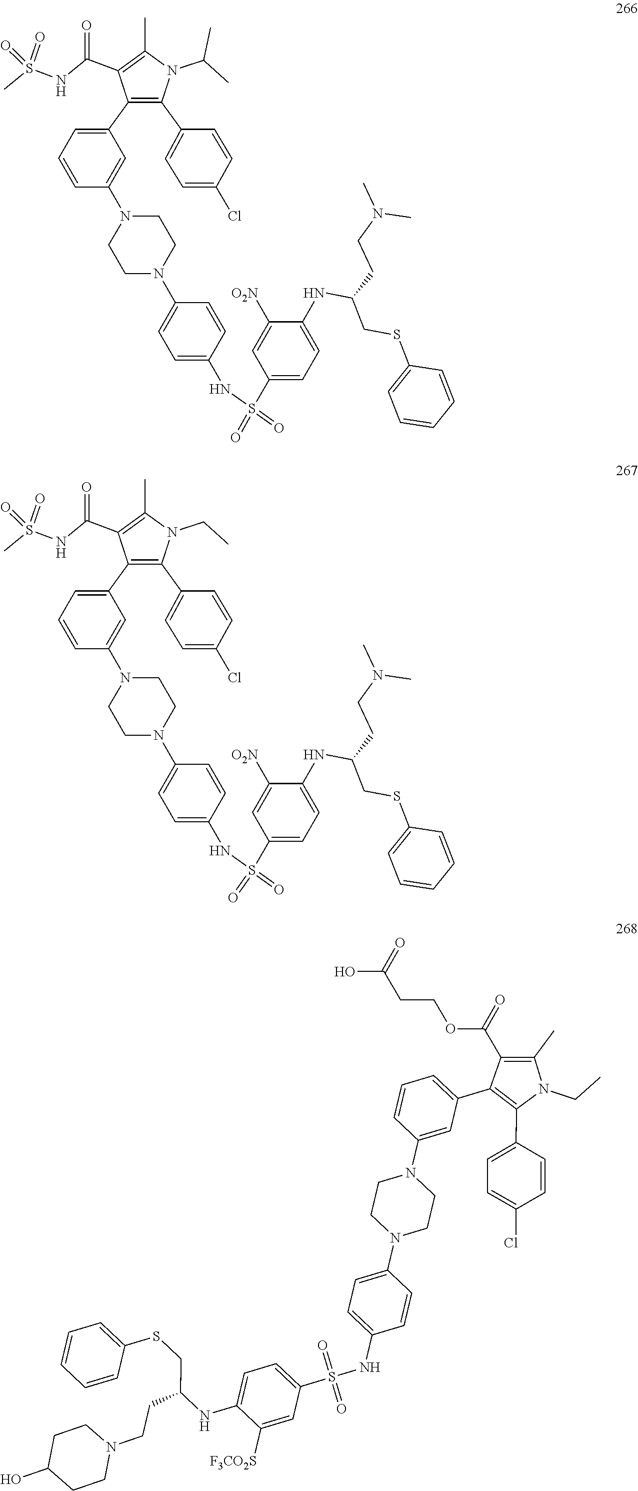

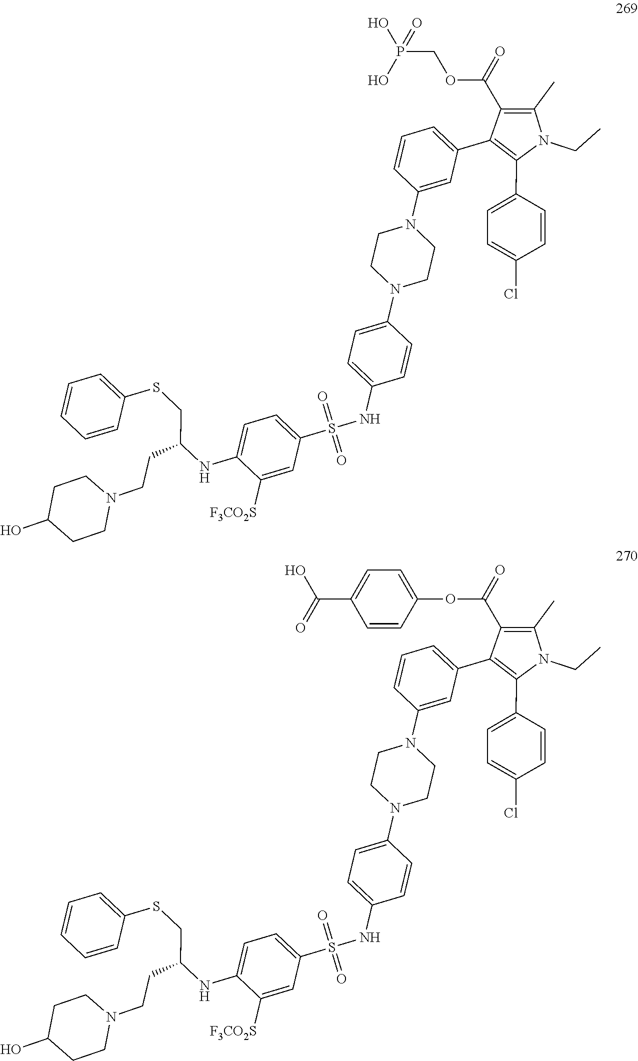

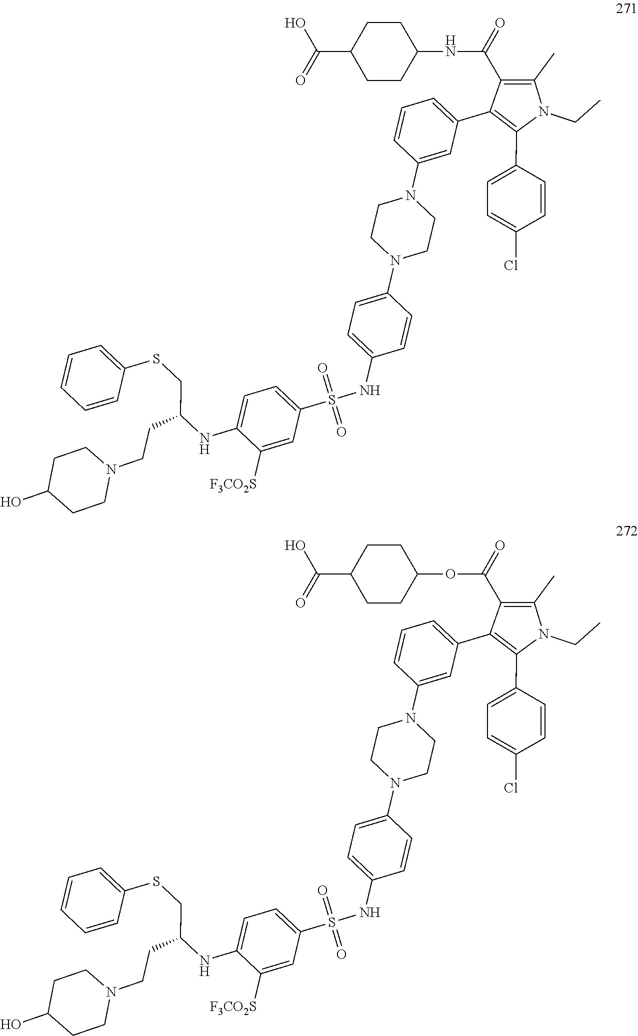

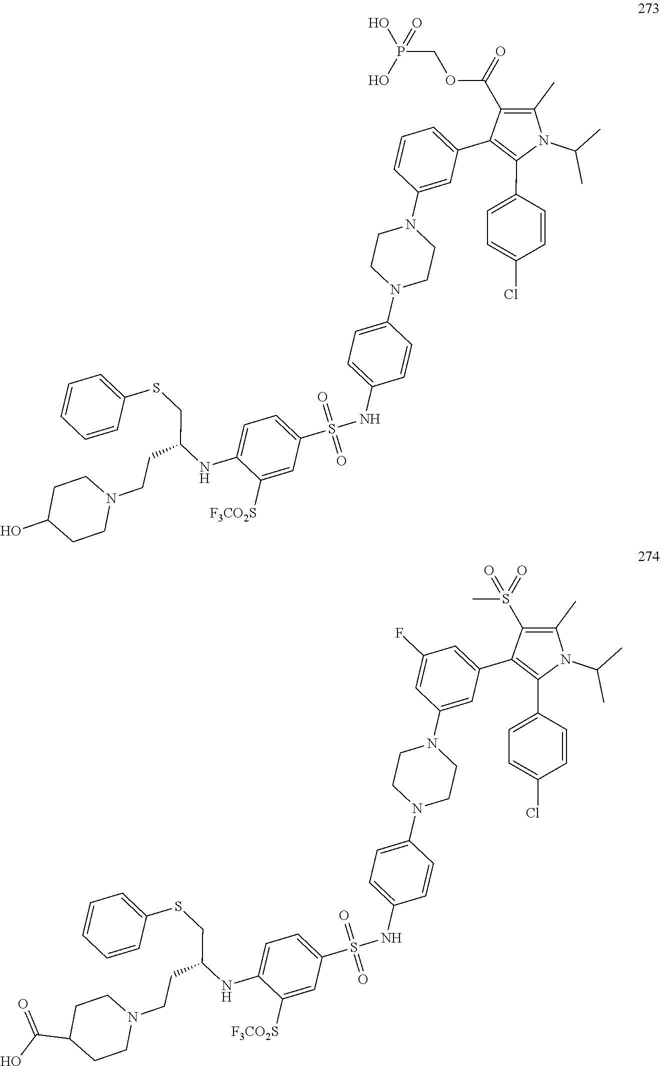

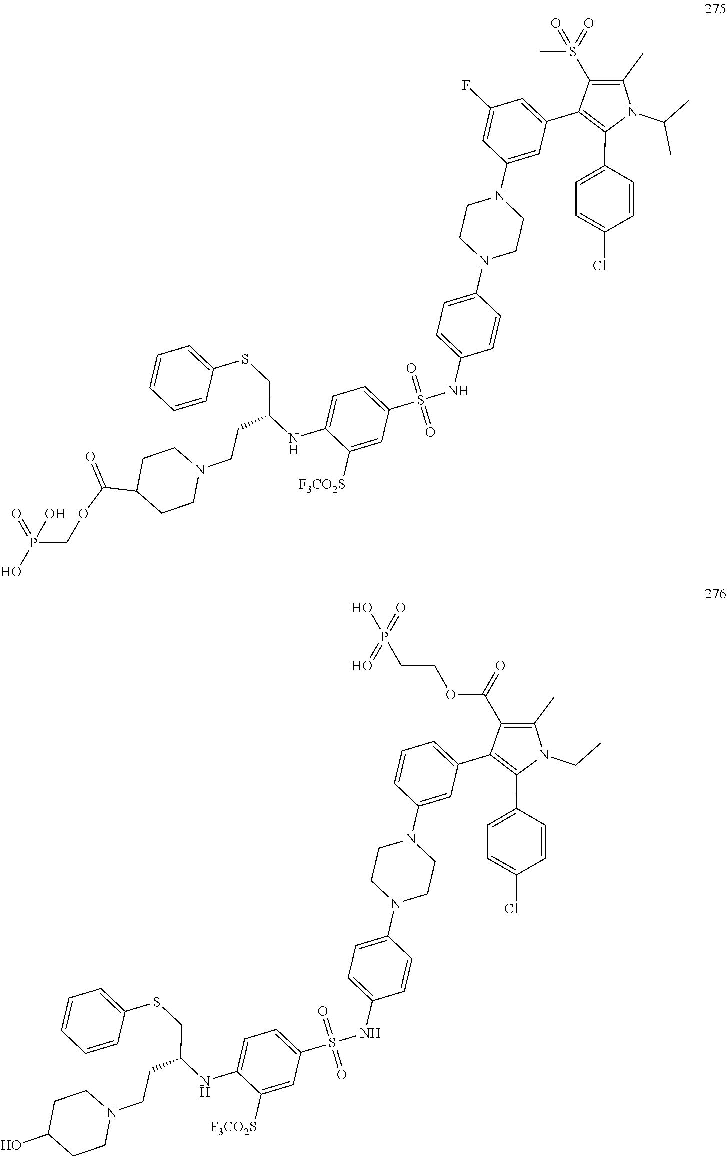

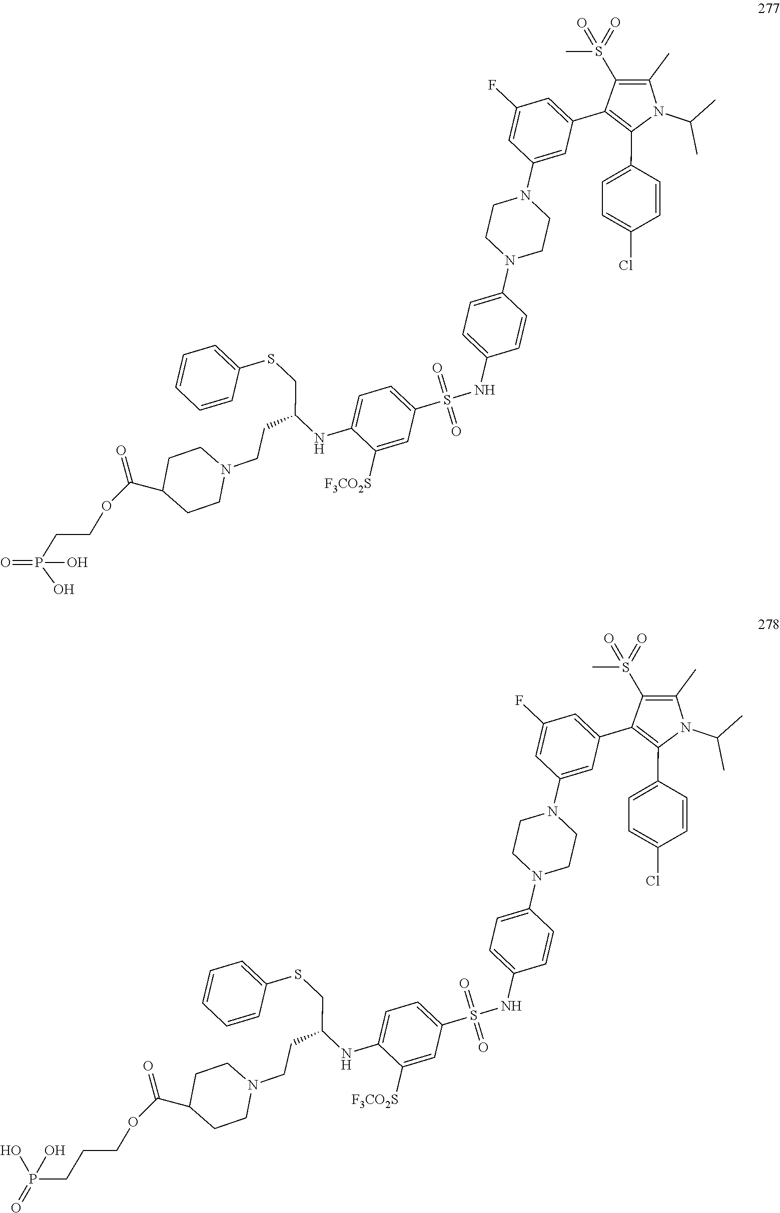

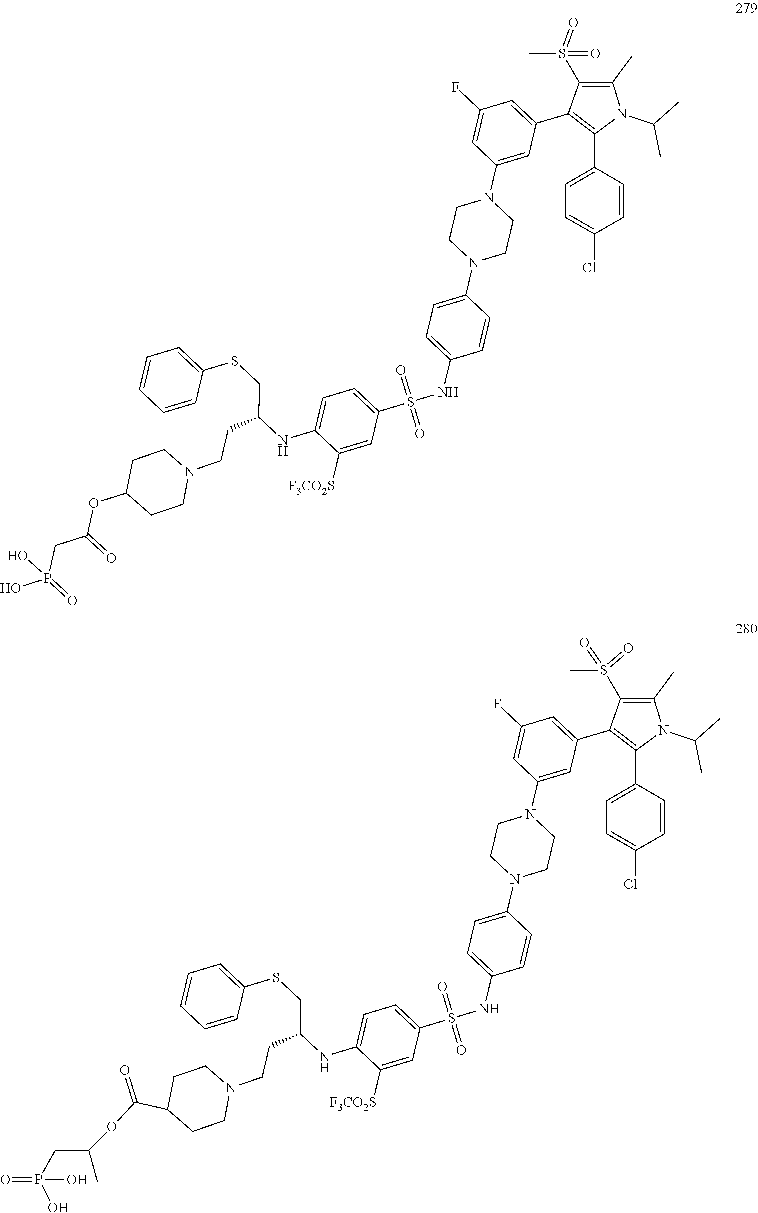

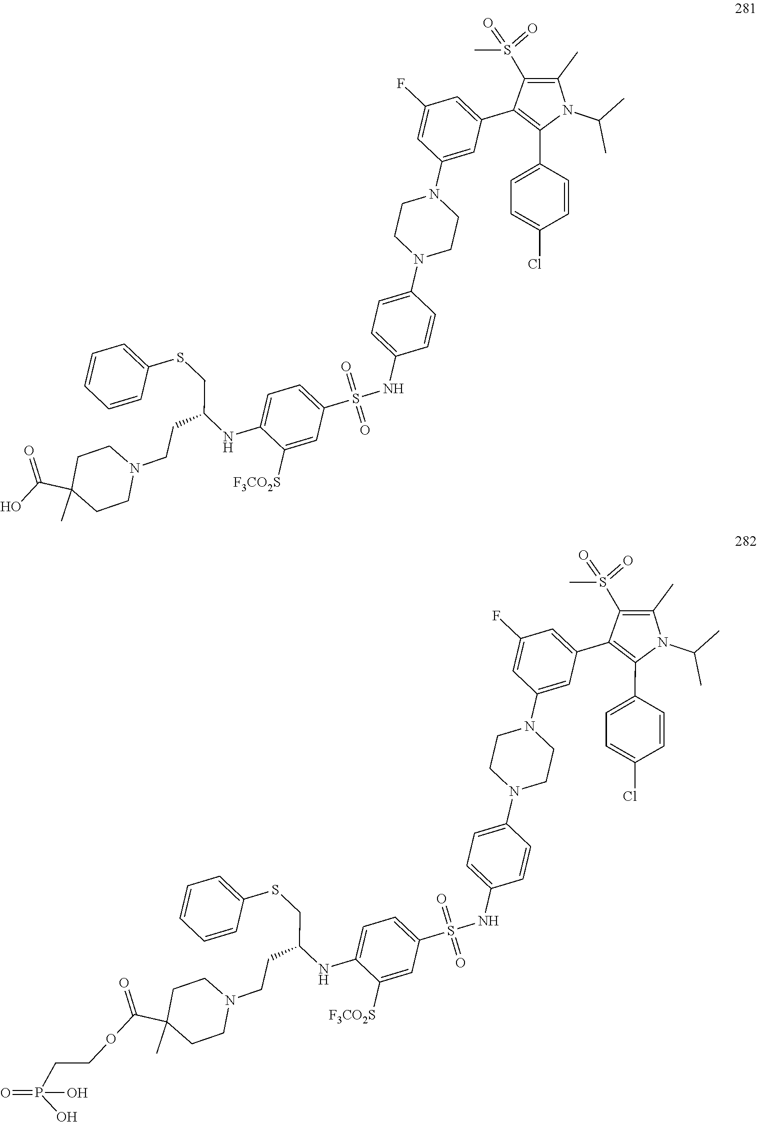

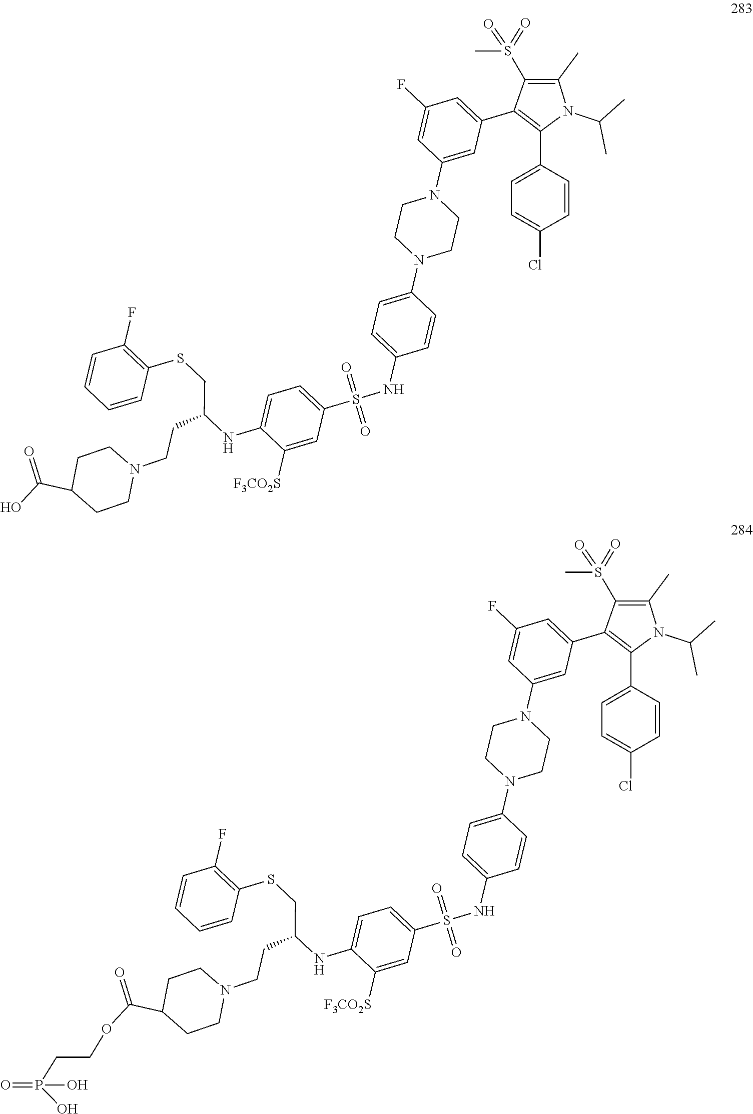

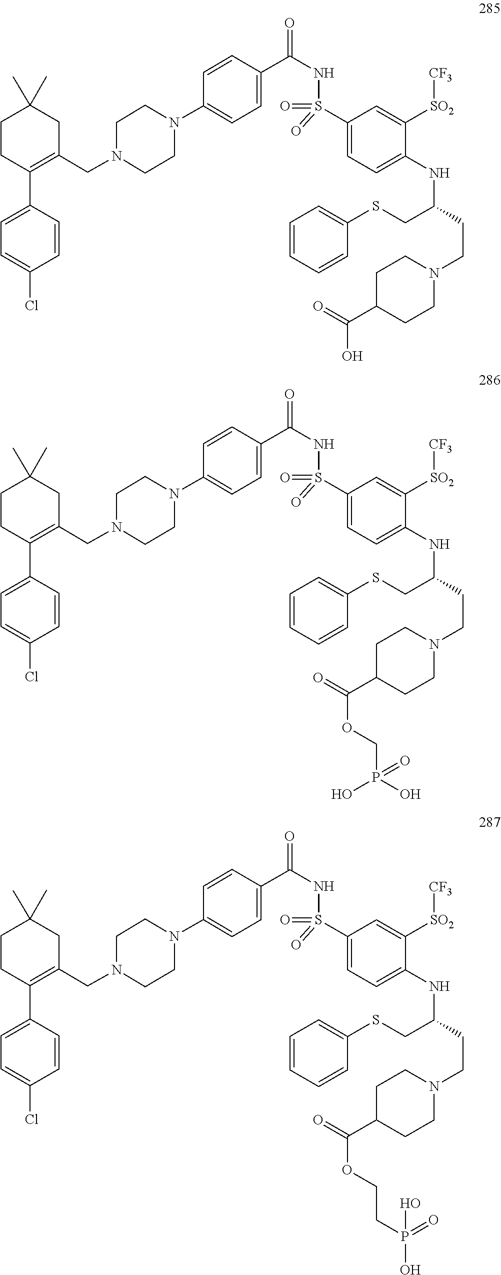 [SMILES: CC1(C)CCC(c2ccc(Cl)cc2)=C(CN2CCN(c3ccc(C(=O)NS(=O)(=O)c4ccc(N[C@H](CCN5CCC(C(=O)O)CC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCC(C(=O)OCP(=O)(O)O)CC5)CSc5ccccc5)c(S(=O)(=O)C(F)(F)F)c4)cc3)CC2)C1 ; CC1(C)CCC(c2ccc(Cl)cc2)=C(CN2CCN(c3ccc(C(=O)NS(=O)(=O)c4ccc(N[C@H](CCN5CCC(C(=O)OCCP(=O)(O)O)CC5)CSc5ccccc5)c(S(=O)(=O)C(F)(F)F)c4)cc3)CC2)C1]

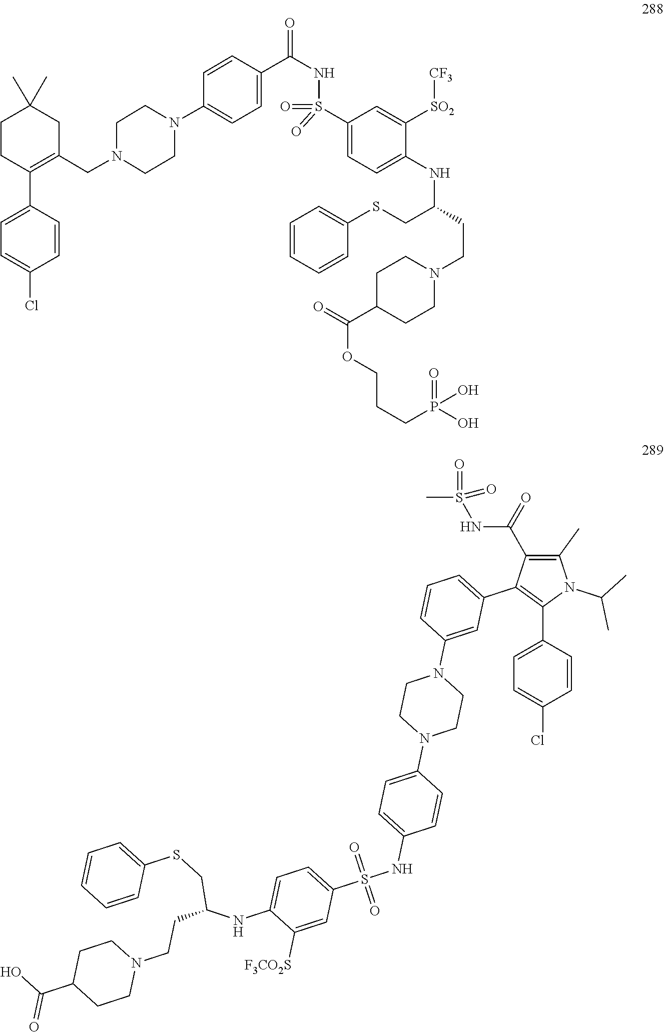

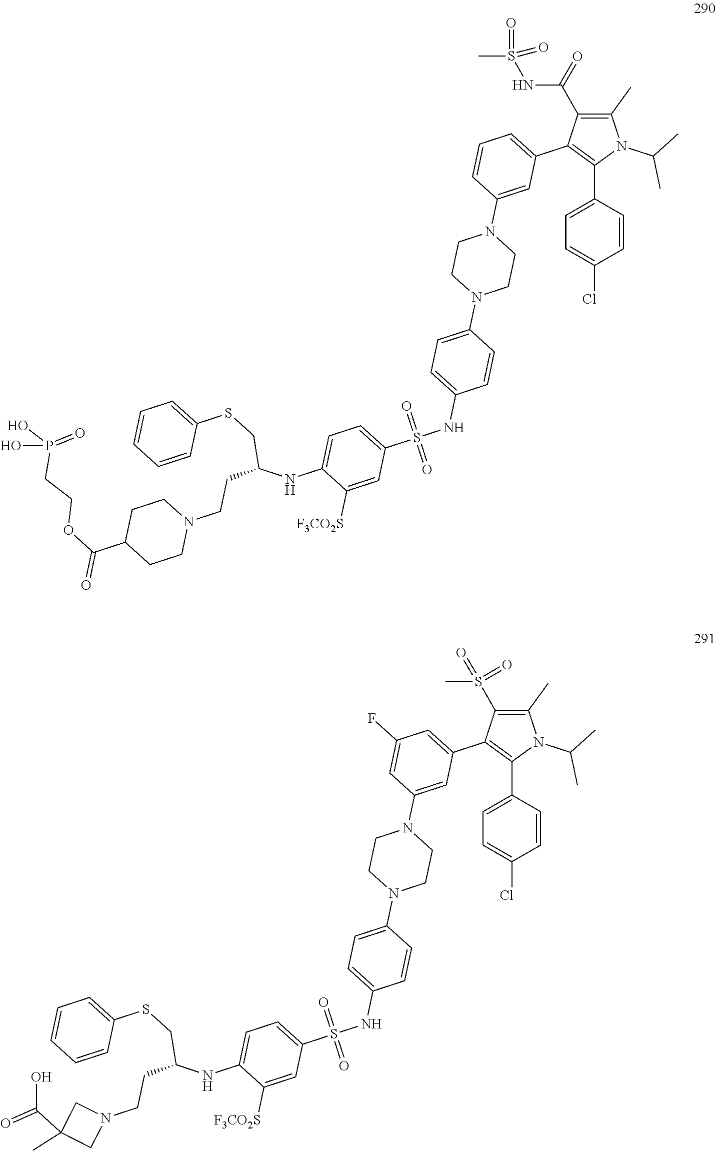

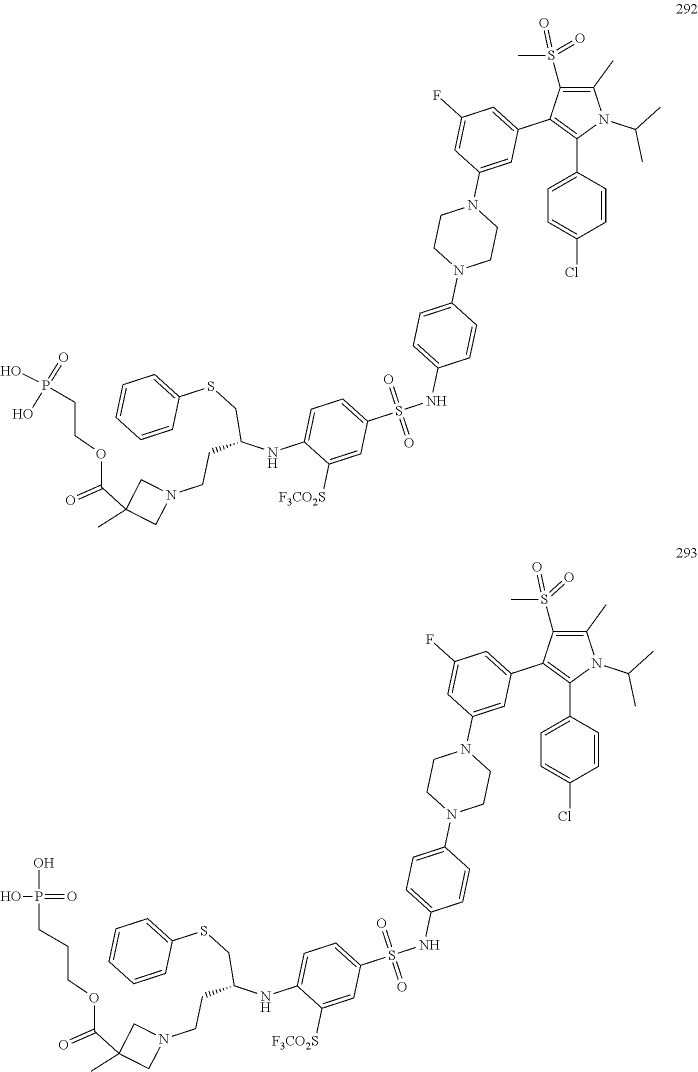

### Purity of Compounds of the Invention.

Any compound herein can be purified. In some embodiments, a compound, complex, ligand, or peptide herein is at least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically-Acceptable Salts.

The invention provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Optional Substituents for Chemical Groups.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### Pharmaceutical Formulations.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by any form and route known in the art including, for example, intravenous, subcutaneous, intramuscular, oral, rectal, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically acceptable carriers or excipients. Such carriers can be used to formulate liquids, gels, syrups, elixirs, slurries, or suspensions for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, is first melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising compounds described herein can be manufactured in a conventional manner, for example, by means of conventional mixing, dissolving, granulating, or emulsifying.

The pharmaceutical compositions can include at least one pharmaceutically acceptable carrier, diluent, or excipient and compounds described herein or pharmaceutically-acceptable salt form.

Methods for the preparation of compositions comprising the compounds described herein can include formulating the compounds with one or more inert, pharmaceutically-acceptable excipients. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine) A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is present in a composition in an amount ranging from about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

The foregoing ranges are merely suggestive. Dosages can be altered depending on a number of variables, including, for example, the activity of the compound used, the disease or condition to be treated, the mode of administration, the requirements of the individual subject, the severity of the disease or condition being treated, and the judgment of the practitioner.

In some embodiments, are methods for treating diseases or disorders comprising administering a compound described herein to a subject in need thereof. In some embodiments, the compound is administered in a manner that would be considered ineffective for treating any condition herein. In some embodiments, the compound is administered in a decreased cumulative dose, over multiple therapeutic cycles compared with the amount required for cancer therapy.

In some embodiments, the compound is administered within a treatment cycle, which treatment cycle comprises a treatment course followed by a non-treatment interval. One or more doses of the compound can be administered on one or more days.

In some embodiments, the methods comprise administering the compound in at least two treatment cycles. A non-treatment interval can be at least about 2 weeks or about 0.5 to about 12 months, such as at least about one month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. The non-treatment interval can be about 1 to about 2 years or about 1 to about 3 years, or longer. Each treatment course can be, for example, no longer than about 1 month, no longer than about 2 months, or no longer than about 3 months; or is no longer than 1, 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days.

In some embodiments, the treatment window is about one day. In some embodiments, a single treatment course occurs over no longer than 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days. During such treatment windows, the compound can be administered at least on 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days with a variable number of days on which the compound is not administered. For example, administration can be discontinued for 2, 3, 4, 5, 6, 7, 8, 9, 10, 11, 12, 13, 14, 15, 16, 17, 18, 19, 20, 21, 22, 23, 24, 25, 26, 26, 27, 28, 29, 30, or 31 days, and a discontinuation can occur at any time during the protocol. Intervals can be chosen as appropriate for the disease being treated, the compound being administered, the health status of the subject, and other relevant factors.

A daily dose of the compound can be a single administration or the dose can be divided into 2, 3, 4, or 5 separate administrations to provide the total daily dose of the compound.

A treatment cycle can be repeated as often as needed. For example, a treatment cycle can be repeated at least once, at least twice, at least three times, at least four times, at least five times, at least six times, at least seven times, at least eight times, at least nine times, at least ten times, or more often as needed. Consecutive cycles can have the same, similar, or different durations, dosages, or protocols. Treatment course or a treatment cycle can be repeated, such as when the disease or disorder recurs, or when symptoms or sequelae of the disease or disorder that were significantly diminished by one treatment course as described above have increased or are detectable, or when the symptoms or sequelae of the disease or disorder are exacerbated, a treatment course may be repeated.

A compound can be administered to a subject to reduce likelihood of occurrence or development, or to delay onset, progression, or severity of the disease, and a cycle useful for that purpose can be administered.

In some embodiments, a compound described herein is administered in a treatment window comprising 21 days. In some embodiments, the compound is administered daily for 14 days followed by 7 days off. In some embodiments, the compound is administered daily for 13 days followed by 8 days off. In some embodiments, the compound is administered daily for 12 days followed by 9 days off. In some embodiments, the compound is administered daily for 11 days followed by 10 days off. In some embodiments, the compound is administered daily for 10 days followed by 11 days off. In some embodiments, the compound is administered daily for 9 days followed by 12 days off. In some embodiments, the compound is administered daily for 8 days followed by 13 days off. In some embodiments, the compound is administered daily for 7 days followed by 14 days off. In some embodiments, the compound is administered daily for 6 days followed by 15 days off. In some embodiments, the compound is administered daily for 5 days followed by 16 days off. In some embodiments, the compound is administered daily for 4 days followed by 17 days off. In some embodiments, the compound is administered daily for 3 days followed by 18 days off. In some embodiments, the compound is administered daily for 2 days followed by 19 days off. In some embodiments, the compound is administered for 1 day followed by 20 days off.

In some embodiments, the compound is administered daily for about 21, about 14, or about 7 days in a dose of about 150 mg to about 325 mg. In some embodiments, the compound is administered daily for about 21, about 14, or about 7 days in a dose of about 150 mg to about 300 mg. In some embodiments, the compound is administered daily for about 21, about 14, or about 7 days in a dose of about 150 mg to about 275 mg. In some embodiments, the compound is administered daily for about 21, about 14, or about 7 days in a dose of about about 150 mg to about 250 mg. In some embodiments, the compound is administered daily for about 21, about 14, or about 7 days in a dose of about 150 mg to about 225 mg. In some embodiments, the compound is administered daily for about 21, about 14, or about 7 days in a dose of about 150 mg to about 200 mg. In some embodiments, the compound is administered daily for about 21, about 14, or about 7 days in a dose of about 150 mg to about 175 mg. In some embodiments, the compound is administered daily for about 21, about 14, or about 7 days in a dose of about 150 mg. In some embodiments, the compound is administered daily for about 21, about 14, or about 7 days in a dose of about 125 mg. In some embodiments, the compound is administered daily for about 21, about 14, or about 7 days in a dose of about 100 mg. In some embodiments, the compound is administered daily for about 21, about 14, or about 7 days in a dose of about 75 mg. In some embodiments, the compound is administered daily for about 21, about 14, or about 7 days in a dose of about 50 mg. In some embodiments, the compound is administered daily for about 21, about 14, or about 7 days in a dose of about 25 mg.

In some embodiments, the compound is administered in a treatment window of 28 days. In some embodiments, the compound is administered daily for 10 days, followed by 18 days off, daily for 9 days, followed by 19 days off, daily for 8 days, followed by 20 days off daily for 7 days, followed by 21 days off, daily for 6 days, followed by 22 days off, daily for 5 days, followed by 23 days off, daily for 4 days, followed by 24 days off, daily for 3 days, followed by 25 days off daily for 2 days, followed by 26 days off, or for 1 day, followed by 27 days off.

In some specific embodiments, the compound is administered daily for about 10 days in a dose of about 20 mg/m², about 19 mg/m², about 18 mg/m², about 17 mg/m², about 16 mg/m², about 15 mg/m², about 14 mg/m², about 13 mg/m², about 12 mg/m², about 11 mg/m², about 10 mg/m², about 9 mg/m², about 8 mg/m², about 7 mg/m², about 6 mg/m², about 5 mg/m², about 4 mg/m², about 3 mg/m², about 2 mg/m², about 1 mg/m², about 0.75 mg/m², about 0.5 mg/m², about 0.25 mg/m², about 0.1 mg/m², or about 0.01 mg/m². The compound can be administered for 5, 6, 7, 8, 9, 10, 11, 12, 13, or for 14 days at the doses described above.

### EXAMPLES

### Example 1: Efficacy of Compounds in an Animal Model of Osteoarthritis

C57BL/6J mice undergo surgery to cut the anterior cruciate ligament of one rear limb to induce osteoarthritis in the joint of that limb. At week 2 post-surgery, mice receive 2.5 μg of test compound to the operated knee by intra-articular injection, qd for 5 days, with a 2^{nd} treatment (2.5 μg test compound-qd for 5 days) during week 4 post-surgery. At the end of 4 weeks post-surgery, operated joints of the mice are assessed for function, monitored for markers of inflammation, and undergo histological assessment.

Two control groups of mice are included: one group comprising C57BL/6J mice that undergo a sham surgery (i.e., surgical procedures are followed except for cutting the ACL) and receive intra-articular injections of vehicle parallel to the treated group; and one group comprising C57BL/6J that undergo an ACL surgery and receive intra-articular injections of vehicle.

Function of the limbs are assessed at 4 weeks post-surgery by a weight bearing test to determine which leg the mice favor. The mice are allowed to acclimate to the chamber on at least 3 occasions prior to taking measurements. Mice are maneuvered inside the chamber to stand with 1 hind paw on each scale. The weight that is placed on each hind limb is measured over a 3-second period. At least 3 separate measurements are made for each animal at each time point. The results are expressed as the percentage of the weight placed on the operated limb versus the contralateral unoperated limb.

The function of the limbs are also assessed at 4 weeks post-surgery by hotplate analysis to show sensitivity and reaction to pain stimulus. In brief, a mouse is placed on a hotplate at 55° C. When placed on the hot surface of the plate, mice will lift their paws and lick them (paw-lick response) due to attainment of pain threshold. The latency period for the hind limb response (paw-lick response) is recorded as response time.

Histopathology of the proteoglycan layer is also analyzed.

### Example 2: Efficacy of Compounds in an Animal Model of Cardiac Stress Resistance

At 12 months of age, mice are injected three times per week with the test compound, while a control group receives vehicle. At 18 months, subsets of male and female mice are subjected to a cardiac stress test, in which mice are injected with a lethal dose of isoproterenol (680 mg/kg) and the time to cardiac arrest is recorded. The time to cardiac arrest is compared between treated and untreated animals.

### Example 3: Efficacy of Compounds in an Animal Model of Atherosclerosis

LDL^{-/-} mice from 10 weeks of age are fed a high fat diet having 42% calories from fat beginning at Week 0 until Week 12.5. The mice are then switched to a normal chow diet. Mice are treated with a test compound or a vehicle from week 12.5 over the next 100 days, with each treatment cycle comprising 5 days of test compound (25 mg/kg intraperitoneally daily) and 14 days off. At the end of the 100 day treatment period, mice are sacrificed, plasma and tissues are collected, and atherosclerosis is quantitated. Descending aortas are dissected and stained with Sudan IV to visualize the plaque lipids. The percentage of the aorta covered in plaques is measured by area, and is compared between the treated and untreated animals.

### Example 4: Efficacy of Compounds in Animal Models of Pulmonary Disease

To assess the efficacy of compounds in treating pulmonary diseases, a model of bleomycin-induced injury is used. In this model, mice develop lung fibrosis within 7-14 days after bleomycin treatment.

Bleomycin is administered to anesthetized 6-8 week-old mice by intratracheal aspiration (2.5 U/kg of bleomycin in 50 μl PBS) using a microsprayer syringe. Control mice are administered saline. The day following bleomycin treatment, test compound (25 mg/kg in PBS) or vehicle is administered. Mice are treated via intraperitoneal injection for 5 consecutive days, followed by 5 days of rest, followed by a second treatment cycle of 5 consecutive days. Untreated mice receive an equal volume of vehicle. At 7, 14, and 21 days post-bleomycin treatment, lung function is assessed by monitoring oxygen saturation using the MouseSTAT PhysioSuite pulse oximeter (Kent Scientific). Animals are anesthetized with isoflurane (1.5%) and a toe clip is applied. Mice are monitored for 30 seconds and the average peripheral capillary oxygen saturation (SpO₂) measurement over this duration is calculated.

At 21 days post-bleomycin treatment, airway hyper-reactivity (AHR) of mice is examined AHR of mice is measured by methacholine challenge while other parameters of lung function (airway mechanics, lung volume and lung compliance) are determined using a SCIREQ flexiVent ventilator. While under ketamine/xylazine anesthesia and subjected to cannulation of the trachea via a tracheostomy (19Fr blunt Luer cannula), airway resistance (elastance) and compliance of mice are assessed at baseline and in response to increasing concentrations of methacholine (0 to 50 mg/mL in PBS) delivered via nebulization (AeroNeb). Animals are maintained at 37° C., and while under muscle paralysis (pancuronium); airway function is measured by using the FlexiVent™ ventilator and lung mechanics system (SCIREQ, Montreal, Quebec, Canada).

Mice are euthanized by i.p injection of pentobarbital. Bronchoalveolar lavage (BAL) fluids and lungs is obtained and analyzed. Hydroxyproline content of lungs is measured and quantitative histopathology is performed.

In a second animal model for pulmonary diseases (e.g., COPD), mice are exposed to cigarette smoke. The effect of a test compound on the mice exposed to smoke is assessed by lung function and histopathology.

Six week-old mice are chronically exposed to cigarette smoke from a Teague TE-10 system, an automatically-controlled cigarette smoking machine that produces a combination of side-stream and mainstream cigarette smoke in a chamber, which is transported to a collecting and mixing chamber where varying amounts of air is mixed with the smoke mixture. Mice receive a total of 6 hours of cigarette smoke exposure per day, 5 days a week for 6 months. Each lighted cigarette is puffed for 2 seconds and once every minute for a total of 8 puffs, with the flow rate of 1.05 L/min, to provide a standard puff of 35 cm³. The smoke machine is adjusted to produce a mixture of side stream smoke (89%) and mainstream smoke (11%) by smoldering 2 cigarettes at one time. The smoke chamber atmosphere is monitored for total suspended particulates (80-120 mg/m³) and carbon monoxide (350 ppm). Beginning at day 7, mice are treated with test compound or vehicle (3× per week) (5 consecutive days of treatment followed by 16 days off drug, repeated until the end of the experiment), respectively. An equal number of mice received the corresponding vehicle.

After two months of cigarette smoke exposure, lung function is assessed by monitoring oxygen saturation using the MouseSTAT PhysioSuite pulse oximeter (Kent Scientific). Animals are anesthetized with isoflurane (1.5%) and the toe clip is applied. Mice are monitored for 30 seconds and the average peripheral capillary oxygen saturation (SpO₂) measurement over this duration is calculated.

At the end of the experimental period, airway hyper-reactivity (AHR) of mice to methacholine challenge using a SCIREQ flexiVent ventilator and lung mechanics system is examined as described above. After AHR measurement, mice are killed by i.p. injection of pentobarbital for in-depth analysis of lung histopathology. Briefly, lungs are inflated with 0.5% low-melting agarose at a constant pressure of 25 cm. Lungs are fixed in 10% buffered formalin and embedded in paraffin. Sections (5 μm) are stained with hematoxylin and eosin. Mean alveolar diameter, alveolar length, and mean linear intercepts are determined by computer-assisted morphometry with Image Pro Plus software (Media Cybernetics).

### Example 5: Efficacy of Compounds in Treating Chemotherapy-Induced Side Effects

Paclitaxel is administered to mice. Groups of mice (n=4) are treated three times every two days with 20 mg/kg paclitaxel or vehicle. Two days after the third dose of paclitaxel, test compound is administered daily for three days (days 1, 2, and 3) intraperitoneally at 25 mg/kg. Two days after the last dose of test compound, all groups of animals are housed in metabolic cages to monitor voluntary exercise as determined by wheel counts. Data are collected and analyzed two days later. Wheel count reduction as caused by chemotherapy is observed for restoration by the test compound.

### Example 6: Efficacy of Compounds in Improving Glucose Tolerance and Insulin Sensitivity

Groups of mice (n=9) are fed a high fat diet for four months mice or a regular chow diet. Animals are then treated with test compound (3 rounds of 25 mg/kg test compound administered daily for five consecutive days) or vehicle. A glucose bolus is given at time zero, and blood glucose is monitored at 20, 30, 60, and 120 minutes after delivering glucose to determine glucose disposal. AUC is quantitated, with a higher AUC value indicating glucose intolerance. Hemoglobin A1c level is also measured for assessing glucose tolerance. Insulin sensitivity is also determined (Insulin Tolerance Testing (ITT)). Changes in weight, body composition, and food intake are also monitored.

1. A method of selectively removing senescent cells from a cell population or tissue, comprising contacting the cell population or tissue with a compound having a chemical structure that comprises Formula (III) or Formula (IV) as shown below, a dephosphorylated form thereof, or a salt thereof: 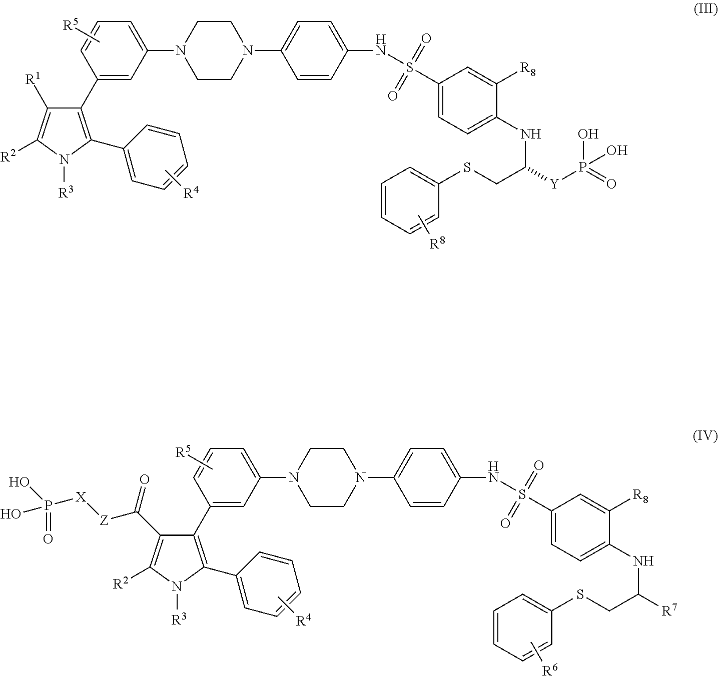
wherein:
X is substituted or unsubstituted, and is selected from alkylene, alkenylene, cycloalkylene, cycloalkenylene, and heterocycloalkylene;
Y is selected from (CH₂)ₙ—N(R^{a}) and 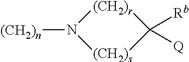
Q is selected from O, O(CH₂)₁₋₃, NR^{c}, NR^{c}(C₁₋₃alkylene), OC(═O)(C₁₋₃alkylene), C(═O)O, C(═O)O(C₁₋₃alkylene), NHC(═O)(C₁₋₃alkylene), C(═O)NH, and C(═O)NH(C₁₋₃alkylene);
Z is O or NR^{c};
R₁ and R₂, independently, are selected from H, CN, NO₂, halo, alkyl, cycloalkyl, alkenyl, cycloalkenyl, alkynyl, aryl, heteroaryl, heterocycloalkyl, OR′, SR′, NR′R″, COR′, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;
R₃ is selected from H, alkyl, cycloalkyl, alkenyl, cycloalkenyl, alkynyl, aryl, heteroaryl, heterocycloalkyl, OR′, NR′R″, OCOR′, CO₂R′, COR′, CONR′R″, CONR′SO₂R″, C₁₋₃alkyleneCH(OH)CH₂OH, SO₂R′, and SO₂NR′R″;
R′, R″, and R′″, independently, are H, alkyl, cycloalkyl, alkenyl, cycloalkenyl, alkynyl, aryl, heteroaryl, C₁₋₃alkyleneheterocycloalkyl, or heterocycloalkyl;
R′ and R″, or R″ and R′″, can be taken together with the atom to which they are bound to form a 3 to 7 membered ring;
R₄ is hydrogen, halo, C₁₋₃alkyl, CF₃, or CN;
R₅ is hydrogen, halo, C₁₋₃alkyl, substituted C₁₋₃alkyl, hydroxyalkyl, alkoxy, or substituted alkoxy;
R₆ is selected from H, CN, NO₂, halo, alkyl, cycloalkyl, alkenyl, cycloalkenyl, alkynyl, aryl, heteroaryl, heterocycloalkyl, OR′, SR′, NR′R″, CO₂R′, OCOR′, CONR′R″, CONR′SO₂R″, NR′COR″, NR′CONR″R′″, NR′C═SNR″R′″, NR′SO₂R″, SO₂R′, and SO₂NR′R″;
R₇, substituted or unsubstituted, is selected from hydrogen, alkyl, alkenyl, (CH₂)₀₋₃cycloalkyl, (CH₂)₀₋₃cycloalkenyl, (CH₂)₀₋₃heterocycloalkyl, (CH₂)₀₋₃aryl, and (CH₂)₀₋₃heteroaryl;
R₈ is selected from hydrogen, halo, NO₂, CN, SO₂CF₃, and CF₃;
Rₐ is selected from hydrogen, alkyl, heteroalkyl, alkenyl, hydroxyalkyl, alkoxy, substituted alkoxy, cycloalkyl, cycloalkenyl, and heterocycloalkyl;
R_{b} is hydrogen or alkyl;
R_{c} is selected from hydrogen, alkyl, substituted alkyl, hydroxyalkyl, alkoxy, and substituted alkoxy; and
n, r, and s, independently, are 1, 2, 3, 4, 5, or 6.

2. The method of claim 1, wherein the compound has a chemical structure that comprises any one of the structures shown below, a dephosphorylated form thereof, or a salt thereof:

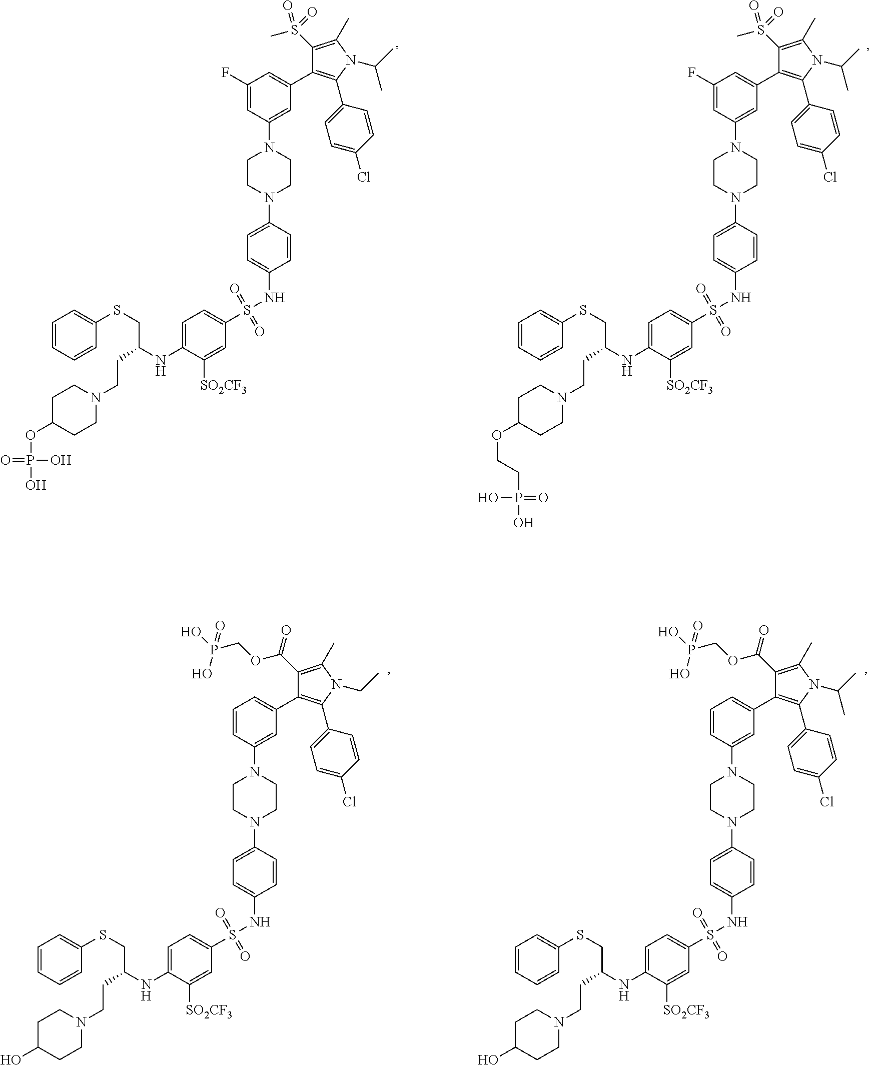

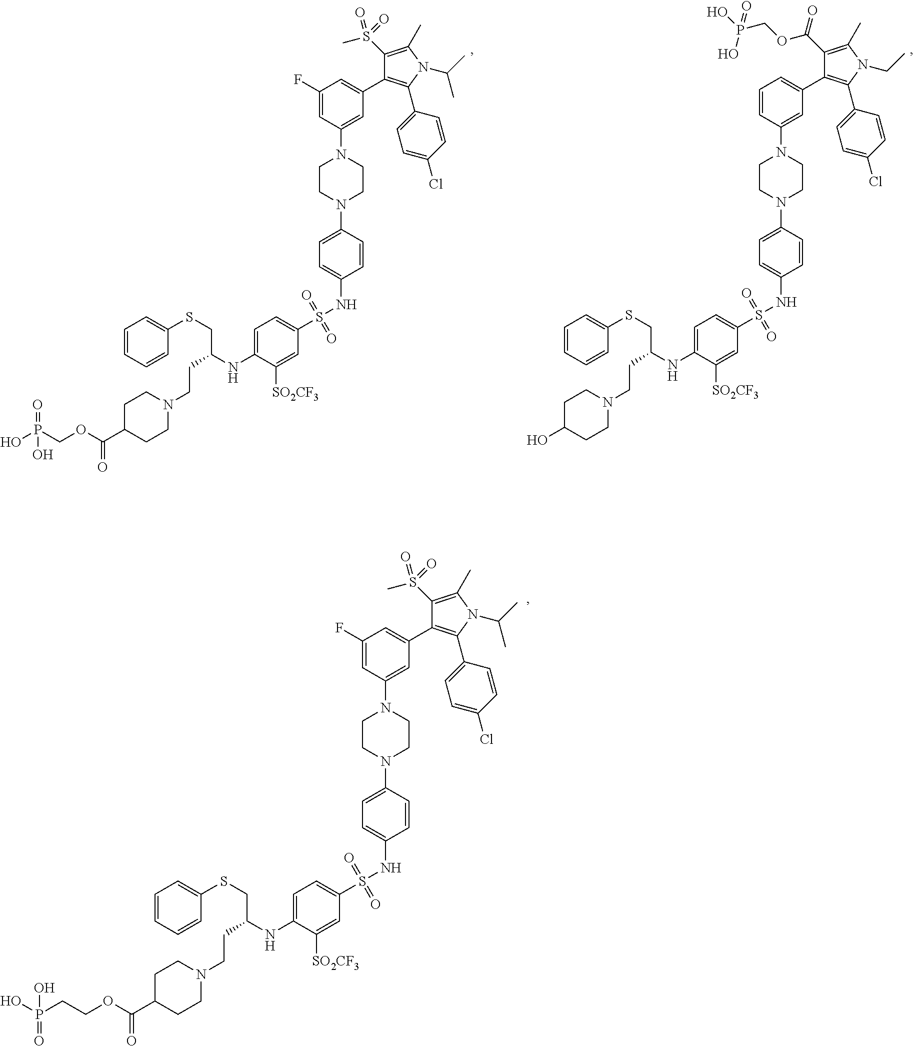

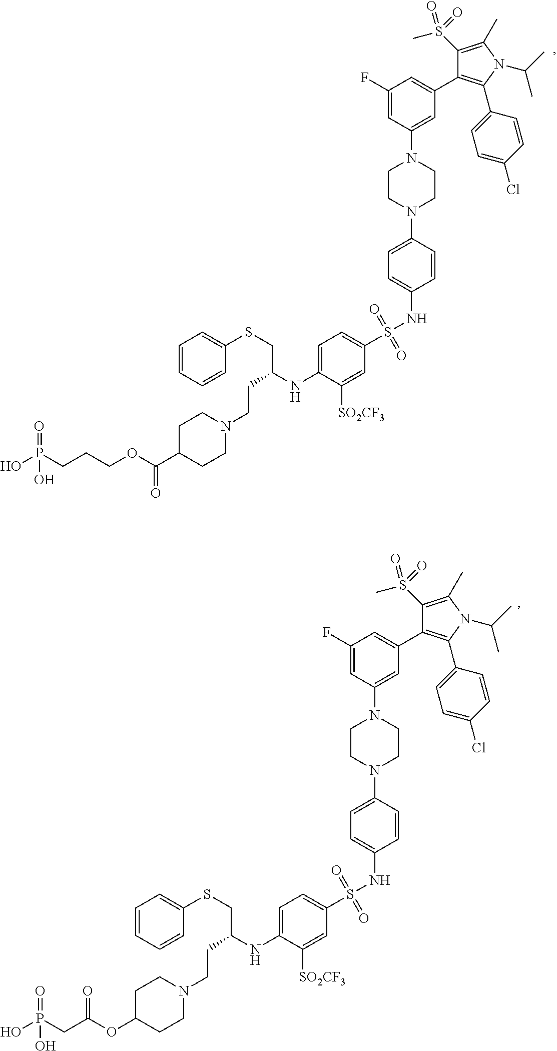

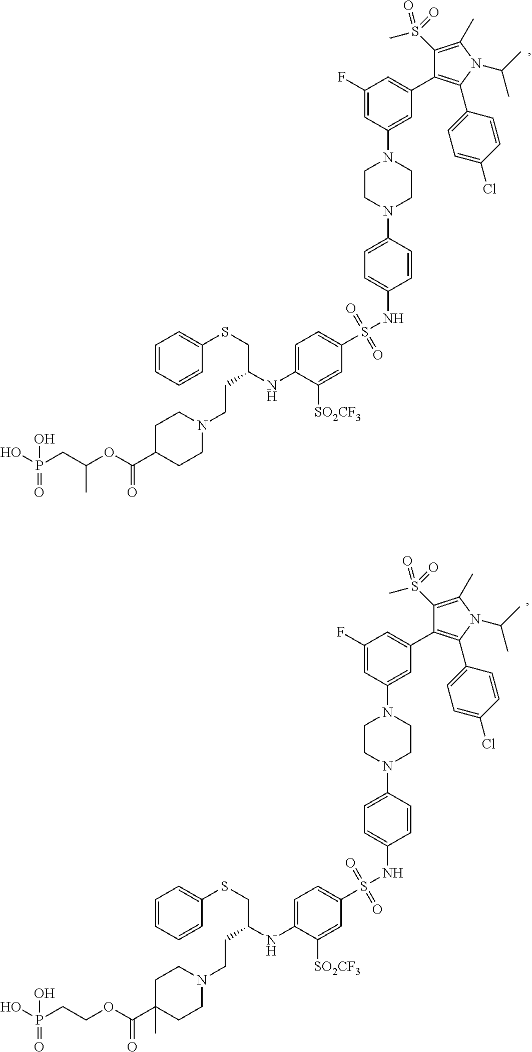

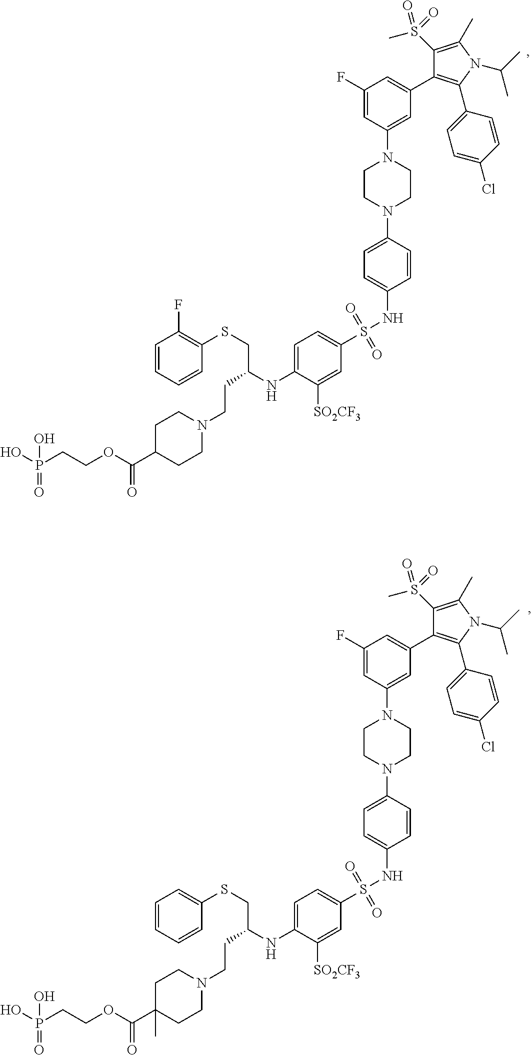

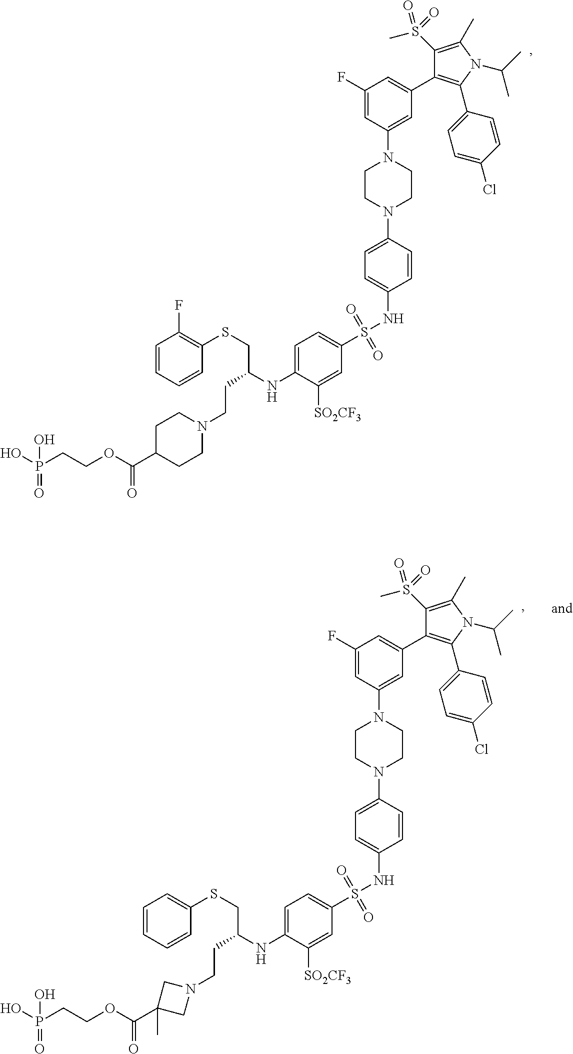

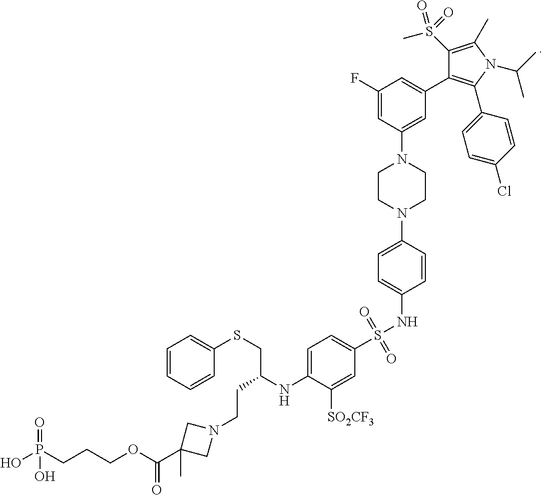

3. The method of claim 2, wherein the compound is a phosphorylated form of any one of the structures shown in claim 2, or a salt thereof.

4. The method of claim 2, wherein the compound is a dephosphorylated form of any one of the structures shown in claim 2 or a salt thereof.

5. The method of claim 1, wherein the cell population or tissue is contacted with the compound in vitro.

6. The method of claim 1, wherein the cell population or tissue is in an osteoarthritic joint.

7. The method of claim 1, wherein the cell population or tissue is in an atherosclerotic plaque.

8. The method of claim 1, wherein the cell population or tissue is in pulmonary tissue.

9. The method of claim 8, wherein the cell population or tissue is in a tissue that has been chronically exposed to cigarette smoke.

10. The method of claim 1, wherein the cell population or tissue consists of a single senescent cell.

11. The method of claim 1, wherein the cell population or tissue contains senescent fibroblasts.

12. The method of claim 1, wherein the cell population or tissue contains senescent preadiposites.

13. The method of claim 1, wherein the compound is administered in an amount less than an amount that would be effective for removing cancer cells from the cell population or tissue.

14. The method of claim 1, wherein the compound is administered in a plurality of treatment cycles, wherein each treatment cycle independently comprises a treatment course of from 1 day to 3 months followed by a non-treatment interval of at least 2 weeks

15. The method of claim 1, wherein the compound is administered as a single dose followed by a non-treatment interval of at least 2 weeks.

16. A method for treating a senescence-associated condition, comprising administering to a subject in need thereof a compound having a chemical structure that comprises Formula (III) or Formula (IV) as shown above, a dephosphorylated form thereof, or a salt thereof.

17. The method of claim 16, wherein the senescence-associated condition is osteoarthritis, atherosclerosis, or a pulmonary condition.

18. A pharmaceutical product that includes a pharmaceutical composition that contains a compound having a chemical structure that comprises Formula (III) or Formula (IV) as shown above, a dephosphorylated form thereof, or a salt thereof, in combination with a pharmaceutically compatible excipient; wherein the pharmaceutical product is packaged with information for administration or use of the composition for the treatment of osteoarthritis, atherosclerosis, or a pulmonary condition.
